(19) United States
(12) Patent Application Publication
(10) Pub. No.: US 2026/0216238 A1
(43) Pub. Date: Jul. 30, 2026

(54) TETRATHIOMOLYBDIC ACID FOR USE IN TREATING COPPER METABOLISM-ASSOCIATED DISEASES OR DISORDERS (WILSON DISEASE)

(71) Applicant: Alexion Pharmaceuticals, Inc., Boston, MA (US)

(72) Inventors: Eugene Scott Swenson, Boston, MA (US); Jennifer Lee Pocoski, Boston, MA (US); Megan Teynor, Boston, MA (US); Liliana-Georgiana Wegmann, Boston, MA (US); Brian Meltzer, Boston, MA (US); Barbara Davies, Boston, MA (US)

(21) Appl. No.: 19/152,046

(22) PCT Filed: Feb. 16, 2023
(86) PCT No.: PCT/US23/13230

§ 371 Date: Jul. 30, 2025

Related U.S. Application Data
(60) Provisional application No. 63/482,160, filed on Jan. 30, 2023.

(51) Int. Cl. A61K 33/24; A61P 3/00

(52) U.S. Cl. CPC A61K 33/24; A61P 3/00

(57) ABSTRACT

This disclosure relates to methods of treating a copper metabolism-associated disease or disorder, such as Wilson disease (WD).

### BACKGROUND OF THE DISCLOSURE

### Field of the Disclosure

This disclosure relates to methods of treating a copper metabolism-associated disease or disorder, such as Wilson disease (WD).

### Description of Related Art

Wilson disease (WD) is an autosomal recessive disorder of impaired copper transport. Mutations in the ATP7B gene result in deficient production of the copper-transporter ATPase2, leading to impaired incorporation of copper into ceruloplasmin (Cp), impaired biliary excretion of copper, increased exchangeable copper, and copper accumulation in liver, brain, and other tissues, with resulting organ damage and dysfunction. Ceruloplasmin is a serum ferroxidase, and in healthy humans, it contains greater than 95% of the copper found in plasma.

The prevalence of genetic markers associated with WD is approximately one per 30,000 population worldwide. Among people with an identified mutation, disease manifestation will be present in approximately 50%. The majority of patients are diagnosed before 30 years of age. A recent nationwide, population-based epidemiological study based in France found the diagnosed prevalence of WD to be 1.5 per 100,000 population.

Typical clinical presentation of WD is in adolescence to early adulthood. Genetic screening and genotype-phenotype correlation is complicated by a multitude (>500) of associated ATP7B mutations; most individuals with WD are compound heterozygotes. Initial signs and symptoms of WD are predominantly hepatic (~40%), neurologic (~40%), or psychiatric (~20%), but patients often develop combined hepatic and neuropsychiatric disease. Untreated or inadequately treated patients have progressive morbidity, and mortality is usually secondary to hepatic cirrhosis. Liver transplantation is the only effective therapy for WD-associated acute liver failure; other causes of death associated with WD include hepatic malignancy and neurologic deterioration with severe inanition.

Disease control in patients with neurological symptoms at WD diagnosis is an area of particular concern. More than one-third of patients presenting with neurological symptoms show no improvement after 4 years of treatment with chelators. This failure to respond to chelation therapy with neurological presentation may reflect irreversible damage to the nervous system. Also, in a recent study, approximately 50% of patients had residual neurological symptoms despite years of therapy on a copper-modulating agent. Worsening of neurological symptoms on initiation of treatment has been reported in approximately 25% of patients initiated on penicillamine and trientine, and up to 50% of those patients never recover. The mechanism behind this worsening is believed to be a mobilization of copper from the liver leading to elevations in brain copper associated with neurological progression. This theory is supported by non-clinical data. Copper accumulation in the central nervous system is an evolutionary and long-term process. Excessive copper in the body as a whole (not just from the liver) may gradually accumulate in the central nervous system over time.

Currently available drugs have high rates of treatment discontinuation due to adverse events (AEs) and treatment failure. They also need to be dosed 2 to 5 times per day and must be taken in the fasted state. Their AE profiles and complicated dosing regimens lead to poor treatment compliance and high rates of treatment failure, a major concern in a disease that requires life-long treatment such as WD. Further, although standard of care treatments for WD are considered to have acceptable safety profiles, continuous monitoring is required to identify potential adverse effects in a timely fashion, to confirm treatment efficacy and to ensure adherence to medication.

Therefore, there remains a need for improvements over the standard of care treatments of the copper metabolism-associated diseases or disorders.

### SUMMARY OF THE DISCLOSURE

The disclosure generally provides methods useful for treating a copper metabolism-associated disease or disorder, such as Wilson disease, in a subject.

In one embodiment, the disclosure provides a method for treating a copper metabolism disease or disorder in a subject, the method including administering to the subject a therapeutically effective amount of tetrathiomolybdic acid (“TTM”) or a pharmaceutically acceptable salt thereof, wherein the subject is at least 12 years old; and wherein the subject had an incomplete response and/or is intolerant to a standard of care treatment. For purposes of this disclosure, a subject who had an incomplete response may also be called an “incomplete responder” or an “inadequate responder.”

In some embodiments of the methods, uses, or compositions described herein, the copper metabolism disease or disorder is Wilson disease.

In another embodiment, the disclosure provides tetrathiomolybdic acid or a pharmaceutically acceptable salt thereof for treating a copper metabolism associated disease or disorder in a subject, wherein a therapeutically effective amount of tetrathiomolybdic acid or a pharmaceutically acceptable salt thereof is administered to the subject; wherein the subject is at least 12 years old; and wherein the subject had an incomplete response and/or is intolerant to a standard of care treatment.

In another embodiment, the disclosure provides a use of tetrathiomolybdic acid or a pharmaceutically acceptable salt thereof for the manufacture of a medicament for treating a copper metabolism associated disease or disorder in a subject, wherein a therapeutically effective amount of tetrathiomolybdic acid or a pharmaceutically acceptable salt thereof is administered to the subject; wherein the subject is at least 12 years old; and wherein the subject had an incomplete response and/or is intolerant to a standard of care treatment.

These and other features and advantages of the claimed invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

The accompanying drawings are included to provide a further understanding of the compositions and methods of the disclosure, and are incorporated in and constitute a part of this specification. The drawings illustrate one or more embodiment(s) of the disclosure and, together with the description, serve to explain the principles and operation of the disclosure.

FIG. 1A is a schematic representation of the study provided in the Example 1. Abbreviation: SoC=standard of care.

FIG. 1B is a schematic representation of the enrolment and study design of the study provided in the Example 1.

FIG. 2 shows plasma total copper, directly measured non-ceruloplasmin-bound copper (dNCC) values and 24 h-urine copper over time, by cohort in TTM treated participants, mean and 95% Cl (Full Analysis Set). Red dash line is the lower limit of the normal reference range for plasma total copper: 11.3 μmol/L.

FIG. 3 shows plasma total copper, directly measured NCC and 24 h-urine copper overtime, by cohort in SoC treated participants, mean and 95% Cl (Full Analysis Set). Red dash line is the lower limit of the normal reference range for plasma total copper: 11.3 μmol/L.

FIG. 4 shows plasma total copper, directly measured NCC and 24 h-urine copper over time, by cohort in zinc monotherapy treated participants, mean and 95% Cl (Full Analysis Set). Red dash line is the lower limit of the normal reference range for plasma total copper: 11.3 μmol/L. No Cohort 2 participants were treated with zinc monotherapy.

FIG. 5 shows plasma total copper, directly measured NCC and 24 h-urine copper overtime, by cohort in penicillamine (+/−zinc) treated participants, mean and 95% Cl (Full Analysis Set). Red dash line is the lower limit of the normal reference range for plasma total copper: 11.3 μmol/L.

FIG. 6 shows plasma total copper, directly measured NCC and 24 h-urine copper overtime, by cohort in trientine (+/−zinc) treated participants, mean and 95% Cl (Full Analysis Set). Red dash line is the lower limit of the normal reference range for plasma total copper: 11.3 μmol/L.

FIG. 7 shows boxplots of plasma CpC/Cp ratio by treatment (TTM vs SoC) in Study 301.

FIG. 8 shows box plots of the calculated daily mean AUC_{(0-48 weeks)} values for plasma total and ultrafiltrate molybdenum by age group (adult vs adolescent) (PK Analysis Set).

FIG. 9 shows box plots of calculated daily mean AUEC_{(0-48 weeks)} values for plasma total copper (PTC), dNCC, and LBC by age group (adult vs adolescent) (PD and Biomarker Analysis Set).

FIG. 10 shows increase from baseline in directly measured NCC (μmol/L) in Plasma for each of 3 patients in Study 204.

FIG. 11 shows increase from baseline in daily fecal copper excretion (mg) for each of 3 patients in Study 204.

FIG. 12 shows decrease from baseline in average daily net copper balance (mg) for each of 3 patients in Study 204.

FIG. 13 shows improvement in UWDRS Part II Score (range 0-40) least square means and standard errors over 5 years of TTM from baseline (Pooling Full Analysis Set, Studies 301 and 201). X-axis legend: Red=Number of participants; Black=Weeks from TTM treatment initiation.

FIG. 14 shows improvement in UWDRS Part III Score (range 0-175) least square means and standard errors over 5 years of TTM from baseline (Pooling Full Analysis Set, Studies 301 and 201). Note: X-axis legend: Red=Number of participants; Black=Weeks from TTM treatment initiation.

FIG. 15 shows improvement in UWDRS Part III functional subscale score (range 0-10) least square means and standard errors over 5 years of TTM from baseline (Pooling Full Analysis Set, Studies 301 and 201). Note: X-axis legend: Red=Number of participants; Black=Weeks from TTM treatment initiation.

FIG. 16 shows ALT overtime mean with 95% Cl (Primary Evaluation Period-Safety Set).

FIG. 17 shows GGT over time mean with 95% Cl (Primary Evaluation Period-Safety Set).

FIG. 18 shows total cholesterol overtime: mean with 95% Cl (Primary Evaluation Period—Safety Set). Note: Upper limit of normal: 5.17 mmol/L=200 mg/dL.

FIG. 19 shows triglyceride over time: mean with 95% Cl (Primary Evaluation Period—Safety Set). Note: Upper limit of normal: 1.69 mmol/L=150 mg/dL.

FIG. 20 shows improvements in UWDRS Part II scores for most groups at week 24. ^{a}Symptomatic patients were those with UWDRS part II score>0 at baseline; data for these subgroups are from a post-hoc analysis.

FIG. 21 shows improvements in UWDRS Part III scores for most groups at week 24. ^{a}Symptomatic patients were those with UWDRS part III score>0 at baseline; data for these subgroups are from a post-hoc analysis.

FIG. 22 shows improvements in CGI-I scores with TTM at week 48 versus SoC.

FIG. 23 shows 24-hour urinary copper concentration (μmol/day, mean (SD)). P/T, penicillamine/trientine; Zn, zinc.

FIG. 24 shows the net promoter score for UWDRS Part II total scores for the incomplete or intolerant response group.

FIG. 25 shows the heterogeneity of symptoms at baseline for the incomplete or intolerant response group.

FIG. 26 shows the individual symptom response for the incomplete or intolerant response group when treated with TTM and standard of care.

FIG. 27 shows the improvement in UWDRS Part III Score (range 0-175) least square means and standard errors over 5 years of TTM treatment from baseline.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

In view of the present disclosure, the methods, uses, and compositions (e.g., comprising tetrathiomolybdic acid or a pharmaceutically acceptable salt thereof) described herein can be configured by the person of ordinary skill in the art to meet the desired need. The present disclosure provides improvements in treating copper metabolism-associated diseases or disorders.

Approximately half of newly-diagnosed patients with WD are younger than 18 years old. Standard treatments for WD are approved for use in children or adolescents, but significant unmet needs still exist with respect to efficacy, safety, and simplicity of dosing regimens. All currently available WD treatments are associated with adverse effects (such as neurological worsening) in a subset of patients, which can require adjustment, substitution, or even discontinuation of treatment. These adverse effects also reduce the patient's compliance with treatment, which by itself can lead to clinical deterioration and even death. All require multiple daily doses to achieve adequate copper control. The burden of multiple daily doses for standard treatments may negatively impact medical adherence and clinical outcomes, particularly among patients who discontinue treatment entirely.

As described above, there is a need in the art to improve the standard of care treatments for copper metabolism-associated diseases or disorders, especially WD. Tetrathiomolybdic acid (“TTM”) or pharmaceutically acceptable salts thereof are alternatives to the standard of care treatment. For example, bis-choline tetrathiomolybdate (“BC-TTM”) (also known as ALXN1840, tiomolibdate choline, and tiomolibdic acid; formerly known as WTX101) is an investigational, oral, first-in-class copper-protein-binding molecule being developed for the treatment of WD. BC-TTM has the following structure:

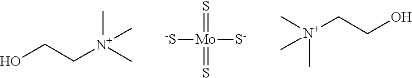

and has been described in detail in International Publication No. WO 2019/110619 (incorporated by reference herein in its entirety). It is hoped that improved long-term compliance with TTM treatment through improved tolerability and the convenience of a simplified once daily (QD) dosing regimen compared with current therapeutic options could be achieved. Furthermore, once-daily dosing and the small tablet diameter of the 7.8 mg dose of TTM (5 mm) may increase therapeutic adherence.

In one embodiment, the present disclosure as described herein provides a method for treating a copper metabolism-associated disease or disorder in a subject, the method including administering to the subject a therapeutically effective amount of tetrathiomolybdic acid or a pharmaceutically acceptable salt thereof; wherein the subject is at least 12 years old; and wherein the subject had an incomplete response and/or is intolerant to a standard of care treatment. For example, in at least one embodiment, the subject who had an incomplete response may also be called an “incomplete responder” or an “inadequate responder.”

Another aspect of the present disclosure provides tetrathiomolybdic acid or a pharmaceutically acceptable salt thereof for treating a copper metabolism associated disease or disorder in a subject as described herein. The tetrathiomolybdic acid or a pharmaceutically acceptable salt thereof for treating a copper metabolism associated disease or disorder in a subject includes administering to the subject a therapeutically effective amount of tetrathiomolybdic acid or a pharmaceutically acceptable salt thereof as described herein; wherein the subject is at least 12 years old; and wherein the subject had an incomplete response and/or is intolerant to a standard of care treatment as described herein. For example, in at least one embodiment, the subject who had an incomplete response may also be called an “incomplete responder” or an “inadequate responder.”

Another aspect of the present disclosure provides a use of tetrathiomolybdic acid or a pharmaceutically acceptable salt thereof for the manufacture of a medicament for treating a copper metabolism associated disease or disorder in a subject. The use includes administering to the subject a therapeutically effective amount of tetrathiomolybdic acid or a pharmaceutically acceptable salt thereof as described herein; wherein the subject is at least 12 years old; and wherein the subject had an incomplete response and/or is intolerant to a standard of care treatment as described herein. For example, in at least one embodiment, the subject who had an incomplete response may also be called an “incomplete responder” or an “inadequate responder.”

In certain embodiments of the methods, uses, or compositions (e.g., comprising tetrathiomolybdic acid or a pharmaceutically acceptable salt thereof) of the disclosure as described herein, the copper metabolism-associated disease or disorder is Wilson disease.

In certain embodiments of the methods, uses, or compositions (e.g., comprising tetrathiomolybdic acid or a pharmaceutically acceptable salt thereof) of the disclosure, the copper metabolism-associated disease or disorder is copper toxicity (e.g., from high exposure to copper sulfate fungicides, ingesting drinking water high in copper, overuse of copper supplements, etc.). In certain embodiments of the methods, uses, or compositions (e.g., comprising tetrathiomolybdic acid or a pharmaceutically acceptable salt thereof) of the disclosure, the copper metabolism-associated disease or disorder is copper deficiency, Menkes disease, or aceruloplasminemia. In certain embodiments of the methods, uses, or compositions (e.g., comprising tetrathiomolybdic acid or a pharmaceutically acceptable salt thereof) of the disclosure, the copper metabolism-associated disease or disorder is at least one selected from academic underachievement, acne, attention-deficit/hyperactivity disorder, amyotrophic lateral sclerosis (ALS), atherosclerosis, autism, Alzheimer's disease, Candida overgrowth, chronic fatigue, cirrhosis, depression, elevated adrenaline activity, elevated cuproproteins, elevated norepinephrine activity, emotional meltdowns, fibromyalgia, frequent anger, geriatric-related impaired copper excretion, high anxiety, hair loss, hepatic disease, hyperactivity, hypothyroidism, intolerance to estrogen, intolerance to birth control pills, Kayser-Fleischer rings, learning disabilities, low dopamine activity, multiple sclerosis, neurological problems, oxidative stress, Parkinson's disease, poor concentration, poor focus, poor immune function, ringing in ears, allergies, sensitivity to food dyes, sensitivity to shellfish, skin metal intolerance, skin sensitivity, sleep problems, and white spots on fingernails.

As used herein, the terms “treatment” and “treating” mean (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof, or inhibiting the progression of disease; or (ii) eliciting the referenced biological effect.

As used herein, the terms “individual,” “patient,” or “subject” are used interchangeably, and refer to any animal, including mammals, and, in at least one embodiment of the methods, uses, or compositions (e.g., comprising tetrathiomolybdic acid or a pharmaceutically acceptable salt thereof) of the disclosure, humans. In certain embodiments of the methods, uses, or compositions (e.g., comprising tetrathiomolybdic acid or a pharmaceutically acceptable salt thereof) of the disclosure, the subject is a healthy subject. In certain embodiments of the methods, uses, or compositions (e.g., comprising tetrathiomolybdic acid or a pharmaceutically acceptable salt thereof) of the disclosure, the subject suffers from WD. In certain embodiments of the methods, uses, or compositions (e.g., comprising tetrathiomolybdic acid or a pharmaceutically acceptable salt thereof) of the disclosure as described herein, the subject has cirrhosis. In certain other embodiments of the methods, uses, or compositions (e.g., comprising tetrathiomolybdic acid or a pharmaceutically acceptable salt thereof) of the disclosure, the subject does not have cirrhosis. In certain embodiments of the methods, uses, or compositions (e.g., comprising tetrathiomolybdic acid or a pharmaceutically acceptable salt thereof) of the disclosure, the subject is at least 18 years old.

In some embodiments of the methods, uses, or compositions (e.g., comprising tetrathiomolybdic acid or a pharmaceutically acceptable salt thereof) of the disclosure, the subject previously received the standard of care treatment for the copper metabolism-associated disease or disorder for at least 28 days.

In certain embodiments of the methods, uses, or compositions (e.g., comprising tetrathiomolybdic acid or a pharmaceutically acceptable salt thereof) of the disclosure, the subject has received standard of care treatment for WD for at least 4 weeks. In certain embodiments of the methods, uses, or compositions (e.g., comprising tetrathiomolybdic acid or a pharmaceutically acceptable salt thereof) of the disclosure, the standard of care treatment was at least 6 weeks, or at least 12 weeks, or at least 24 weeks, or at least 36 weeks, or at least 48 weeks, or at least 52 weeks, or at least 72 weeks. In certain embodiments of the methods, uses, or compositions (e.g., comprising tetrathiomolybdic acid or a pharmaceutically acceptable salt thereof) of the disclosure, the subject previously received a standard of care treatment for the copper metabolism-associated disease or disorder for at least 28 months, for at least 36 months, for at least 42 months, or for at least 48 months. For example, in some embodiments of the methods, uses, or compositions (e.g., comprising tetrathiomolybdic acid or a pharmaceutically acceptable salt thereof) of the disclosure, the subject previously received a standard of care treatment for the copper metabolism-associated disease or disorder for at least 72 months, for at least 96 months, for at least 120 months, or for at least 142 months.

The standard of care treatment need not be continuous. For example, in certain embodiments of the methods, uses, or compositions (e.g., comprising tetrathiomolybdic acid or a pharmaceutically acceptable salt thereof) of the disclosure, the subject may receive the treatment on-and-off totaling at least 4 weeks (e.g., at least 6, or at least 12, or at least 24, or at least 36, or at least 48, or at least 52 weeks, or at least 72 weeks, or at least 28 months, or at least 28 months, at least 36 months, at least 42 months, or at least 48 months, at least 72 months, at least 96 months, at least 120 months, or at least 142 months) of treatment. In certain embodiments of the methods, uses, or compositions (e.g., comprising tetrathiomolybdic acid or a pharmaceutically acceptable salt thereof) of the disclosure, however, the standard of care treatment is continuous.

In the methods, uses, or compositions (e.g., comprising tetrathiomolybdic acid or a pharmaceutically acceptable salt thereof) of the disclosure as described herein, the subject completed the standard of care treatment at least 2 weeks prior to administering tetrathiomolybdic acid or a pharmaceutically acceptable salt thereof. In certain embodiments of the methods, uses, or compositions (e.g., comprising tetrathiomolybdic acid or a pharmaceutically acceptable salt thereof) of the disclosure, the subject completed the standard of care treatment at least 3 weeks, at least 4 weeks, or at least 6 weeks prior to administering tetrathiomolybdic acid or a pharmaceutically acceptable salt thereof.

In some embodiments of the methods, uses, or compositions (e.g., comprising tetrathiomolybdic acid or a pharmaceutically acceptable salt thereof) of the disclosure as described herein, the subject has symptoms associated with the copper metabolism-associated disease or disorder after receiving the standard of care treatment for at least 26 weeks for at least 52 weeks, for at least 72 weeks, for at least 84 weeks, or for at least 96 weeks.

As described above, in various embodiments of the methods, uses, or compositions (e.g., comprising tetrathiomolybdic acid or a pharmaceutically acceptable salt thereof) of the disclosure as described herein, the subject had an incomplete response and/or is intolerant to a standard of care treatment. In certain embodiments of the methods, uses, or compositions (e.g., comprising tetrathiomolybdic acid or a pharmaceutically acceptable salt thereof) of the disclosure, the subject is an incomplete responder to the standard of care treatment. As used herein, an incomplete responder (also known as an “inadequate responder”) has an incomplete or inadequate response to the standard of care treatment. For example, in some embodiments of the methods, uses, or compositions (e.g., comprising tetrathiomolybdic acid or a pharmaceutically acceptable salt thereof) of the disclosure, the incomplete or inadequate responder has symptoms associated with the copper metabolism-associated disease or disorder after receiving the standard of care treatment for at least 26 weeks, for at least 52 weeks, for at least 72 weeks, for at least 84 weeks, or for at least 96 weeks.

In some embodiments of the methods, uses, or compositions (e.g., comprising tetrathiomolybdic acid or a pharmaceutically acceptable salt thereof) of the disclosure, the incomplete or inadequate responder has a Unified Wilson Disease Rating Scale (UWDRS) Part II total score of greater than 0 or a Unified Wilson Disease Rating Scale (UWDRS) Part III total score of greater than 0 prior to the administering the therapeutically effective amount of tetrathiomolybdic acid or a pharmaceutically acceptable salt thereof. For example, in certain embodiments of the methods, uses, or compositions (e.g., comprising tetrathiomolybdic acid or a pharmaceutically acceptable salt thereof) described herein, the subject who had an incomplete or inadequate response to a standard of care treatment has a Unified Wilson Disease Rating Scale (UWDRS) Part II total score of greater than 0 and a Unified Wilson Disease Rating Scale (UWDRS) Part III total score of greater than 0 prior to the administering the therapeutically effective amount of tetrathiomolybdic acid or a pharmaceutically acceptable salt thereof.

In some embodiments of the methods, uses, or compositions (e.g., comprising tetrathiomolybdic acid or a pharmaceutically acceptable salt thereof) described herein, the subject who had an incomplete or inadequate response to a standard of care treatment, prior to the administering a therapeutically effective amount of tetrathiomolybdic acid or a pharmaceutically acceptable salt thereof, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

In some embodiments of the methods, uses, or compositions (e.g., comprising tetrathiomolybdic acid or a pharmaceutically acceptable salt thereof) described herein, the subject who had an incomplete or inadequate response to a standard of care treatment, prior to the administering a therapeutically effective amount of tetrathiomolybdic acid or a pharmaceutically acceptable salt thereof, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

In some embodiments of the methods, uses, or compositions (e.g., comprising tetrathiomolybdic acid or a pharmaceutically acceptable salt thereof) described herein, the subject who had an incomplete or inadequate response to a standard of care treatment, prior to the administering a therapeutically effective amount of tetrathiomolybdic acid or a pharmaceutically acceptable salt thereof, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

In some embodiments of the methods, uses, or compositions (e.g., comprising tetrathiomolybdic acid or a pharmaceutically acceptable salt thereof) described herein, the subject who had an incomplete or inadequate response to a standard of care treatment, prior to the administering a therapeutically effective amount of tetrathiomolybdic acid or a pharmaceutically acceptable salt thereof, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

In some embodiments of the methods, uses, or compositions (e.g., comprising tetrathiomolybdic acid or a pharmaceutically acceptable salt thereof) described herein, the subject is intolerant to the standard of care treatment. For example, in some embodiments of the methods, uses, or compositions (e.g., comprising tetrathiomolybdic acid or a pharmaceutically acceptable salt thereof) described herein, the subject has previously been determined as intolerant to standard of care treatment by a medical professional or has had an adverse event after the receiving the standard of care treatment and prior to administering the therapeutically effective amount of tetrathiomolybdic acid or a pharmaceutically acceptable salt thereof. In some embodiments of the methods, uses, or compositions (e.g., comprising tetrathiomolybdic acid or a pharmaceutically acceptable salt thereof) of the disclosure, the subject has previously been determined as intolerant to standard of care treatment by a medical professional and has had an adverse event after receiving the standard of care treatment and prior to the administering the therapeutically effective amount of tetrathiomolybdic acid or a pharmaceutically acceptable salt thereof. In some embodiments of the methods, uses, or compositions (e.g., comprising tetrathiomolybdic acid or a pharmaceutically acceptable salt thereof) described herein, the subject has symptoms associated with the copper metabolism-associated disease or disorder after receiving the standard of care treatment for at least 26 weeks, for at least 52 weeks, for at least 72 weeks, for at least 84 weeks, or for at least 96 weeks.

The methods, uses, or compositions (e.g., comprising tetrathiomolybdic acid or a pharmaceutically acceptable salt thereof) of the disclosure are also useful as a second line treatment and/or a first line maintenance treatment of WD. Thus, in certain embodiments of the methods, uses, or compositions (e.g., comprising tetrathiomolybdic acid or a pharmaceutically acceptable salt thereof) of the disclosure, the subject has previously received a standard of care (SoC) treatment for WD. For example, in certain embodiments of the methods, uses, or compositions (e.g., comprising tetrathiomolybdic acid or a pharmaceutically acceptable salt thereof) of the disclosure, the subject has previously received trientine (also known as triethylenetatramine; N′-[2-(2-aminoethylamino)ethyl]ethane-1,2-diamine). Trientine may be sold in the form of a tetrahydrochloride salt under the names CUPRIOR® (GMP-Orphan United Kingdom Ltd) or CUVRIOR™ (Orphalan), in the form of a hydrochloride salt under the name SYPRINE® (Aton Pharma, Inc., Bausch Health), or in the form of a dihydrochloride salt under the name CUFENCE (Univar, Inc.). In certain other embodiments of the methods, uses, or compositions (e.g., comprising tetrathiomolybdic acid or a pharmaceutically acceptable salt thereof) of the disclosure, the subject has previously received trientine and zinc. In certain embodiments of the methods, uses, or compositions (e.g., comprising tetrathiomolybdic acid or a pharmaceutically acceptable salt thereof) of the disclosure, the subject has previously received D-penicillamine (also known as penicillamine; (2S)-2-amino-3-methyl-3-sulfanylbutanoic acid). D-penicillamine may be sold under the name CUPRIMINE® (Bausch Health) or DEPEN® (Meda Pharmaceuticals). In certain other embodiments of the methods, uses, or compositions (e.g., comprising tetrathiomolybdic acid or a pharmaceutically acceptable salt thereof) of the disclosure, the subject has previously received D-penicillamine and zinc. In certain embodiments of the methods, uses, or compositions (e.g., comprising tetrathiomolybdic acid or a pharmaceutically acceptable salt thereof) of the disclosure, the subject has previously received zinc. In certain embodiments of the methods, uses, or compositions (e.g., comprising tetrathiomolybdic acid or a pharmaceutically acceptable salt thereof) of the disclosure, the subject has previously received trientine, D-penicillamine, and/or zinc. In certain other embodiments of the methods, uses, or compositions (e.g., comprising tetrathiomolybdic acid or a pharmaceutically acceptable salt thereof) of the disclosure, the subject has previously received trientine and/or D-penicillamine. For example, in some embodiments of the methods, uses, or compositions (e.g., comprising tetrathiomolybdic acid or a pharmaceutically acceptable salt thereof) of the disclosure, the standard of care treatment comprises trientine, D-penicillamine, and/or zinc. In certain other embodiments of the methods, uses, or compositions (e.g., comprising tetrathiomolybdic acid or a pharmaceutically acceptable salt thereof) of the disclosure, the standard of care treatment comprises trientine and/or D-penicillamine.

As provided above, tetrathiomolybdic acid or a pharmaceutically acceptable salt thereof is administered in the methods, uses, and compositions of the disclosure. In some embodiments of the methods, uses, or compositions (e.g., comprising tetrathiomolybdic acid or a pharmaceutically acceptable salt thereof) of the disclosure as described herein, the administering of the therapeutically effective amount of tetrathiomolybdic acid or a pharmaceutically acceptable salt thereof is for at least 24 weeks, for at least 36 weeks, or for at least 48 weeks. In certain embodiments of the methods, uses, or compositions (e.g., comprising tetrathiomolybdic acid or a pharmaceutically acceptable salt thereof) of the disclosure, the therapeutically effective amount of tetrathiomolybdic acid or a pharmaceutically acceptable salt thereof is administered to the subject in a fasted state.

A therapeutically effective amount of tetrathiomolybdic acid has been previously established. For example, in certain embodiments of the methods, uses, or compositions (e.g., comprising tetrathiomolybdic acid or a pharmaceutically acceptable salt thereof) of the disclosure, tetrathiomolybdic acid may be administered in the range of about 7.8 to 60 mg per day. In certain embodiments of the methods, uses, or compositions (e.g., comprising tetrathiomolybdic acid or a pharmaceutically acceptable salt thereof) of the disclosure, tetrathiomolybdic acid is administered in an amount of about 7.8 mg every other day (or alternatively 3.9 mg daily). In certain embodiments of the methods, uses, or compositions (e.g., comprising tetrathiomolybdic acid or a pharmaceutically acceptable salt thereof) of the disclosure, tetrathiomolybdic acid is administered in an amount of about 7.8 mg daily.

In certain embodiments of the methods, uses, or compositions (e.g., comprising tetrathiomolybdic acid or a pharmaceutically acceptable salt thereof) of the disclosure, increasing the therapeutically effective amount of tetrathiomolybdic acid during the treatment might provide additional benefits. Thus, in certain embodiments of the methods, uses, or compositions (e.g., comprising tetrathiomolybdic acid or a pharmaceutically acceptable salt thereof) of the disclosure, the therapeutically effective amount of tetrathiomolybdic acid is increased after 6 weeks (i.e., after 42 days) of treatment. For example, in certain embodiments of the methods, uses, or compositions (e.g., comprising tetrathiomolybdic acid or a pharmaceutically acceptable salt thereof) of the disclosure, the initial therapeutically effective amount of tetrathiomolybdic acid (i.e., days 1 to 42) is about 7.8 mg every other day. The increased, subsequent therapeutically effective amount of tetrathiomolybdic acid (i.e., after day 42, such as on day 43 and so on), in certain embodiments of the methods, uses, or compositions (e.g., comprising tetrathiomolybdic acid or a pharmaceutically acceptable salt thereof) of the disclosure, is about 7.8 mg daily.

In certain embodiments of the methods, uses, or compositions (e.g., comprising tetrathiomolybdic acid or a pharmaceutically acceptable salt thereof) of the disclosure, decreasing the therapeutically effective amount of tetrathiomolybdic acid during the treatment might provide additional benefits. Thus, in certain embodiments of the methods, uses, or compositions (e.g., comprising tetrathiomolybdic acid or a pharmaceutically acceptable salt thereof) of the disclosure, the therapeutically effective amount of tetrathiomolybdic acid is decreased after 6 weeks (i.e., after 42 days) of treatment. For example, in certain embodiments of the methods, uses, or compositions (e.g., comprising tetrathiomolybdic acid or a pharmaceutically acceptable salt thereof) of the disclosure, the initial therapeutically effective amount of tetrathiomolybdic acid (i.e., days 1 to 42) is about 7.8 mg daily. The decreased, subsequent therapeutically effective amount of tetrathiomolybdic acid (i.e., after day 42, such as on day 43 and so on), in certain embodiments of the methods, uses, or compositions (e.g., comprising tetrathiomolybdic acid or a pharmaceutically acceptable salt thereof) of the disclosure, is about 7.8 mg every other day.

As described above, in some embodiments of the methods, uses, or compositions (e.g., comprising tetrathiomolybdic acid or a pharmaceutically acceptable salt thereof) of the disclosure, the tetrathiomolybdic acid may be in the form of a pharmaceutically acceptable salt. “Pharmaceutically acceptable salt” refers to a salt of a compound disclosed herein, which possesses the desired pharmacological activity of the parent compound. The salt is not particularly limited. Such salts include: (1) acid addition salts, formed with inorganic acids such as hydrochioric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid. benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. For example, in some embodiments of the methods, uses, or compositions (e.g., comprising tetrathiomolybdic acid or a pharmaceutically acceptable salt thereof) of the disclosure, the tetrathiomolybdic acid is a pharmaceutically acceptable salt thereof, wherein the pharmaceutically acceptable salt is selected from bis(triethylmethyl ammonium), bis(triethylphenyl ammonium), bis(acetylcholine). bis[2-(methoxy)ethyltrimethyl ammonium], bis[alkyldimethyl(phenylmethyl) ammonium], bis(1-ethyl-3-methyl-1H-imidazolium), bis(phenyltrimethylammonium), bis(benzyltrimethylammonium), pentane-1,5-bis(trimethylammonium), bis(2-hydroxyiminomethyl-1-methyl-pyridinium), bis(1,1-dimethylpyrrolidinium), ethylene bis ammonium, bis(1,4-dimethylpyridinium), bis(vinyltrimethyl ammonium), bis(cyclopropylmethyltrimethyl ammonium), bis(benzylphenyldimethyl ammonium), hexane-1,6-bis(trimethyl ammonium), bis[(2-hydroxyethyl)trimethyl ammonium], propane-1,3-bis(trimethylammonium), tetrapropylammonium, ammonium, butane-1,4-bis(trimethylammonium), or ethylene bis(trimethylammonium). For example, in some embodiments of the methods, uses, or compositions (e.g., comprising tetrathiomolybdic acid or a pharmaceutically acceptable salt thereof) of the disclosure, the tetrathiomolybdic acid is a pharmaceutically acceptable salt thereof, wherein the pharmaceutically acceptable salt is bis-choline salt, e.g., bis-choline tetrathiomolybdate. In some embodiments of the methods, uses, or compositions (e.g., comprising tetrathiomolybdic acid or a pharmaceutically acceptable salt thereof) of the disclosure, bis-choline tetrathiomolybdate (also known as ALXN1840, BC-TTM, tiomolibdate choline, tiomolibdic acid, and WTX101) is administered in the methods, uses, or compositions of the disclosure. In some embodiments of the methods, uses, or compositions (e.g., comprising tetrathiomolybdic acid or a pharmaceutically acceptable salt thereof) of the disclosure, the pharmaceutically acceptable salt is not an ammonium salt.

TTM targets the following medical needs:
Rapid and sustained control of copper and clinical symptoms, with a low risk of neurological worsening—through the rapid formation of irreversible copper tetrathiomolybdate-protein complexes leading to a rapid copper mobilization and sequestration that could protect patients with WD from tissue toxicity including neurological deterioration.

Results from previous studies support a proposed mechanism of action of TTM whereby copper is mobilized to the bloodstream and sequestered through the formation of stable tripartite complexes (TPCs) comprising TTM, copper, and albumin.

A therapeutic option that is efficacious and well tolerated, and suitable for all naïve and pre-treated patients, including those with neurological symptoms who are at greatest risk of neurological deterioration during the initial phases of chelation therapy.

Improved compliance over long-term treatment through an improved tolerability and the convenience of a simplified dosing regimen (once daily [QD]) compared to current therapeutic options (multiple daily dosing in the fasted state).

BC-TTM has been evaluated in patients with WD in the Phase 2 Study 201 (registered with ClinicalTrials.gov, number NCT02273596; Weiss et al., 2017, Lancet Gastroenterol Hepatol. 2(12): 869-76, incorporated by reference in its entirety), which enrolled 28 patients with WD. Study 201 is described in Example 2 of U.S. Provisional Patent Application No. 63/339,307, filed May 6, 2022, and incorporated by reference herein. Final results from the main 24-week study showed that TTM monotherapy reduced mean serum cNCC_{corrected} by 72% at Week 24 compared with baseline, a significant (p<0.0001) reduction. The reduction in cNCC_{corrected} was sustained through Week 72 or longer. Initial increases in total plasma copper, exchangeable copper, and labile bound copper (LBC, mostly bound to albumin) were observed, followed by a gradual decline to baseline or even lower. These results suggest that TTM mobilizes copper from tissues to blood, forming copper-albumin-tetrathiomolybdate complexes, thereby meeting the treatment goal of a therapy for WD that removes excessive copper from tissues such as the liver.

TTM treatment also resulted in significant improvements in neurological status (p<0.0001) and patient-reported disability (p<0.001) measured as a change from baseline in Unified WD Rating Scale (UWDRS) Part III and Part II, respectively. In the Extension Period of Study 201, 48-week follow up data indicate maintained overall improvement in disability as shown by mean reduction in the UWDRS Part II score and maintained overall improvement in neurologic status as shown by mean reduction in UWDRS Part III.

In addition, liver status, as measured by the Modified Nazer Score, was stabilized or improved in the majority of patients. Treatment with TTM was generally well-tolerated, with most reported AEs being mild (Grade 1) to moderate (Grade 2). The most frequently reported drug-related AEs were changes in hematological parameters, fatigue, sulphur eructations, and other gastrointestinal symptoms. Reversible liver function test elevations were observed in 39% of patients; these elevations were mild to moderate, asymptomatic, were associated with no notable increases in bilirubin, and normalized with dose reduction or treatment interruption. No paradoxical neurological worsening was observed upon treatment initiation with TTM. All patients who completed the 24-week Study Period were enrolled in a 36-month Extension Period. Preliminary available follow-up data at 48 weeks from the ongoing 36-month Extension Period of the study were consistent with the 24-week Study Period results.

In patients with WD, the most commonly reported AEs associated with multiple TTM dosing are reversible, dose-dependent, liver test elevations (transaminases) observed after treatment initiation with doses of 30 mg per day or higher. In the Phase 2 Study 201, reversible liver test elevations were observed in 39% of patients. These elevations were generally mild to moderate, asymptomatic and normalized with dose adjustments. No initial drug-induced neurological worsening was observed upon treatment initiation with TTM. As tetrathiomolybdate is a copper modulating agent, there is the risk of producing copper deficiency with prolonged dosing with TTM. Changes in hematological parameters (thrombocytopenia and leukopenia), attributed by the Investigators to overtreatment and resultant copper deficiency, have been observed. Multiple-dose WD studies, like Study 301, therefore, involve frequent monitoring for these potential adverse hematological and hepatic effects of TTM.

Based on the efficacy results from Study 201 (lowering of free copper, stabilization or improvement of liver status and improvement in neurological symptoms) and the risk mitigation measures included in the protocol to account for the most common AEs reported in the study, TTM is considered to have an acceptable benefit/risk in adult patients. The pathophysiology of copper overload does not differ substantially between adolescents and adults with WD, and the approved treatment options and therapeutic goal of copper control are also the same for adolescents and adults.

In some embodiments of the methods, uses, or compositions (e.g., comprising tetrathiomolybdic acid or a pharmaceutically acceptable salt thereof) of the disclosure as described herein, the administering of the therapeutically effective amount of bis-choline tetrathiomolybdate is for at least 24 weeks, for at least 36 weeks, or for at least 48 weeks. In certain embodiments of the methods, uses, or compositions (e.g., comprising tetrathiomolybdic acid or a pharmaceutically acceptable salt thereof) of the disclosure, the therapeutically effective amount of bis-choline tetrathiomolybdate is administered to the subject in a fasted state.

A therapeutically effective amount of BC-TTM has been previously established. For example, in certain embodiments of the methods, uses, or compositions (e.g., comprising BC-TTM) of the disclosure, BC-TTM may be administered in the range of about 7.5 to 60 mg per day, such as 15 to 60 mg per day. In certain embodiments of the methods, uses, or compositions (e.g., comprising BC-TTM) of the disclosure, BC-TTM is administered in an amount of about 15 mg every other day (or alternatively 7.5 mg daily). In certain embodiments of the methods, uses, or compositions (e.g., comprising BC-TTM) of the disclosure, BC-TTM is administered in an amount of about 15 mg daily. In certain embodiments, BC-TTM is administered in an amount of about 30 mg daily (e.g., about 15 mg taken twice daily or two 15 mg tablets taken once daily). In certain embodiments, BC-TTM is administered in an amount of about 45 mg daily (e.g., about 15 mg taken trice daily or three 15 mg tablets taken once daily). In certain embodiments, BC-TTM is administered in an amount of about 60 mg daily (e.g., about 15 mg taken four times daily or four 15 mg tablets taken once daily).

In certain other embodiments of the methods, uses, or compositions (e.g., comprising tetrathiomolybdic acid or a pharmaceutically acceptable salt thereof) of the disclosure, BC-TTM may be administered in the range of about 15 to 60 mg every other day. In certain embodiments of the methods, uses, or compositions (e.g., comprising BC-TTM) of the disclosure, BC-TTM is administered in an amount of about 15 mg every other day. In certain embodiments, BC-TTM is administered in an amount of about 30 mg every other day. In certain embodiments, BC-TTM is administered in an amount of about 45 mg every other day. In certain embodiments, BC-TTM is administered in an amount of about 60 mg every other day.

In certain embodiments of the methods, uses, or compositions (e.g., comprising tetrathiomolybdic acid or a pharmaceutically acceptable salt thereof) of the disclosure increasing the therapeutically effective amount of BC-TTM during the treatment might provide additional benefits. Thus, in certain embodiments of the methods, uses, or compositions (e.g., comprising tetrathiomolybdic acid or a pharmaceutically acceptable salt thereof) of the disclosure, the therapeutically effective amount of BC-TTM is increased after 6 weeks (i.e., after 42 days) of treatment. For example, in certain embodiments of the methods, uses, or compositions (e.g., comprising tetrathiomolybdic acid or a pharmaceutically acceptable salt thereof) of the disclosure, the initial therapeutically effective amount of BC-TTM (i.e., days 1 to 42) is about 15 mg daily. The increased, subsequent therapeutically effective amount of BC-TTM (i.e., after day 42, such as on day 43 and so on), in certain embodiments, is about 30 mg daily. In certain embodiments, the increased subsequent therapeutically effective amount of BC-TTM is about 45 mg daily. In certain embodiments, the increased subsequent therapeutically effective amount of BC-TTM is about 60 mg daily. For example, in certain other embodiments, the initial therapeutically effective amount of BC-TTM is about 30 mg daily. The increased, subsequent therapeutically effective amount of BC-TTM, in certain embodiments, is about 45 mg daily. In certain embodiments, the increased subsequent therapeutically effective amount of BC-TTM is about 60 mg daily. For example, in certain embodiments of the methods, uses, or compositions (e.g., comprising tetrathiomolybdic acid or a pharmaceutically acceptable salt thereof) of the disclosure, the initial therapeutically effective amount of BC-TTM is about 15 mg every other day. The increased, subsequent therapeutically effective amount of BC-TTM, in certain embodiments of the methods, uses, or compositions (e.g., comprising tetrathiomolybdic acid or a pharmaceutically acceptable salt thereof) of the disclosure, is about 15 mg daily.

In certain embodiments of the methods, uses, or compositions (e.g., comprising tetrathiomolybdic acid or a pharmaceutically acceptable salt thereof) of the disclosure, decreasing the therapeutically effective amount of BC-TTM during the treatment might provide additional benefits. Thus, in certain embodiments of the methods, uses, or compositions (e.g., comprising tetrathiomolybdic acid or a pharmaceutically acceptable salt thereof) of the disclosure, the therapeutically effective amount of BC-TTM is decreased after 6 weeks (i.e., after 42 days) of treatment. For example, in certain embodiments, the initial therapeutically effective amount of BC-TTM (i.e., days 1 to 42) is about 60 mg daily. The decreased, subsequent therapeutically effective amount of BC-TTM (i.e., after day 42, such as on day 43 and so on), in certain embodiments, is about 45 mg daily. In certain embodiments, the decreased subsequent therapeutically effective amount of BC-TTM is about 30 mg daily. In certain embodiments, the decreased subsequent therapeutically effective amount of BC-TTM is about 15 mg daily. For example, in certain other embodiments, the initial therapeutically effective amount of BC-TTM is about 30 mg daily.

The decreased, subsequent therapeutically effective amount of BC-TTM, in certain embodiments, is about 15 mg daily. For example, in certain other embodiments of the methods, uses, or compositions (e.g., comprising tetrathiomolybdic acid or a pharmaceutically acceptable salt thereof) of the disclosure, the initial therapeutically effective amount of BC-TTM is about 15 mg daily. The decreased, subsequent therapeutically effective amount of BC-TTM, in certain embodiments of the methods, uses, or compositions (e.g., comprising tetrathiomolybdic acid or a pharmaceutically acceptable salt thereof) of the disclosure, is about 15 mg every other day.

In some embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to inhibit progression of neurological damage of the subject, wherein the subject who had an incomplete (or inadequate) response or is intolerant to standard of care (SoC) treatment. In some embodiments of the methods, uses, or compositions (e.g., comprising tetrathiomolybdic acid or a pharmaceutically acceptable salt thereof) of the disclosure as described herein the therapeutically effective amount of tetrathiomolybdic acid or a pharmaceutically acceptable salt thereof is sufficient to inhibit progression of liver damage of the subject, wherein the subject had an incomplete (or inadequate) response or is intolerant to SoC treatment.

In some embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve or maintain one or more of neurological symptoms, psychiatric symptoms, clinical symptoms, disability status, or treatment satisfaction of the subject, wherein the subject had an incomplete (or inadequate) response or is intolerant to SoC treatment. In certain embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve one or more of neurological symptoms, psychiatric symptoms, clinical symptoms, disability status, or treatment satisfaction of the subject, wherein the subject had an incomplete (or inadequate) response or is intolerant to SoC treatment. For example, in some embodiments of the methods, uses, or compositions (e.g., comprising tetrathiomolybdic acid or a pharmaceutically acceptable salt thereof) of the disclosure as described herein, the improvement is observed after at least 24 weeks, at least 36 weeks, or at least 48 weeks of administering the therapeutically effective amount of tetrathiomolybdic acid or a pharmaceutically acceptable salt thereof.

In some embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve a Unified Wilson Disease Rating Scale (UWDRS) Part II total score or a Unified Wilson Disease Rating Scale (UWDRS) Part III total score of the subject, wherein the subject had an incomplete (or inadequate) response or is intolerant to SoC treatment. In certain embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve a Unified Wilson Disease Rating Scale (UWDRS) Part II total score and a Unified Wilson Disease Rating Scale (UWDRS) Part III total score of the subject, wherein the subject had an incomplete (or inadequate) response or is intolerant to SoC treatment. For example, in some embodiments of the methods, uses, or compositions (e.g., comprising tetrathiomolybdic acid or a pharmaceutically acceptable salt thereof) of the disclosure, the therapeutically effective amount of tetrathiomolybdic acid or a pharmaceutically acceptable salt thereof is sufficient to improve the UWDRS Part II Total Score by at least 5%, at least 10%, at least 15%, or at least 20% of the subject, wherein the subject had an incomplete (or inadequate) response or is intolerant to SoC treatment. In some embodiments of the methods, uses, or compositions (e.g., comprising tetrathiomolybdic acid or a pharmaceutically acceptable salt thereof) of the disclosure, the therapeutically effective amount of tetrathiomolybdic acid or a pharmaceutically acceptable salt thereof is sufficient to improve the UWDRS Part III Total Score by at least 5%, at least 10%, at least 15%, or at least 20% of the subject, wherein the subject had an incomplete (or inadequate) response or is intolerant to SoC treatment. In some embodiments of the methods, uses, or compositions (e.g., comprising tetrathiomolybdic acid or a pharmaceutically acceptable salt thereof) of the disclosure, the improvement in the UWDRS Part II Total Score and/or the UWDRS Part III Total Score is observed after at least 24 weeks, at least 36 weeks, or at least 48 weeks of administering the therapeutically effective amount of tetrathiomolybdic acid or a pharmaceutically acceptable salt thereof.

In some embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve one or more neurological symptoms of the subject (e.g., a subject who had an incomplete (or inadequate) response or is intolerant, as described herein) as measured according to Unified Wilson Disease Rating Scale (UWDRS) Part II and/or Part III, the one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor. For example, in some embodiments of the methods, uses, or compositions (e.g., comprising tetrathiomolybdic acid or a pharmaceutically acceptable salt thereof) of the disclosure, the improvement according to Unified Wilson Disease Rating Scale (UWDRS) Part II and/or Part III is observed after at least 24 weeks, at least 36 weeks, or at least 48 weeks of administering the therapeutically effective amount of tetrathiomolybdic acid or a pharmaceutically acceptable salt thereof.

In some embodiments of the methods, uses, or compositions (e.g., comprising tetrathiomolybdic acid or a pharmaceutically acceptable salt thereof) of the disclosure as described herein, the therapeutically effective amount of tetrathiomolybdic acid or a pharmaceutically acceptable salt thereof is sufficient to improve one or more neurological symptoms of the subject (e.g., a subject who had an incomplete (or inadequate) response or is intolerant, as described herein) as measured according to Unified Wilson Disease Rating Scale (UWDRS) Part II and/or Part III, the one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest. For example, in some embodiments of the methods, uses, or compositions (e.g., comprising tetrathiomolybdic acid or a pharmaceutically acceptable salt thereof) of the disclosure, the improvement according to Unified Wilson Disease Rating Scale (UWDRS) Part II and/or Part III is observed after at least 24 weeks, at least 36 weeks, or at least 48 weeks of administering the therapeutically effective amount of tetrathiomolybdic acid or a pharmaceutically acceptable salt thereof.

In some embodiments of the methods, uses, or compositions (e.g., comprising tetrathiomolybdic acid or a pharmaceutically acceptable salt thereof) of the disclosure as described herein, the method, use, or composition comprising tetrathiomolybdate acid or a pharmaceutically acceptable salt thereof further includes evaluating the subject (e.g., a subject who had an incomplete (or inadequate) response or is intolerant, as described herein) for improvements in disability and neurologic symptoms as measured according to Unified Wilson Disease Rating Scale (UWDRS) Part II and/or Part III. In some embodiments of the methods, uses, or compositions (e.g., comprising tetrathiomolybdic acid or a pharmaceutically acceptable salt thereof) of the disclosure as described herein, the method, use, or composition comprising tetrathiomolybdic acid or a pharmaceutically acceptable salt thereof further includes evaluating the subject (e.g., a subject who had an incomplete (or inadequate) response or is intolerant, as described herein) for improvements in disability status, psychiatric symptoms, clinical symptoms, treatment satisfaction, or a combination thereof.

In certain embodiments of the methods, uses, or compositions (e.g., comprising tetrathiomolybdic acid or a pharmaceutically acceptable salt thereof) of the disclosure, when values are expressed as approximations, by use of the antecedent “about,” it will be understood that the particular value forms one possible embodiment of the methods, uses, or compositions (e.g., comprising tetrathiomolybdic acid or a pharmaceutically acceptable salt thereof) of the disclosure and variation of the given value is possible (e.g., about 80 may include 80±10%). It will be further understood that the endpoints of each of the ranges are significant both in relation to the other endpoint, and independently of the other endpoint.

As used herein, “total copper” refers to the sum of all copper species in blood (for example, in serum or plasma). Total copper includes both ceruloplasmin (Cp)-bound copper and all species of non-ceruloplasmin bound copper. In general, total copper may be directly measured with high sensitivity and specificity by mass-spectroscopy, such as inductively coupled plasma-mass spectrometry (ICP-MS).

The term “NCC” refers to the fraction of total copper that is not bound to ceruloplasmin (i.e., “non-ceruloplasmin-bound copper”). Under commonly used estimation methods, NCC is estimated using direct measurements of total copper and Cp in the blood (such as, e.g., serum or plasma) and the following formula:

$$NCC ( μ M ) = \frac{PTC ( μ g / L ) - ( 3.15 \times ceruloplasmin ( mg / L ) )}{63.5 ( μ g / μ mol )} .$$

The term “cNCC” refers to NCC as calculated using this formula. The calculation is premised on an assumption that six copper atoms are always bound to a single Cp molecule, and that NCC and ceruloplasmin concentrations are directly correlated. In reality, Cp may show considerable heterogeneity in the number of copper atoms associated per Cp molecule. This formula assumes that six copper atoms bind per one Cp molecule, but the copper/Cp ratio varies with disease state. In fact, 6-8 copper atoms can actually bind to Cp, and in WD usually fewer than six copper atoms are associated per Cp molecule.

In subjects treated with TTM, non-ceruloplasmin-bound copper includes the fraction of total copper that is bound to albumin, transcuprein, and other less abundant plasma proteins (collectively referred to as LBC) or in tetrathiomolybdate-Cu-albumin tripartite complexes (TPCs). The concentration of TPCs cannot be directly measured, but in certain embodiments of the methods, uses, or compositions (e.g., comprising tetrathiomolybdic acid or a pharmaceutically acceptable salt thereof) of the disclosure, the concentration of TPCs may be estimated using molybdenum concentration as a surrogate.

The term “NCC_{corrected} refers to the fraction of total copper that is not bound to ceruloplasmin or in a TPC (i.e., LBC) and which is calculated by subtracting a direct measure of molybdenum in the blood (such as, e.g., serum or plasma) from the estimated NCC (or cNCC). “NCC_{corrected}” is thus a correction of the cNCC value to account for the presence of molybdenum-copper-albumin tripartite complexes in the blood of TTM-treated subjects.

The term “dNCC” refers to NCC as directly measured using an NCC assay. For example, in certain embodiments of the methods, uses, or compositions (e.g., comprising tetrathiomolybdic acid or a pharmaceutically acceptable salt thereof) of the disclosure, dNCC is directly measured using the NCC assay as disclosed in PCT Patent Application Publication No. WO2021/050850, filed on Sep. 11, 2020, herein incorporated by reference in its entirety.

The terms “LBC” or “labile-bound copper” refer to the fraction of total copper which is bound to albumin, transcuprein, and other less abundant plasma proteins. LBC thus comprises the fraction of total copper which is not bound to either ceruloplasmin or TPCs. In certain embodiments of the methods, uses, or compositions (e.g., comprising tetrathiomolybdic acid or a pharmaceutically acceptable salt thereof) of the disclosure, the LBC fraction is directly measured using an LBC assay. For example, in certain embodiments, the LBC assay is as disclosed in PCT Patent Application Publication No.

WO2021/050850, filed on Sep. 11, 2020, herein incorporated by reference in its entirety. In a biological sample in which no TPC is present, the NCC and the LBC fractions are the same. The methods and uses of the disclosure are illustrated further by the following Examples, which are not to be construed as limiting the disclosure in scope or spirit to the specific procedures and compounds described therein.

### Example 1

### Overall Design

Study 301 (registered with ClinicalTrials.gov, number NCT03403205) was conducted to assess the efficacy and safety of BC-TTM versus standard of care (SoC) in patients with Wilson disease (WD) who were 12 years of age or older, or 18 years and older. Currently available drugs have high rates of treatment discontinuation due to tolerability and efficacy issues. They also need to be dosed 2 to 4 times per day and must be taken in the fasted state. Their AE profiles and complicated dosing regimens lead to poor treatment compliance and high rates of treatment failure, a major concern in WD, which is a disease that requires life-long treatment.

Unlike currently available treatments for WD, TTM is designed to provide an alternative copper-protein transport mechanism, and rapidly form copper-protein complexes with very high specificity for copper to quickly treat the underlying disease by mobilizing excess tissue copper.

| | |
|---|---|
| Objectives | Endpoints |

Primary

| | |
|---|---|
| To evaluate the efficacy of BC-TTM | Daily mean area under the effect- |
| administered for 48 weeks, | time curve (AUEC) of directly |
| compared to standard of care (SoC), | measured non-ceruloplasmin-bound |
| on copper control in WD patients | copper (dNCC) from baseline to 48 |
| aged 12 years and older | weeks |

Secondary

| | |
|---|---|
| Establish the safety and tolerability | Incidence of |
| of individualized dosing of BC-TTM | Adverse events/serious adverse |
| | events (AE/SAEs) from baseline |
| | through 48 weeks (Primary Period); |
| | also Extension Period: baseline |
| | through up to 60 months |
| | Clinical laboratory test data |
| | Neurological and physical |
| | examination findings |
| | 12-lead electrocardiogram (ECG) data |
| | Vital signs |
| Evaluate the effects of BC-TTM on | Change from baseline in the Unified |
| disability status | Wilson disease Rating Scale |
| | (UWDRS) Part II total score through |
| | 48 weeks (Primary Period); also |
| | Extension Period: baseline through |
| | up to 60 months |
| Evaluate the effects of BC-TTM on | Change from baseline in UWDRS |
| neurological status | Part III total score and individual |
| | item/subscales (arising from a chair, |
| | gait, handwriting, and speech) |
| | through 48 weeks (Primary Period); |
| | also Extension Period: baseline |
| | through up to 60 months |
| Evaluate the global effects of BC- | Clinical Global Impression- |
| TTM on global clinical symptoms | Improvement Scale (CGI-I) through |
| | 48 weeks (Primary Period); also |
| | Extension Period: baseline through |
| | up to 60 months |
| | Change from baseline in Clinical |
| | Global Impression-Severity Scale |
| | (CGI-S) through 48 weeks (Primary |
| | Period); also Extension Period: |
| | baseline through up to 60 months |
| Evaluate the efficacy of BC-TTM | Absolute change from baseline (Day |
| administered for 48 weeks, | 1) to 48 weeks in calculated NCC |
| compared to SoC, on copper control | (cNCC) in plasma. |
| in WD patients aged 12 years and | Percentage change from baseline in |
| older. | cNCC in plasma. |
| | For BC-TTM-treated patients, the cNCC in |
| | plasma will be corrected for the amount of |
| | copper bound to the BC-TTM tripartite |
| | complex (TPC) (cNCC_{corrected}) |
| Evaluate the effects of BC-TTM on | cNCC responder rate at baseline |
| the cNCC responder rate | through 48 weeks (Primary Period); |
| | also Extension Period: baseline |
| | through up to 60 months |
| Explore other directly measured | Plasma total copper, plasma |
| pharmacodynamics (PD) and | ultrafiltrate (PUF)-copper, dNCC, |
| biomarkers of BC-TTM | labile bound copper (LBC), |
| | ceruloplasmin (Cp), and |
| | ceruloplasmin-bound copper (CpC) |
| | concentration-time profiles in |
| | plasma |
| | Daily mean AUEC of plasma dNCC |
| | (from 0 to 24 and 24 to 48 weeks) |
| | Daily mean AUEC of LBC from 0 to |
| | 48 weeks |
| | Daily mean AUEC of plasma total |
| | copper from 0 to 48 weeks |
| | Absolute and percentage change |
| | from baseline (Day 1) to 48 weeks |
| | in dNCC |
| | Absolute and percentage change |
| | from baseline (Day 1) to 48 weeks |
| | in LBC |
| Explore BC-TTM effect on initial | Time to first confirmed increase in |
| sequestering and decoppering | plasma dNCC and total copper |
| phase compared to SoC based on | concentration |
| directly measured pharmacokinetic | Time to minimum and maximum |
| (PK)/PD and biomarkers | concentration of: |
| | Plasma total copper |
| | Plasma dNCC |
| | Plasma LBC |
| | Ratio plasma dNCC:total copper |
| | Ratio plasma LBC:total copper |
| | Urinary molybdenum |
| | Ratio urinary molybdenum:copper |
| | Ratio urinary molybdenum:dosed molybdenum |
| | Plasma Cp |
| | Plasma CpC |
| | Ratio plasma Cp:total copper |
| | Ratio plasma CpC:total copper |
| | Ratio plasma CpC:Cp |
| Explore BC-TTM effect on | Time for return to pre-dose baseline |
| subsequent maintenance phase | for the following measures: |
| compared to SoC based on directly | Plasma total copper |
| measured PK/PD and biomarkers | Plasma dNCC |
| | Plasma LBC |
| | Ratio plasma dNCC:total copper |
| | Ratio plasma LBC:total copper |
| | Urinary molybdenum |
| | Ratio urinary molybdenum:copper |
| | Ratio urinary molybdenum:dosed molybdenum |
| | Plasma Cp concentration |
| | Plasma CpC concentration |
| | Ratio plasma Cp:total copper |
| | Ratio plasma CpC:total copper |
| | Ratio plasma CpC:Cp |
| Explore PK of BC-TTM | Total molybdenum and PUF- |
| | molybdenum concentration-time |
| | profiles in plasma |
| Evaluate the effects of BC-TTM on | LBC responder rate at 48 weeks |
| the LBC responder rate | |
| Explore the change in 24-hour | Change from baseline to 48 weeks |
| urinary copper and urinary | in 24-hour urinary copper and |
| molybdenum | urinary molybdenum |

Abbreviations:
AE = adverse event;
AESI = adverse event of special interest;
AUEC = area under the effect-time curve;
CGI-I = Clinical Global Impression-Improvement Scale;
CGI-S = Clinical Global Impression-Severity Scale;
Cp = ceruloplasmin;
CpC = ceruloplasmin-bound copper;
ECG = electrocardiogram
LBC = labile bound copper;
(d)(c)NCC = (directly measured) (calculated) non-ceruloplasmin-bound copper;
cNCC_{corrected} = calculated corrected NCC;
PD = pharmacodynamics;
PK = pharmacokinetics;
PUF = plasma ultrafiltrate;
SAE = serious adverse event;
SoC = standard of care;
TPC = tripartite complex;
UWDRS = Unified Wilson Disease Rating Scale

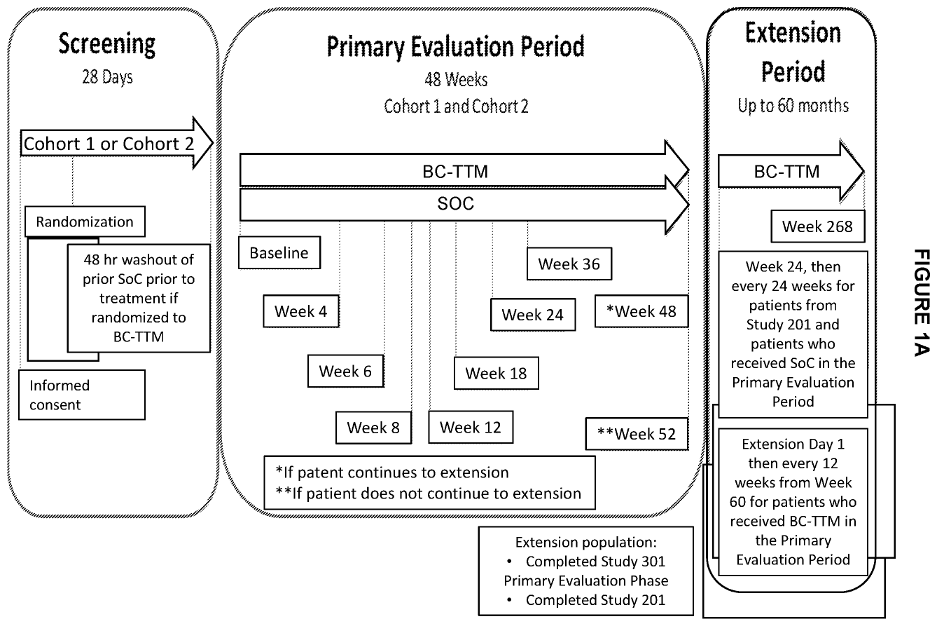

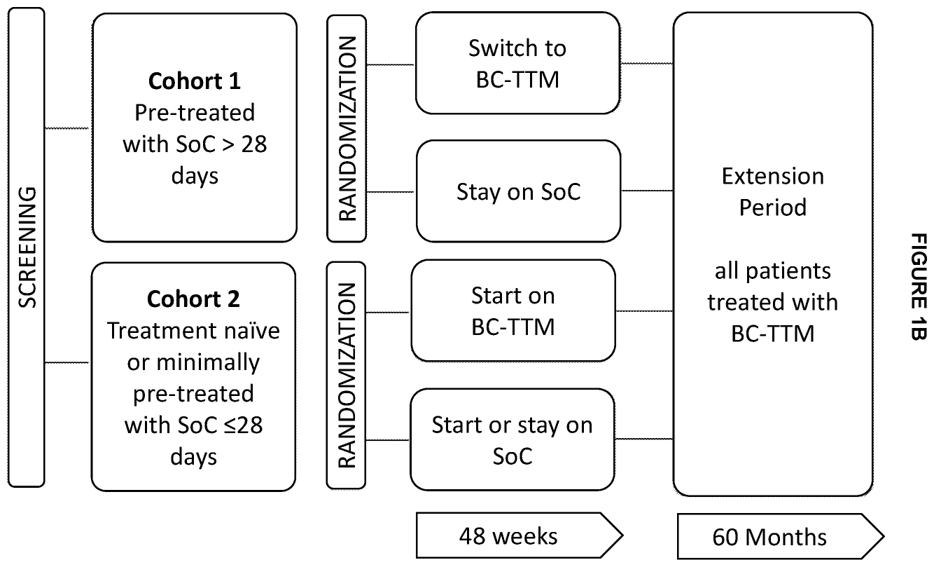

FIG. 1 provides a schematic view of the study design. This was a randomized, rater-blinded, multi-center study assessing the efficacy and safety of TTM versus standard of care (SoC). This study is referred throughout as Study 301. In the Primary Evaluation Period, efficacy and safety was assessed for an individualized TTM dosing regimen compared with SoC administered for 48 weeks in patients with WD who are aged 12 years and older, or 18 years and older.

Patients meeting all inclusion and no exclusion criteria were enrolled into the study and studied as outpatients. Eligible patients with WD were enrolled into 1 of 2 cohorts.
Cohort 1: Patients who had received SoC therapy (i.e., chelation therapy with penicillamine or trientine, zinc therapy, or a combination of both chelation and zinc therapy) for >28 days
Cohort 2: Patients who were treatment-naïve or who had received SoC therapy for ≤28 days
   All patients were enrolled by cohort in a 3:1 ratio and be randomized within cohort in a 2:1 ratio to treatment with TTM or SoC (either as continued therapy in Cohort 1 or as continued or initial therapy in Cohort 2). Treatments were assigned randomly, stratified by cohort, utilizing an interactive voice/web response system (FIG. 1B).

Patients who were randomized to receive TTM were required to withhold treatment with SoC for ≥48 hours immediately prior to first study assessment on Day 1. Patients who were randomized to TTM received BC-TTM as delayed-release tablets for oral administration at doses ranging from 15 mg every other day (QOD) to 60 mg QD. Efficacy and safety assessments were performed at scheduled visits, while AEs and concomitant medications were monitored continuously throughout the study. Patients randomized to SoC initiated treatment or continued treatment on their current regimen where possible, without compromising the safety of individual patients.

The Primary Evaluation Period consisted of an up to 28-day Screening Period, a 1-day Enrollment Visit, a 48-week Treatment Period, and a Follow-up Visit 4 weeks after the last dose for patients who do not elect to continue in the Extension Period.

Patients in Study 301 who completed the 48-week Treatment Period were offered the opportunity to participate in an up to 60-month Extension Period to evaluate the long-term safety and efficacy of TTM.

Control of exchangeable copper is important for management of hepatic and neuropsychiatric manifestations in patients with WD. Results from related studies support a proposed mechanism of action of TTM whereby copper is mobilized to the bloodstream and sequestered through the formation of stable TPCs, with a low risk for neurologic worsening due to copper exchange from chelators with a lower affinity for copper. Study 301 is the first prospective, randomized study to compare tetrathiomolybdate with penicillamine, trientine or zinc in WD.

The primary endpoint integrated copper mobilization and sequestration (i.e., copper control) throughout the 48-week Primary Treatment Period by assessment of daily mean area under the effect-time curve (AUEC) of directly measured non-ceruloplasmin-bound copper (dNCC) concentration. AUEC characterizes and measures the cumulative effect of TTM. Measurement of dNCC in plasma is highly sensitive and accurate, requiring no special formulas or assumptions. As proposed, dNCC AUEC_{0-48 W} is an indirect measure of copper mobilization and sequestration. Results from a related study, 201, were strongly suggestive of rapid onset of TPC formation within hours following TTM administration and potent mobilization of excess copper from tissue into plasma persisting through Week 12, which appeared nearly complete by Week 24. Over the course of 24-48 weeks of treatment with TTM in Study 201, the plasma dNCC concentration continued to gradually decrease to baseline. The gradual, sustained reduction of plasma dNCC concentrations overtime indicates that copper mobilized by TTM is not simply redeposited back into the tissue, as both plasma concentrations of tetrathiomolybdate (from a daily weighted average dose of 30 mg BC-TTM administration) and circulating bodily albumin (the other 2 components of TPC) remained essentially constant and sufficiently available for TPC formation throughout the study. Thus, daily mean dNCC AUEC_{0-48 W} is suitable as a quantitative measure of TTM treatment effect in WD based on its mechanism of action.

Measurement of the AUEC for dNCC as the primary outcome measure overcomes many of the limitations of the estimated cNCC approach. Calculated estimates of NCC rely on separate measurements of plasma total copper and ceruloplasmin protein. The amount of copper within Cp is further estimated based on an assumed ratio of 6 copper atoms per molecule of Cp, which may be an overestimate in WD. Overestimation of ceruloplasmin-bound copper (CpC) results in approximately 20% of samples yielding physiologically impossible negative values for cNCC. In patients treated with TTM, estimation of cNCC requires additional correction for the presence of copper in the TPC. TPC copper cannot be measured directly but must instead be estimated based on the plasma concentration of molybdenum.

For the primary evaluation of efficacy and safety, a treatment period of 1 year was chosen to allow sufficient time for evaluation of changes in biochemical measures of copper, as well as changes in liver function and neurologic disability and an adequate evaluation of safety and tolerability.

Following the Primary Evaluation Period, patients randomized to SoC during the Primary Evaluation Period switched to treatment with TTM in the Extension Period, providing further evaluation of changes in copper, liver function and neurologic dysfunction. The Extension Period of up to 5 additional years will allow further evaluation of the long-term efficacy, safety, tolerability, and clinical outcomes in patients treated with TTM.

The UWDRS scoring system was developed specifically for the motor and movement disorders associated with chronic copper neurotoxicity in WD. Some individual items/subscales of the UWDRS Part III (arising from a chair, gait, handwriting, and speech) in addition to the UWDRS Parts I, II, and III overall, were assessed to further define the range of burdensome signs and symptoms of WD, so as to further understand the assessment of treatment effects on patients with WD. The UWDRS scores for consciousness (Part I) and abnormal neurologic examination findings (Part III) will be determined by a trained neurologist rater who is blinded to study treatment randomization. These scores were used to provide a rigorous dataset for evaluating changes from baseline.

Inclusion of adolescents≥12 years in this protocol is justified by the natural history of WD. In a large European cohort of 1357 patients, the average age at diagnosis of WD was 19.8 years, and half of all patients were diagnosed before the age of 18. Compared with adults, children and adolescents are more likely to present with hepatic symptoms rather than neuropsychiatric manifestations. The goal of treatment in adolescents is the same as in adults, namely prompt and effective removal of excess copper from tissues.

### Drug Dosing

BC-TTM daily dosing intended for use in this study was based on the doses established as safe and effective in the previous WD studies performed with BC-TTM. Daily doses of 30 to 60 mg have been shown as effective in de-coppering newly diagnosed patients with WD or maintaining a normal copper level in patients with WD previously treated with SoC. In patients with WD treated with TTM, asymptomatic elevation hepatic transaminases and/or gamma glutamyltransferase were seen in 39% of patients. Elevation of liver enzymes was dose-dependent and reversible with interruption or dose reduction of TTM. Therefore, the dose of BC-TTM in the current study started at 15 mg daily (i.e., 7.8 mg TTM daily) and was limited to a maximum of 60 mg daily, the highest dose studied and considered to have a good safety profile in healthy volunteers. The intent was to individually titrate the dose of BC-TTM, as is done with the currently available chelators, to an appropriate dose based on cNCC levels adjusted for molybdenum plasma concentration, hematology values, and liver function tests. The dosing regimen for BC-TTM, therefore, included the following features: initial dosing QD as described below; and up titration design and individualized dosing as indicated by neurological and liver function testing.

In line with currently available WD treatments, the dose of BC-TTM was adjusted in individual patients, depending on clinical response and safety, as appropriate, based on protocol specified guidelines. A detailed dosing guide for BC-TTM dose modifications is outlined below and in Table 1.

To the extent possible, without compromising the safety of individual patients, the type and dose of SoC medication was not changed throughout the 48-week study period.

BC-TTM was supplied as white, round, delayed-release tablets for oral administration. Each tablet contained 15 mg of the bis-choline salt of tetrathiomolybdate, bis[2-hydroxyethyl) trimethyl-ammonium]tetrathiomolybdate, and the following excipients: tribasic calcium phosphate, sodium carbonate, sodium starch glycolate, and magnesium stearate. The tablets were coated with an inner pre-coat (Opadry 03K19229 clear) and outer enteric coat (Acryl-EZE white). Tablets were debossed on 1 side with a hexagon.

BC-TTM was supplied in treatment kits containing 28 tablets. The treatment kit consisted of thermoform blister strips mounted into a cardboard wallet.

BC-TTM was administered on Day 1. BC-TTM was administered orally at doses ranging from 15 mg QOD to 60 mg QD. BC-TTM was administered QD or QOD in the fasted state (1 hour before or 2 hours after meals).

Individualized TTM dosing was utilized throughout the study based on the following parameters:
Clinical criteria: dose-titration based on hepatic and neurological status;
NCC_{corrected}: dose-titration instructed based on cNCC levels adjusted for the amount of copper bound to the TTM TPC;
Safety monitoring: dose modification criteria were based on regularly scheduled assessments for recognized hematological effects of Cu lowering, hepatic testing, and neurological test.

In all patients, BC-TTM was administered at a 15 mg (equivalent to 7.8 mg TTM) QD starting dose on Day 1 continuing for the first 4 weeks. After 4 weeks, up-titration to 30 mg BC-TTM QD could be performed at the discretion of the Investigator, if the disease was not adequately controlled, taking into account the patient's clinical status and free blood copper levels, as measured by cNCC/cNCC_{corrected}, and none of the Dose Modification Criteria apply. Further dose increases were possible at the discretion of the Investigator in 15 mg BC-TTM increments at least 4 weeks apart following the same aforementioned criteria. The dose should have been lowered or interrupted if any of the relevant Dose Modification Criteria were met.

When cNCC_{corrected} levels have fallen to within the normal range (<2.3 μmol/L), and/or the clinical status of the patient was stable or improved for 2 consecutive study visits, TTM dosage may be maintained or reduced at the discretion of the Investigator. To avoid over-treatment, the dose may be reduced at any time, at the discretion of the Investigator, guided by the following: if the patient's clinical status indicates possible over-treatment and/or cNCC/cNCC_{corrected} values were below the normal range. Specific criteria for dose modification of BC-TTM were detailed in Table 1.

To the extent possible, without compromising the safety of individual patients, the type of SoC medication should not be changed throughout the 48-week study period, unless required as part of the treatment (e.g., if a patient initiates SoC at the start of the study). Similarly, to the extent possible, without compromising the safety of individual patients, the dosing of the SoC medication should remain consistent throughout the 48-week study period, unless required as part of the treatment (e.g., titration of SoC initiated at the start of the study).

TABLE 1
BC-TTM Dose Modifications for Individual Patients

| | | | Action With BC-TTM | Changes in Safety | |
|---|---|---|---|---|---|
| Test | Result | Conditions | Dosing | Monitoring^{a} | Re-Challenge^{b, c, d} |
| ALT | >5 × ↑ from | ALT above normal | Temporary interruption | Contact patient within 48 | At 15 mg QOD when |
| | baseline | range at baseline | | hours to arrange repeat | ALT <2 × ↑ from |
| | | | | testing | baseline. |
| | | | | Weekly repeat testing | |
| | >5 × ULN | ALT within normal | Temporary interruption | Contact patient within 48 | At 15 mg QOD when |
| | | range at baseline | | hours to arrange repeat | ALT <2 × ULN. |
| | | | | testing | |
| | | | | Weekly repeat testing | |
| | >2 × ↑ from | ALT above normal | Reduce dose to previous dose | Weekly repeat testing | Not applicable. |
| | baseline | range at baseline | level if up-titration has | | |
| | | | occurred or reduce dose to 15 | | |
| | | | mg QOD if on 15 mg QD. No | | |
| | | | further dose ↑ until resolution | | |
| | | | of abnormality. | | |
| | >2 × ULN | ALT within normal | Reduce dose to previous dose | Weekly repeat testing | Not applicable. |
| | | range at baseline | level if up-titration has | | |
| | | | occurred or reduce dose to 15 | | |
| | | | mg QOD if on 15 mg QD. No | | |
| | | | further dose ↑ until resolution | | |
| | | | of abnormality. | | |
| Hemoglobin | <8 g/dL in the | None | Temporary interruption | Weekly repeat testing | At 15 mg QOD when |
| | absence of | | | | hemoglobin and other |
| | bleeding | | | | hematology parameters |
| | | | | | (neutrophils and platelets) |
| | | | | | were at baseline level. |
| | >30% ↓ from | None | Reduce dose to previous dose | Weekly repeat testing | Not applicable. |
| | baseline | | level if up-titration has | | |
| | | | occurred or reduce dose to 15 | | |
| | | | mg QOD if on 15 mg QD. No | | |
| | | | further dose ↑ until resolution | | |
| | | | of abnormality. | | |
| Platelets | <30,000 μL | None | Temporary interruption | Weekly repeat testing | At 15 mg QOD when |
| | | | | | platelets and other |
| | | | | | hematology parameters |
| | | | | | (neutrophils and |
| | | | | | hemoglobin) were at |
| | | | | | baseline level. |
| | >30% ↓ from | Platelets below | Reduce dose to previous dose | Weekly repeat testing | Not applicable. |
| | baseline | normal range at | level if up-titration has | | |
| | | baseline | occurred or reduce dose to 15 | | |
| | | | mg QOD if on 15 mg QD. No | | |
| | | | further dose ↑ until resolution | | |
| | | | of abnormality. | | |
| Neutrophils | <1.0 × 10³/μL | None | Temporary interruption | Weekly repeat testing | At 15 mg QOD when |
| | | | | | neutrophils and other |
| | | | | | hematology parameters |
| | | | | | (hemoglobin and platelets) |
| | | | | | were at baseline level. |
| | >30% ↓ from | Neutrophils below | Reduce dose to previous dose | Weekly repeat testing | Not applicable. |
| | baseline | normal range at | level if up-titration has | | |
| | | baseline | occurred or reduce dose to 15 | | |
| | | | mg QOD if on 15 mg QD. No | | |
| | | | further dose ↑ until resolution | | |
| | | | of abnormality. | | |
| Bilirubin | >2 × ULN | Accompanied by | Temporary interruption | Weekly repeat testing | At 15 mg QOD or less |
| | | ALT >3 × ULN, | | | frequent, when bilirubin is |
| | | indicative of liver | | | below ULN. Re-challenge |
| | | injury. | | | under these conditions |
| | | | | | requires approval of the |
| | | | | | Medical Monitor. |

| | | | | |
|---|---|---|---|---|
| UWDRS-III | Increase in UWDRS-III score from | Investigator and Medical | Report neurologic | Discuss with the Medical |
| and clinical | baseline as follows, AND is deemed | Monitor will evaluate the need | adverse event of special | Monitor. |
| neurological | clinically significant by the | for dose modification | interest and perform | |
| assessment | Investigator: | (interruption, increase or | UWDRS exam part I, II | |
| | Baseline UWDRS-III | decrease) based on copper | and III. Re-evaluate | |
| | score <20:≥4 point increase | control parameters and | neurologic status, | |
| | OR | relevant clinical data. | including complete | |
| | Baseline UWDRS-III | Rationale for dosing decision | UWDRS examination, no | |
| | score ≥20:≥6 point increase | must be documented in study | later than next study visit, | |
| | | record and re-evaluated at the | even if not planned per | |
| | | next study visit. | the SoA. | |
| Psychiatric | Clinically significant | Investigator and Medical | Re-evaluate psychiatric | Discuss with the Medical |
| assessment | signs of | Monitor will evaluate the need | status no later than next | Monitor. |
| | psychiatric worsening | for dose modification | study visit, even if not | |
| | | (interruption, increase or | planned per the SoA. | |
| | | decrease) based on copper | | |
| | | control parameters and | | |
| | | relevant clinical data. | | |
| | | Rationale for dosing decision | | |
| | | must be documented in study | | |
| | | record and re-evaluated at the | | |
| | | next study visit. | | |

^{a}For changes in safety monitoring, weekly repeat testing for laboratory parameters can be completed by a home healthcare nurse if a routine study visit is not scheduled during this time period.
^{b}A maximum of 3 re-challenges will be allowed for BC-TTM.
^{c}For re-challenges, patients who were on 15 mg QOD should be re-challenged at the 15 mg QOD dose.
^{d}The Investigator, in consultation with the Medical Monitor, may change dose and dose frequency in patients who require re-challenge.
Abbreviations: ALT = alanine aminotransferase; QD = once daily; QOD = every other day; SoA = Schedule of Activities; ULN = upper limit of normal; UWDRS = Unified Wilson Disease Rating Scale.

All patients were treated with TTM in the Extension Period. Patients who received TTM in the Primary Evaluation Period of Study 301 continued to receive the same dose they received at last study visit in the Primary Evaluation Period, individualized dosing was subsequently managed as described herein. Patients who transitioned from SoC in the Extension Period were administered TTM as described herein. Dose modification, if required, was made as described herein.

### Efficacy Assessments

The Week 48 visit was the end of the Primary Evaluation Period (PEP) and the beginning of the Extension Period (i.e., the Week 48 visit and the Extension Day 1 visit occurred on the same day). All assessments for the Week 48 visit were performed prior to dosing of TTM. Dosing of TTM on Extension Day 1 marked the beginning of the Extension Period. Patients who did not enter the Extension Period discontinued dosing at Week 48 and had a final study visit for safety follow up at Week 52.

Assessment of Copper: Measurements of plasma dNCC concentration were the primary assessment for the efficacy of TTM treatment in WD. The AUEC for plasma dNCC concentration over time aims to quantify the dynamic tissue Cu mobilization and Cu sequestration effect of TTM. This assessment is also applicable to SoC treatments. In addition, plasma Cp, CpC, plasma total copper, and LBC were measured with AUEC calculated for plasma total copper and LBC.

The LBC method measures exchangeable plasma copper that is not bound to either Cp or TPC.

Unified Wilson Disease Rating Scale (Parts I, II, and III): The UWDRS is a clinical rating scale designed to evaluate the neurological manifestations of WD that generally can be divided into 3 movement disorder syndromes: dystonic, ataxic, and Parkinsonian syndrome. The UWDRS comprises 3 parts: UWDRS Part I (level of consciousness, item 1), UWDRS Part II (a patient-reported review of daily activity items [disability], items 2 to 11), and UWDRS Part III (a detailed neurological examination, items 12 to 34).

The UWDRS Part I and Part III were assessed by a neurologist who is blinded to the treatment randomization, while UWDRS Part II may be reported to a non-blinded member of the study team by the patient, family member, or caregiver. The UWDRS has not been formally evaluated in adolescents. However, the components of Part I (level of consciousness), Part II (patient or caregiver-reported disability) and Part III (neurologic examination findings) were not fundamentally different between adults and adolescents. Patients aged 12 years and older were expected to be able to comply with UWDRS assessments.

Clinical Global Impression-Improvement Severity Scale and the Clinical Global Impression-Severity Improvement Scale: The Clinical Global Impression (CGI) rating scales were commonly used measures of symptom severity, treatment response, and the efficacy of treatments in treatment studies of adult and pediatric patients with mental disorders.

The Clinical Global Impression-Severity scale (CGI-S) is a 7-point scale that requires the clinician to rate the severity of the patient's illness at the time of assessment, relative to the clinician's past experience with patients who have the same diagnosis. Considering total clinical experience, a patient was assessed on severity of illness at the time of rating as: 1, normal, not at all ill; 2, borderline ill; 3, mildly ill; 4, moderately ill; 5, markedly ill; 6, severely ill; or 7, extremely ill.

The Clinical Global Impression-Improvement scale (CGI-I) is a 7-point scale that requires the clinician to assess how much the patient's illness has improved or worsened relative to a baseline state at the beginning of the intervention and rated as: 1, very much improved; 2, much improved; 3, minimally improved; 4, no change; 5, minimally worse; 6, much worse; or 7, very much worse.

### Adverse Events and Serious Adverse Events

An AE is any untoward medical occurrence in a participant or clinical investigation participant administered a pharmaceutical product and which does not necessarily have to have a causal relationship with this treatment (ICH E2A).

A SAE is defined as any untoward medical occurrence that, at any dose: results in death; is life-threatening; requires inpatient hospitalization or prolongation of existing hospitalization; results in persistent disability/incapacity; is a congenital anomaly/birth defect; or other situations that result in, for example, invasive or malignant cancers, intensive treatment in an emergency room or at home for allergic bronchospasm, blood dyscrasias or convulsions that do not result in hospitalization, or development of drug dependency or drug abuse.

Adverse events were reported by the patient (or, when appropriate, by a caregiver, surrogate, or the patient's legally authorized representative). The Investigator and any qualified designees were responsible for detecting, documenting, and recording events that meet the definition of an AE or SAE and remain responsible for following up AEs that were serious, considered related to the study intervention or study procedures, or that caused the patient to discontinue the study.

Adverse Events of Special Interest: Any new neurological symptom or clinically significant worsening of an ongoing neurological symptom after initiation of study drug (BC-TTM or SoC) was designated to be an AESI, whether serious or non-serious.

If a patient has an AESI, in addition to assessments deemed clinically relevant by the Investigator, the following assessments should be performed to the extent possible to help assess the AE and patient status: UWDRS Part III, non-verbal Stroop Interference Test, Digit Span Test, and the CGI-I and CGI-S. The Investigator or Sub Investigator can perform additional assessments or laboratory testing at their discretion.

### Pharmacokinetics, Pharmacodynamics, and Biomarkers

Pre-dose whole blood samples were collected at each designated visit for the measurement of the following TTM PK, PD, and biomarker analysis:

Pharmacokinetics: Blood samples for PK analysis were collected to measure plasma total molybdenum and plasma ultrafiltrate (PUF) molybdenum.

Pharmacodynamics: Blood samples were collected to directly measure plasma total copper, PUF copper, dNCC, and LBC. Calculations may be performed for cNCC and cNCC_{corrected}, the latter of which is non-ceruloplasmin-bound and non-molybdenum-albumin-bound copper.

Biomarkers and Biobank Samples: Blood samples were collected to measure plasma Cp and CpC. Urine samples were collected for analysis of urine copper and molybdenum. Additional biomarker or biobank samples were collected for the analysis of molybdenum and/or copper species associated with treatment.

### Statistical Considerations

A general description of the statistical methods used to analyze the efficacy and safety data is outlined below.

Statistical analyses were carried out using SAS®, Version 9.3 or later, SAS Institute, Cary, North Carolina, USA. Baseline for all assessments were defined as the last assessment yielding non-missing valid values taken prior to first dose of study drug (BC-TTM or SoC). Analysis Populations included:

The Full Analysis Set includes all randomized patients who received at least 1 dose of randomized treatment. Patients were analyzed as randomized.

The safety analysis was performed on the Safety Analysis Set. This dataset includes all patients who received at least 1 dose of randomized treatment. Patients were summarized according to the treatment actually received.

The Per-Protocol Set includes all patients who were randomized and had at least baseline and 48-week efficacy assessments for dNCC in the Primary Evaluation Period. Patients with major protocol deviations that were likely to impact the primary efficacy analysis were excluded from the Per-Protocol Set.

The Extension Analysis Set includes all patients who entered the Extension Period and received at least 1 dose of BC-TTM in the Extension Period.

### Primary Efficacy Analysis

The primary estimand is the difference in daily mean dNCC AUEC from 0 to 48 weeks between TTM and SoC in patients with WD, regardless of less-than-complete adherence or use of another medication that affects plasma dNCC, with no benefit derived from treatment after death.

The AUEC for dNCC concentration was calculated using the trapezoidal rule, and then divided by number of days to yield a mean daily AUEC plasma dNCC concentration from baseline to Week 48 (expressed as μmol/L and described as AUEC_{0-48 W}).

The AUEC_{0-48 W} was compared between TTM and SoC using an analysis of covariance (ANCOVA) statistical model; treatment arm, baseline plasma dNCC concentration, and cohort, will be included in the model. Tests were performed at a significance level of 0.05 (2-sided).

Model-based estimates of the difference between randomized treatments (TTM-SoC) in AUEC_{0-48 W}, along with a 2-sided 95% Cl and p-value were provided. If the lower 2-sided 95% Cl exceeds 0 μM, superiority was concluded.

Cohort 1: Patients previously treated for >28 days. The supportive analysis of the primary endpoint within Cohort 1 mirrored that described for the overall population analysis except that the analysis removed Cohort 2 from the model.

Cohort 2: Patients who were treatment-naïve or previously treated for ≤28 days. The AUEC_{0-48 W} was analyzed descriptively; there was no formal statistical comparison made between the randomized treatment arms. The AUEC_{0-48 W} was estimated using the same model terms as described for the analysis of Cohort 1 patients.

### Patient Population

Patients were eligible to be included in the study only if all of the following criteria applied:
1. Established diagnosis of WD by Leipzig-Score≥4 documented by testing as outlined in the 2012 European Association for the Study of Liver WD Clinical Practice Guidelines.
2.12 years of age or older at time of informed consent/assent (18 years or older in Germany).
3. Willing to withhold treatment with SoC for ≥48 hours immediately prior to first study assessment on Day 1.
4. Willing to avoid use of vitamins and/or minerals containing copper, zinc, or molybdenum throughout the study duration
5. Willing to avoid intake of foods and drinks with high contents of copper throughout the study duration.

Patients were excluded from the study if any of the following criteria applied:
1. Decompensated hepatic cirrhosis
2. MELD (Model for End-stage Liver Disease) score>13
3. Modified Nazer score>7
4. Clinically significant GI bleed within past 3 months
5. Alanine aminotransferase>2×upper limit of normal (ULN) for patients treated for >28 days with WD therapy (Cohort 1)
6. Alanine aminotransferase>5×ULN for treatment-naïve patients or patients who have been treated for s 28 days (Cohort 2)
7. Marked neurological disease requiring either nasogastric feeding or intensive inpatient medical care
8. Hemoglobin<9 g/dL
9. Participation in a clinical study of an experimental or unapproved/unlicensed therapy during the screening period or within 4 weeks prior to informed consent
10. History of seizure activity within 6 months prior to informed consent
11. Pregnant (or women who were planning to become pregnant) or breastfeeding women
12. Known sensitivity to BC-TTM, BC-TTM excipients (anhydrous di-calcium phosphate, anhydrous sodium carbonate), or any of the ingredients contained in BC-TTM or related compounds
13. Active infection with hepatitis B virus (positive hepatitis B surface antigen) or C virus (patients with positive hepatitis C antibody result would require confirmation of active disease with a positive hepatitis C polymerase chain reaction test), or seropositivity for human immunodeficiency virus (HIV)
14. Previous treatment with tetrathiomolybdate
15. Any disease, disability, illness or abnormal laboratory values in the opinion of the Investigator, compromise patient safety or interfere with the collection or interpretation of study results
16. Patients with end-stage renal disease on dialysis (chronic kidney disease stage 5 [CKD 5]) or creatinine clearance<30 mL/min.

Patients in the Primary Evaluation Period were randomized to either TTM or SoC treatment, and enrolled into 1 of 2 cohorts as noted above. All patients in the Extension Period will receive treatment with TTM.

Approximately 180 eligible patients with WD aged 12 years and older (18 years and older in Germany) were enrolled in this study. The assignment per Cohort and treatment is provided in Table 2.

TABLE 2
Patient assignment per Cohort and Treatment

2:1 Ratio

| | | TTM | SoC | Total |
|---|---|---|---|---|
| 3:1 Ratio | Cohort 1 | 90 | 45 | 135 |
| | Cohort 2 | 30 | 15 | 45 |
| | Total | 120 | 60 | 180 |

Ultimately 214 patients enrolled; all had preserved liver function and 79% had neurological symptoms. Of the 214 patients, 207 were randomized: 137 to TTM (with 104 to Cohort 1 and 33 to Cohort 2) and 70 to SoC (with 56 to Cohort 1 and 14 to Cohort 2). Overall patient demographics are provided in Tables 3-1 and 3-2, and the history of prior treatment for WD is provided in Table 4.

TABLE 3-1
Patient Demographics

| | | TTM | | SoC | |
|---|---|---|---|---|---|
| Variable | Statistic | N = 137 | | N = 70 | |
| Sex | % Male | 59.9 | | 52.9 | |
| Race | % White | 78.1 | | 75.7 | |
| BMI^{§} (kg/m²) | Mean (SD) | 23.9 | (4.75) | 23.8 | (4.27) |
| Age (years) | Mean (SD) | 34.3 | (13.67) | 32.1 | (13.37) |
| >=12 to <18 years | N (%) | 12 | (8.8) | 6 | (8.6) |
| >=18 to <65 years | N (%) | 123 | (89.8) | 62 | (88.6) |
| >=65 years | N (%) | 2 | (1.5) | 2 | (2.9) |

^{§}<18.5 is underweight, ≥18.5 to <25 is normal, ≥25.0 to <30 is overweight, ≥30.0 is obese

TABLE 3-2
Patient and Baseline Characteristics

| | | Cohort 1 | | | | Cohort 2 | | | |
|---|---|---|---|---|---|---|---|---|---|
| | | TTM | | SoC | | TTM | | SoC | |
| Variable | Statistic | N = 104 | | N = 56 | | N = 33 | | N = 14 | |
| Sex | % Male | 58 | (55.8) | 26 | (46.4) | 24 | (72.7) | 11 | (78.6) |
| Race | % White | 80 | (76.9) | 41 | (73.2) | 27 | (81.8) | 12 | (85.7) |
| BMI^{§} (kg/m²) | Mean (SD) | 23.7 | (4.79) | 23.6 | (4.29) | 24.7 | (4.58) | 24.8 | (4.23) |
| Age (years) | Mean (SD) | 36.2 | (14.11) | 33.7 | (14.17) | 28.5 | (10.34) | 26.0 | (7.01) |
| ≥12 to <18 years | N (%) | 10 | (9.6) | 4 | (7.1) | 2 | (6.1) | 2 | (14.3) |
| ≥18 to <65 years | N (%) | 92 | (88.5) | 50 | (89.3) | 31 | (93.9) | 12 | (85.7) |
| ≥65 years | N (%) | 2 | (1.9) | 2 | (3.6) | 0 | | 0 | |
| dNCC | Mean (SD) | 0.799 | (0.6432) | 0.631 | (0.4198) | 1.885 | (1.0432) | 2.300 | (2.1408) |
| UWDRS Part II score (0 to 40) | Mean (SD) | 4.0 | (8.0) | 3.5 | (6.5) | 3.7 | (6.6) | 3.6 | (6.9) |
| UWDRS Part III score (0 to 175) | Mean (SD) | 16.0 | (20.7) | 12.8 | (18.4) | 15.1 | (19.4) | 6.5 | (9.8) |
| CGI-S score (1 to 7) | Mean (SD) | 2.9 | (1.6) | 2.4 | (1.3) | 3.1 | (1.7) | 2.7 | (1.7) |
| MELD score (6 to 40) | Mean (SD) | 8.4 | (1.8) | 8.4 | (2.2) | 8.9 | (1.8) | 8.0 | (1.9) |

| Cirrhosis | % | 34.6 | 46.4 | 30.3 | 28.6 |
|---|---|---|---|---|---|
| Race | | | | | |
| Asian | % | 18.3 | 23.2 | 15.2 | 0 |
| Black or African American | % | 1 | 3.6 | 0 | 0 |
| White | % | 76.9 | 73.2 | 81.8 | 85.7 |
| Other | % | 2.9 | 0 | 0 | 14.3 |
| Unknown | % | 0 | 0 | 3.0 | 0 |
| Not Reported | % | 1.0 | 1.0 | 1.0 | 1.0 |

^{§}<18.5 is underweight, ≥18.5 to <25 is normal, ≥25.0 to <30 is overweight, ≥30.0 is obese.
At the neurological examination (UWDRS Part III), signs/symptoms were present in 79% of patients but scores were mild for most. Mean CGI-S scores indicate that patients were mildly ill. Compensated cirrhosis was reported for 28.6-46.4% of patients at baseline, and no patients with decompensated cirrhosis were included.

TABLE 4
Prior Treatments for WD

| | Cohort 1 | | Cohort 2 | |
|---|---|---|---|---|
| | TTM | SoC | TTM | SoC |
| Variable | N = 104 | N = 56 | N = 33 | N = 14 |

Cumulative Duration of Prior WD Treatment (months)

| N | 104 | | 56 | | 17 | | 5 | |
|---|---|---|---|---|---|---|---|---|
| Mean (SD) | 155.5628 | (147.31796) | 144.7897 | (149.95991) | 0.6068 | (0.24589) | 0.6308 | (0.31718) |
| Median (IQR) | 116.1561 | (185.9548) | 100.4517 | (193.4456) | 0.6571 | (0.3943) | 0.7556 | (0.2300) |
| Q1, Q3 | 41.6, | 228 | 26.9, | 220 | 0.427, | 0.821 | 0.591, | 0.821 |
| Min, Max | 2.201, | 672.526 | 1.248, | 624.789 | 0.066, | 0.920 | 0.099, | 0.887 |

Cumulative History of Prior WD Therapy (N (%)

| | | | | | | | | |
|---|---|---|---|---|---|---|---|---|
| Zinc Monotherapy | 15 | (14.4) | 9 | (16.1) | 7 | (41.2) | 1 | (20.0) |
| Penicillamine or Trientine (±Zinc) | 89 | (85.6) | 47 | (83.9) | 10 | (58.8) | 4 | (80.0) |
| Penicillamine (±Zinc) | 40 | (38.5) | 25 | (44.6) | 6 | (35.3) | 4 | (80.0) |

| | | | | | | | |
|---|---|---|---|---|---|---|---|
| Trientine (±Zinc) | 22 | (21.2) | 10 | (17.9) | 4 | (23.5) | 0 |

| | | | | | | |
|---|---|---|---|---|---|---|
| Penicillamine + Trientine (±Zinc) | 27 | (26.0) | 12 | (21.4) | 0 | 0 |

Ultimately 184 patients completed the 48-week Treatment Period: 119 to TTM (with 91 to Cohort 1 and 28 to Cohort 2) and 65 to SoC (with 52 to Cohort 1 and 13 to Cohort 2); and 178 patients entered the Extension Period: 117 to TTM (with 89 to Cohort 1 and 28 to Cohort 2) and 61 to SoC (with 49 to Cohort 1 and 12 to Cohort 2).

The number of patients in the SoC group who received each treatment was as follows: Zinc monotherapy: Cohort 1, n=23 (41%), Cohort 2, n=0 (0%), Overall, n=23 (33%); Penicillamine (±zinc): Cohort 1, n=19 (39%), Cohort 2, n=10 (71%), Overall, n=29 (41%); Trientine (±zinc): Cohort 1, n=14 (25%), Cohort 2, n=4 (29%), Overall, n=18 (26%).

### Copper Mobilization and Sequestration

The primary objective of the study was to evaluate the efficacy of TTM administered for 48 weeks, compared to standard of care (SoC), on copper control in WD patients aged 12 years and older (or 18 year and older in Germany). The results from the primary endpoint, daily mean AUEC_{0-48 W} of dNCC, for the primary evaluation period are provided in Table 4. These results show superiority of TTM over SoC in mobilizing copper as demonstrated by the primary efficacy endpoint, daily mean dNCC AUEC_{0-48 wks}; the value for TTM is 3.18 (standard error [SE]=0.167) and for SoC is 1.00 (SE=0.219), with a p<0.0001 for the difference.

TABLE 5
Mean Daily dNCC AUEC_{0-48 w} (μmol/L) with multiple imputation; Full Analysis Set

| | | Cohort 1 | | | | Cohort 2 | | | | Overall | | | |
|---|---|---|---|---|---|---|---|---|---|---|---|---|---|
| | | TTM | | SoC | | TTM | | SoC | | TTM | | SoC | |
| Statisticⁱ | | N = 104 | | N = 56 | | N = 33 | | N = 14 | | N = 137 | | N = 70 | |
| n | | 91 | | 51 | | 27 | | 12 | | 118 | | 63 | |
| Mean (SD) | | 2.68 | (2.118) | 0.72 | (0.643) | 4.58 | (2.526) | 1.09 | (0.484) | 3.12 | (2.347) | 0.79 | (0.629) |
| LSMⁱⁱ (SE) | | 2.50 | (0.150) | 0.87 | (0.204) | 4.76 | (0.319) | 0.96 | (0.487) | 3.18 | (0.167) | 1.00 | (0.219) |
| 95% CI | | (2.21, | 2.79) | (0.46, | 1.27) | (4.13, | 5.38) | (0.01, | 1.92) | (2.85, | 3.51) | (0.58, | 1.43) |
| LSM Diff (SE) | | 1.64 | (0.254) | | | 3.79 | (0.584) | | | 2.18 | (0.244) | | |
| 95% CI | | (1.14, | 2.13) | | | (2.65, | 4.94) | | | (1.70, | 2.65) | | |
| P-value | | <0.0001 | | | | <0.0001 | | | | <0.0001 | | | |
| Within | LSM (SE) | 2.59 | (0.165) | 0.88 | (0.221) | 4.64 | (0.315) | 0.95 | (0.472) | 3.06 | (0.179) | 0.93 | (0.226) |
| Group | 95% CI | (2.27, | 2.92) | (0.44, | 1.32) | (4.00, | 5.28) | (0.00, | 1.91) | (2.70, | 3.41) | (0.49, | 1.38) |
| Between | LSM Diff | 1.72 | (0.277) | | | 3.68 | (0.568) | | | 2.12 | (0.253) | | |
| Group | (SE) | | | | | | | | | | | | |

| | | | |
|---|---|---|---|
| P-value | <0.0001 | <0.0001 | <0.0001 |

ⁱData analyzed using an ANOCA model that included treatment, cohort, and baseline value. For cohort analysis, analysis was performed on each cohort using ANCOVA model, cohort term was removed from model. Analysis results were combined using Rubin's rules. Missing imputation was performed: 1) for intermediate missing, interpolation was used to fill out missing values. 2) For patients who died, baseline dNCC was carried forward from discontinuation to week 48. 3) For others, multiple imputation was used to impute missing dNCC values assuming data were missing not at random. Responses for both treatment groups were imputed with multiple imputation based on the response for SoC patients. Cohort, baseline, and previous visit value were included in the imputation model.
ⁱⁱLeast Squares Means (LSM) is a statistical method that determines the line of best fit for a dataset.

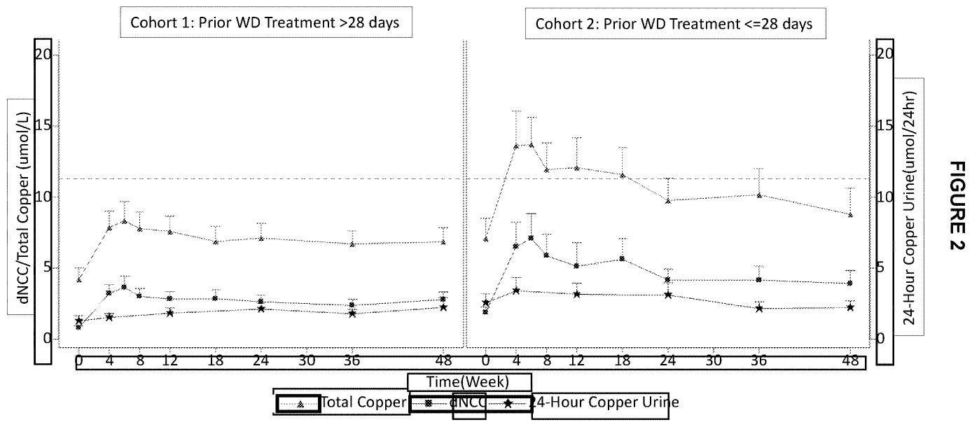

The study results show that TTM is statistically superior to SoC for mobilizing copper from tissue (FIG. 2). Overall, mean daily dNCC AUEC_{0-48 W} (μmol) was 3.2-fold higher in patients treated with TTM versus SoC. Overall results, and those in each cohort, were statistically significant (P<0.00001). Mean values for patients treated with TTM were 2.9-fold higher in treatment experienced (Cohort 1) and 4.9-folder higher in treatment inexperienced patients (Cohort 2). While more copper was mobilized in treatment inexperienced patients (Cohort 2) than in treatment experienced (Cohort 1), these results show that tissue-bound copper remains built-up in the tissue even in patients who have been on SoC therapy for many years, e.g., an average of ten or more years. As can be seen from the results in Table 4, duration of prior treatment does not meaningfully affect the magnitude of benefit even though the magnitude of effect differs between the cohorts. In contrast to SoC, BC-TMM has significant impact on mobilizing and safely sequestering copper from tissues.

TTM mobilized copper even in Cohort 1 participants who had been on SoC therapy for a mean of more than 10 years. As shown in Table 5, the daily mean dNCC AUEC_{0-48 weeks} of SoC assessed at equivalent time points to TTM is measurable but low (1.0 or less), regardless of prior treatment status.

The plot of plasma dNCC over time in participants treated with TTM shows an immediate rise, peak at 4 to 6 weeks, and gradual return toward baseline by 48 weeks (FIG. 2). Tissue copper mobilization is seen in both cohorts, even in Cohort 1 participants who had been treated with SoC for an average of 10 years. Further, FIG. 2 also shows that the time until dNCC returns to baseline is greater (longer) for Cohort 2 compared with Cohort 1; this reflects greater mobilization of larger excess tissue copper stores among participants with little or no prior treatment.

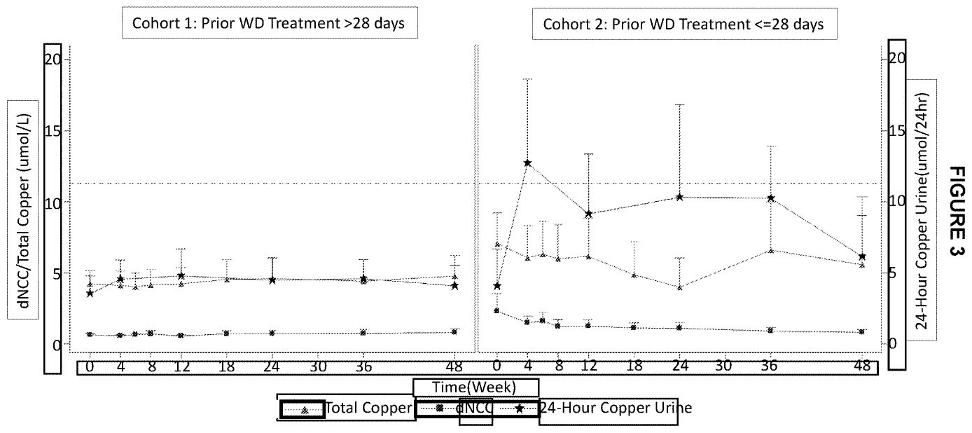

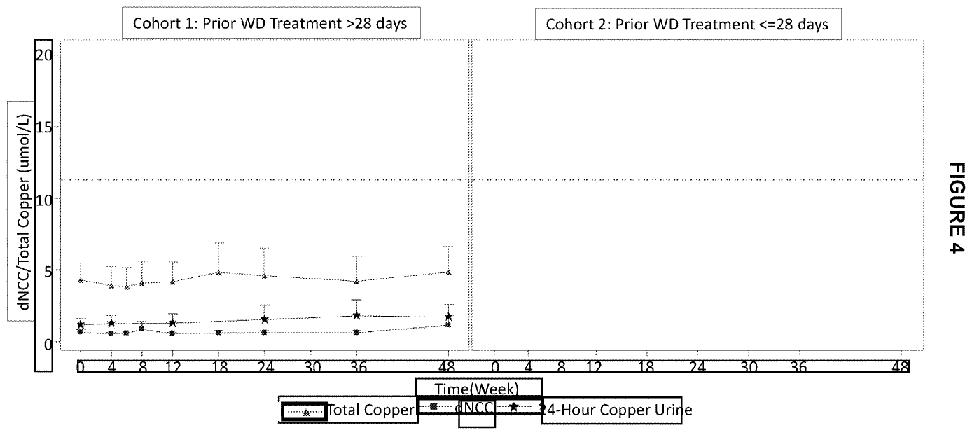

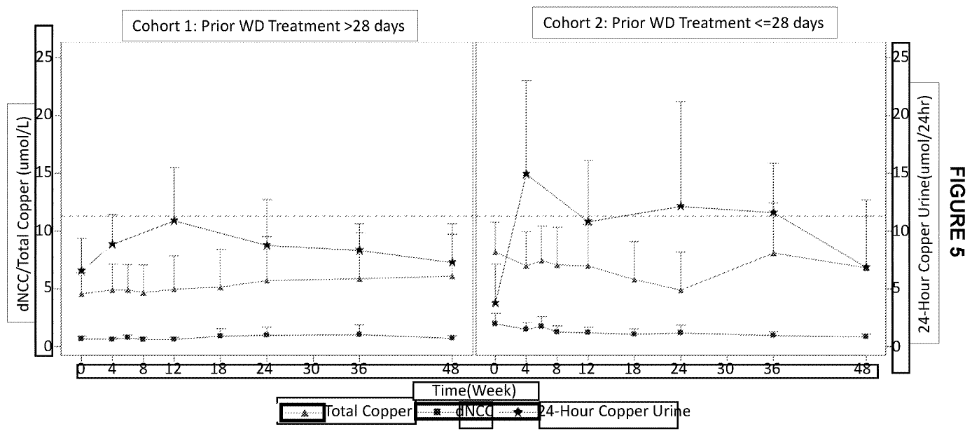

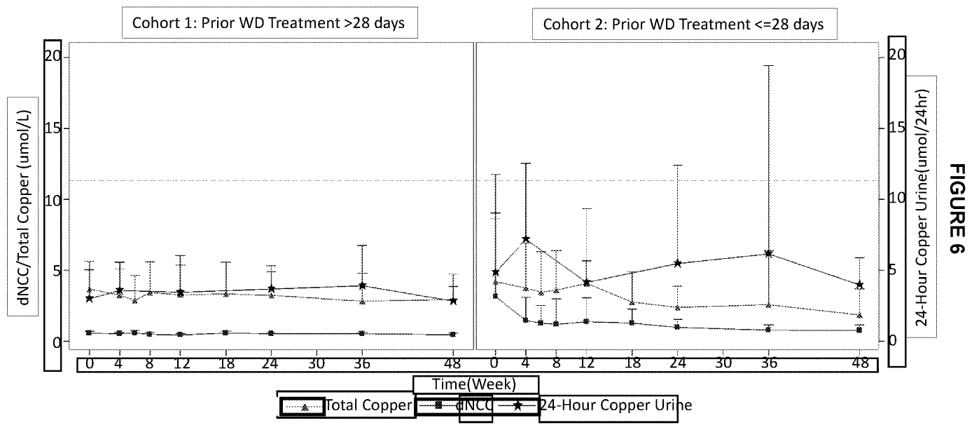

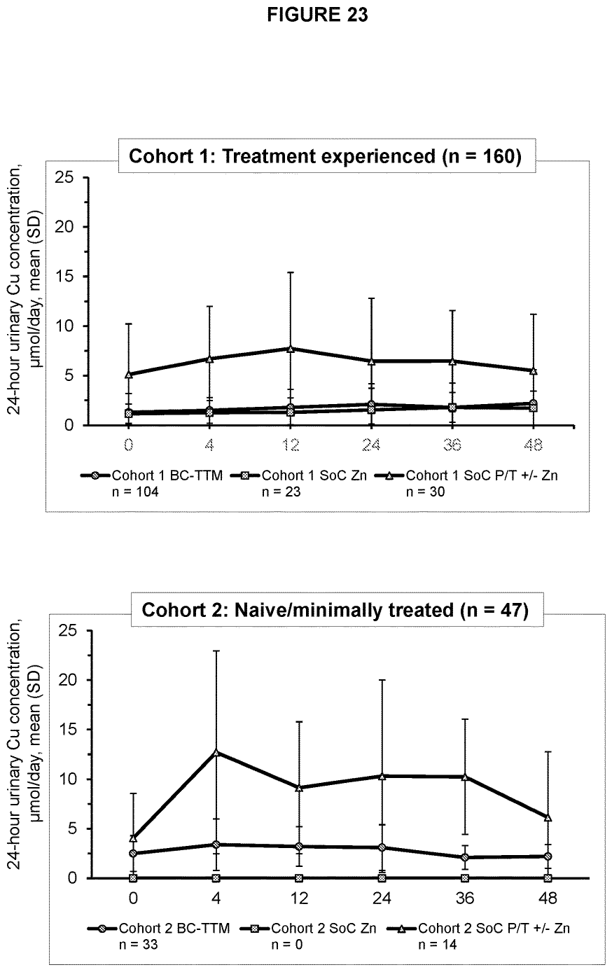

For participants treated with SoC, the plots of plasma dNCC at equivalent time points (where available) were essentially flat (FIG. 3, FIG. 4, FIG. 5, and FIG. 6), indicating minimal change in the circulating non-ceruloplasmin bound copper. FIG. 23 shows that 24-hour urinary copper was lower with TTM than SoC chelator therapy. Overall, there was little change in the levels of either plasma total copper or of non-ceruloplasmin bound copper during SoC treatment. The superiority of TTM over SoC in mobilizing copper from tissues has been demonstrated.

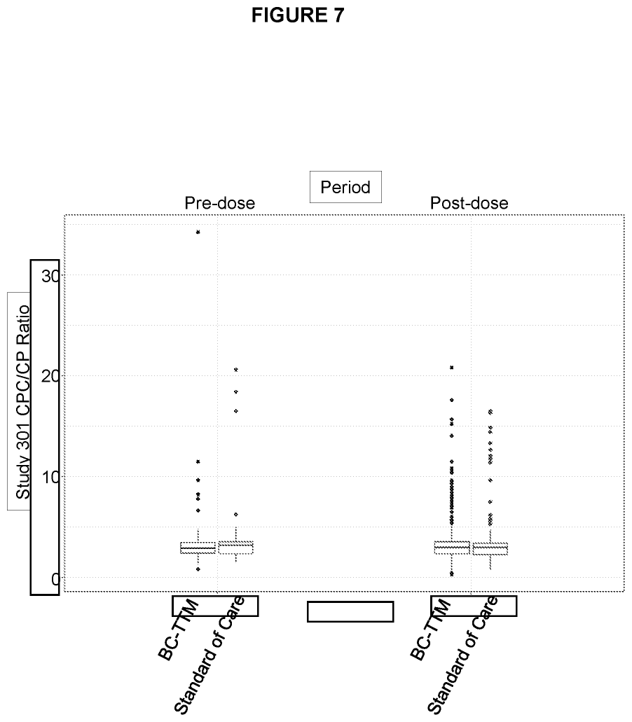

To evaluate whether tissue copper mobilization from TTM treatment have any effect on the most important plasma copper carrier ceruloplasmin (Cp) and the number of copper atoms carried per molecule of ceruloplasmin (CpC), Table 6 summarizes plasma CpC/Cp ratios. An average of 3 to 4 copper molecules were bound per molecule of ceruloplasmin at pre-dose baseline. Data were also presented in FIG. 7 for visual inspection. The average CpC/Cp values post-dose during the entire 48-week treatment period remained relatively stable for TTM while slightly reduced for SoC. This implies that both TTM and SoC have limited impact on the CpC/Cp ratio over 48 weeks of treatment. This observation is likely due to either the much higher binding affinity of ceruloplasmin to copper or limited, if any, TTM and SoC impact on holoceruloplasmin formation by the liver.

TABLE 6
Summary of Plasma CpC/Cp Ratio by Treatment (TTM vs Standard
of Care) in Study 301 (PD and Biomarker Analysis Set)

| | CpC/Cp Ratio | | | |
|---|---|---|---|---|
| | Treatment | | | |
| | TTM | TTM | SoC | SoC |
| | Period | | | |
| | | Post-dose | | Post-dose |
| | Pre-dose | to Week 48 | Pre-dose | to Week 48 |
| N/n | N = 137 | n = 1010 | N = 70 | n = 505 |
| Mean | 3.312 | 3.122 | 3.619 | 3.062 |
| SD | 2.987 | 1.563 | 3.290 | 1.686 |
| CV % | 1.109 | 1.998 | 1.100 | 1.816 |
| Median | 2.890 | 2.963 | 3.127 | 2.946 |
| Minimum | 0.806 | 0.297 | 1.377 | 0.695 |
| Maximum | 34.240 | 20.771 | 20.567 | 16.477 |

Preliminary population PK analysis including data from healthy participants (Studies 104, 106, 107, 108, and 109) and those with WD (Studies 201 and 301) has shown that age is not a significant covariate for plasma total molybdenum clearance. Elimination half-life was also similar across the age subgroups. Study 106 is a phase 1 study that assessed the pharmacokinetics (PK), pharmacodynamics (PD), biomarkers, and safety of TTM in healthy Japanese and non-Japanese subjects, and is described in Example 1 of U.S. Provisional Patent Application No. 63/339,307, filed May 6, 2022, and incorporated by reference herein. Study 104 is registered with EudraCT under study number 2019-000516-28; Study 107 is registered with ClinicalTrials.gov, number NCT04560816; Study 108 is registered with ClinicalTrials.gov, number NCT04594252; and Study 109 is registered with ClinicalTrials.gov, number NCT04610580.

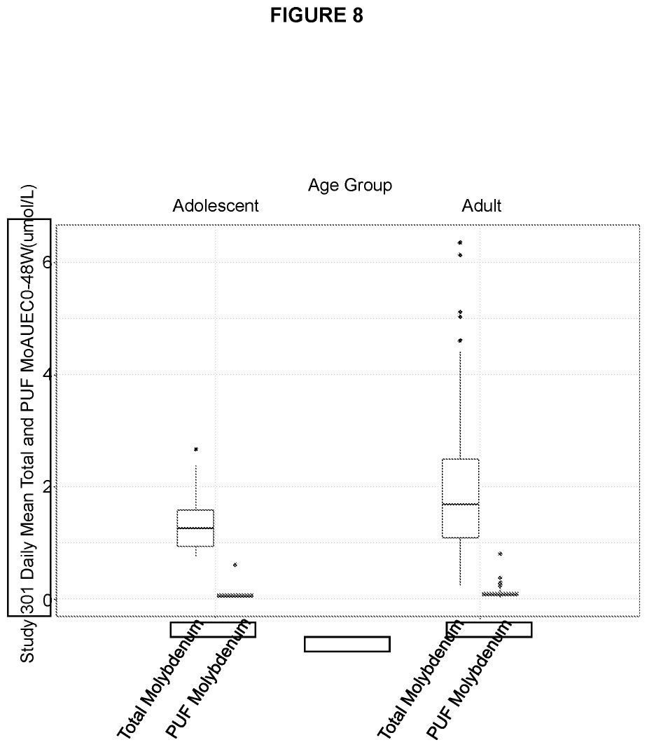

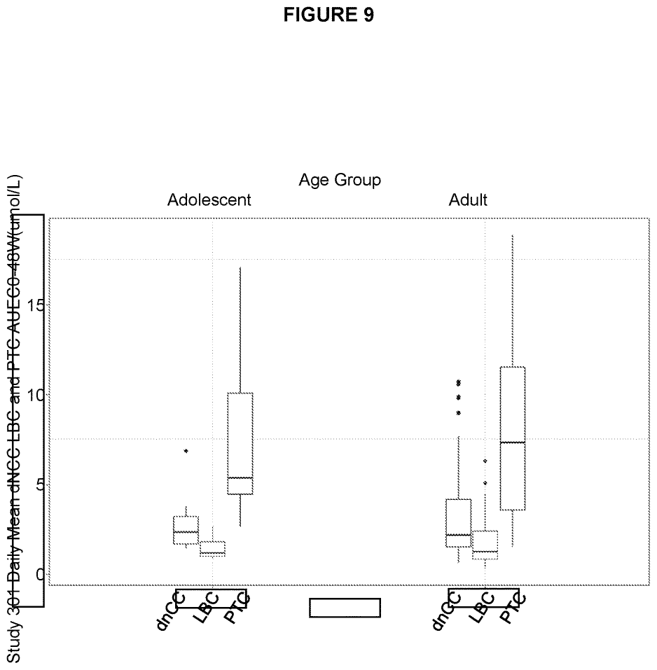

After 48 weeks of treatment with TTM in Study 301, the calculated plasma total molybdenum AUC_{(0-48 weeks)} (Table 7 and FIG. 8) and values for plasma total copper, dNCC, and LBC AUEC_{( )} (Table 8 and FIG. 9) have shown comparable central values for adults and adolescents.

TABLE 7
Summary of Calculated AUC_{(0-48 weeks)} Values for Plasma
Total and Ultrafiltrate Molybdenum by Age Group (Adult
vs Adolescent; Study 301) (PK Analysis Set)

| | Daily Mean AUC_{(0-48 weeks}) (mmol/L) | | | |
|---|---|---|---|---|
| | Matrix | | | |
| | Total Mo | Total Mo | PUF Mo | PUF Mo |
| | Age Group | | | |
| | Adolescent | Adult | Adolescent | Adult |
| N* | 12 | 106 | 10 | 105 |
| Mean | 1.4 | 1.9 | 0.1 | 0.1 |
| SD | 0.6 | 1.2 | 0.2 | 0.09 |
| CV % | 44.4 | 61.3 | 151.6 | 80.5 |
| Median | 1.3 | 1.7 | 0.05 | 0.09 |
| Minimum | 0.7 | 0.3 | 0.04 | 0.02 |
| Maximum | 2.7 | 6.4 | 0.6 | 0.8 |

TABLE 8
Summary of Calculated AUEC_{(0-48 weeks)} Values for Plasma
Total Copper, dNCC, and LBC by Age Group (Adult vs Adolescent;
Study 301) (PD and Biomarker Analysis Set)

| AUEC_{(0-48 week)} | | | | | | |
|---|---|---|---|---|---|---|
| (mmol/L) | dNCC | dNCC | LBC | LBC | PTC | PTC |
| Age Group | Adolescent | Adult | Adolescent | Adult | Adolescent | Adult |
| N* | 12 | 106 | 12 | 106 | 12 | 108 |
| Mean | 2.7 | 3.2 | 1.5 | 1.8 | 7.5 | 7.8 |
| SD | 1.5 | 2.4 | 0.7 | 1.3 | 4.7 | 4.7 |
| CV % | 56.2 | 76.7 | 44.4 | 69.8 | 63.1 | 59.5 |
| Median | 2.3 | 2.2 | 1.2 | 1.2 | 5.4 | 7.3 |
| Minimum | 1.5 | 0.6 | 0.8 | 0.3 | 2.6 | 1.6 |
| Maximum | 6.9 | 10.7 | 2.7 | 6.3 | 17.1 | 18.9 |

Study 204 is an exploratory study with aim to investigate the effects of TTM on Cu balance in participants with WD. Study 204 is registered with ClinicalTrials.gov, number NCT04573309, and is described in Example 1 of U.S. Provisional Patent Application No. 63/237,120, filed Aug. 25, 2021, and incorporated by reference herein. Study 204 specifically evaluates the effects of the 15 mg and 30 mg doses BC-TTM as well as the duration of treatment on Cu balance. The participants remained on a Cu-controlled diet, and Cu and Mo balance were measured on all intake (i.e., investigational agent, food and fluids) and all output (urine and feces). The Cu and Mo concentration of each sample was determined by inductively coupled plasma mass spectrometry (ICP-MS). Copper and Mo content of all intake and output was calculated based on the volume or weight of intake and output and the concentration of representative samples.

Collection periods for feces and urine varied in duration from 3 to 15 days to support assessment of both Cu and Mo balance before and at steady state for both 15 mg and 30 mg. Equilibration periods on Cu/Mo-controlled diets were a minimum of 48 hours. Copper balance was calculated as the mean daily Cu balance over each of the 4 collection periods. The interpretation of Cu balance was based on the criteria previously established when undertaking Cu balance studies with zinc treatment. For assessment of TTM effect on Cu balance, the time period for analysis took into consideration the average bowel transit of approximately 40 hours (male: 33 hours; female: 47 hours).

The interim results of Study 204 showed a net increase in daily fecal copper output after exposure of participants with WD to TTM. Results are available for the initial 3 participants enrolled in this open-label copper balance/molybdenum mass balance study. These participants had all intake (food and drink) and output (urine and feces) collected from Day −4 through Day −1 (baseline), from Day 1 through Day 8, and Day 25 through Day 39. They were to be dosed with BC-TTM at 15 mg/day for 28 days (Period 1), then at 30 mg/day for 11 days (Period 2); however, only 1 participant was able to escalate to the higher dose while the other 2 reduced the dose to 15 mg QOD due to alanine aminotransferase (ALT) elevations.

The patient information of Study 204 is provided in Table 9. The results from the primary endpoint of Study 204, mean daily Cu balance, are provided in Table 10. As noted above, mean daily Cu balance is measured by the calculated difference between Cu intake (in food and drink) and Cu output (in feces and urine) during TTM accumulation and steady-state periods for each dose.

TABLE 9
Study 204 Patient Demographics, Baseline Characteristics
and Prior Treatments for WD

| | Statistic/ | | |
|---|---|---|---|
| Variable | Category | Value | |
| Age (in years) | Mean (SD) | 31.7 | (7.23) |
| Sex | Female, n (%) | 1 | (33.3) |
| Race | White, n (%) | 3 | (100) |
| BMI^{§} (kg/m²) | Mean (SD) | 31.20 | (7.363) |
| Time Since WD Diagnosis (months) | Mean (SD) | 89.21 | (69.802) |
| Cirrhosis | Yes, n (%) | 1 | (33.3) |
| Cumulative Duration of Prior WD | Mean (SD) | 76.9 | (81.59) |
| Treatment (months) | | | |
| Prior WD Therapy: | | | |
| Zinc Monotherapy | | 1 | (33.3) |
| Penicillamine or Trientine (±Zinc) | | 2 | (66.7) |
| Penicillamine (±Zinc) | | 1 | (33.3) |
| Penicillamine + Trientine (±Zinc) | | 1 | (33.3) |

^{§}<18.5 is underweight, ≥18.5 to <25 is normal, ≥25.0 to <30 is overweight, ≥30.0 is obese

TABLE 10
Daily Net Copper Balance

| | Total (N = 3) | | | |
|---|---|---|---|---|
| Daily Net Copper Balance (mg) | Value | | Change from Baseline | |
| Baseline | | | | |
| N | 3 | | | |
| Mean (SD) | 1.2503 | (0.33263) | | |
| Median (IQR) | 1.2893 | (0.6618) | | |
| Q1, Q3 | 0.8999, | 1.5617 | | |
| Min, Max | 0.900, | 1.562 | | |
| 95% CI | (0.4240, | 2.0766) | | |
| Days 1-8 (Accumulation 15 mg/day) | | | | |
| N | 3 | | 3 | |
| Mean (SD) | 0.7654 | (0.35380) | −0.4848 | (0.11902) |
| Median (IQR) | 0.6678 | (0.6871) | −0.4291 | (0.2176) |
| Q1, Q3 | 0.4707, | 1.1578 | −0.6215, | −0.4039 |
| Min, Max | 0.471, | 1.158 | −0.621, | −0.404 |
| 95% CI | (−0.1135, | 1.6443) | (−0.7805, | −0.1892) |
| Days 25-28 (Steady State 15 mg/day) | | | | |
| N | 3 | | 3 | |
| Mean (SD) | 0.7638 | (0.91381) | −0.4865 | (0.59933) |
| Median (IQR) | 1.1434 | (1.7052) | −0.1459 | (1.0434) |
| Q1, Q3 | −0.2787, | 1.4266 | −1.1785, | −0.1351 |
| Min, Max | −0.279, | 1.427 | −1.179, | −0.135 |
| 95% CI | (−1.5063, | 3.0338) | (−1.9753, | 1.0023) |
| Days 31-35 (Accumulation 30 mg/day) | | | | |
| N | 3 | | 3 | |
| Mean (SD) | 0.8577 | (0.34265) | −0.3925 | (0.05781) |
| Median (IQR) | 0.8312 | (0.6838) | −0.3707 | (0.1093) |
| Q1, Q3 | 0.5291, | 1.2129 | −0.4581, | −0.3488 |
| Min, Max | 0.529, | 1.213 | −0.458, | −0.349 |
| 95% CI | (0.0065, | 1.7089) | (−0.5362, | −0.2489) |
| Days 36-39 (Steady State 30 mg/day) | | | | |
| N | 3 | | 3 | |
| Mean (SD) | 0.8582 | (0.25646) | −0.3921 | (0.11676) |
| Median (IQR) | 0.7986 | (0.5024) | −0.4225 | (0.2275) |
| Q1, Q3 | 0.6367, | 1.1391 | −0.4906, | −0.2632 |
| Min, Max | 0.637, | 1.139 | −0.491, | −0.263 |
| 95% CI | (0.2211, | 1.4952) | (−0.6822, | −0.1021) |

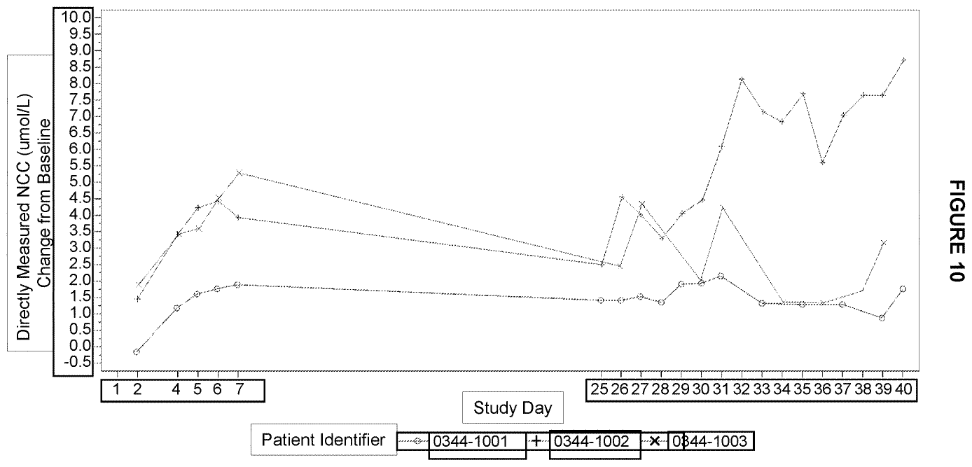

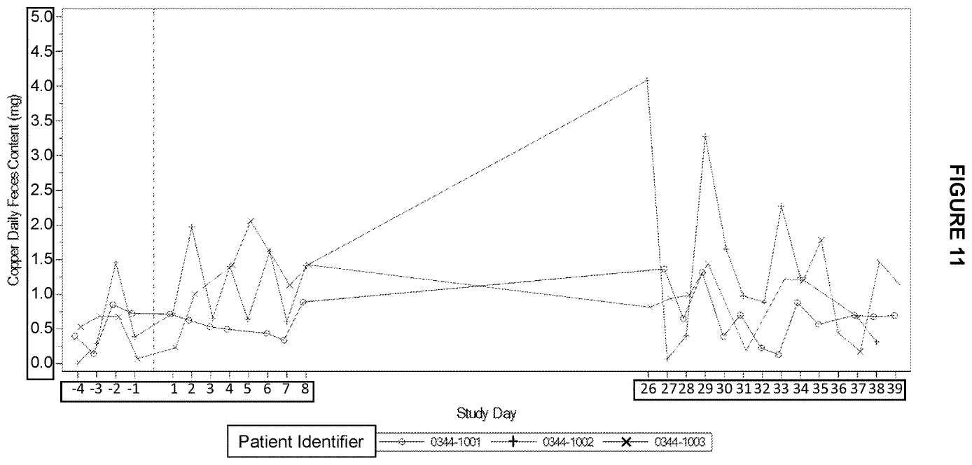

Based on interim results from the 3 participants with WD in Study 204, copper mobilization consequent to treatment with 15 mg/day (or every other day) or 30 mg/day for a total duration of 39 days was significant compared with pre-dose baseline. Consistent results among the 3 participants completing the study to date indicate that TTM produces rapid and sustained mobilization of copper from tissues into blood where copper is safely sequestered in the tripartite complex. This is evidenced by the rapid and sustained increase from baseline in dNCC displayed in Table 11 and FIG. 10, and the increase from baseline in fecal copper excretion displayed in Table 12 and FIG. 11.

TABLE 11
TTM Mobilization of Copper from Tissue to Blood: Increase from
Baseline in Directly Measured NCC (μmol/L) (Full Analysis Set)

| Study Treatment Day | Time | N | Mean (SD) | | 95% CI | |
|---|---|---|---|---|---|---|
| Day 1 | 2 Hours Post-Dose | 3 | 0.50042 | (0.710475) | (−1.26449, | 2.26534) |
| | 4 Hours Post-Dose | 3 | 1.96026 | (1.803672) | (−2.52031, | 6.44083) |
| | 5 Hours Post-Dose | 3 | 2.71929 | (2.533892) | (−3.57525, | 9.01383) |
| | 6 Hours Post-Dose | 3 | 2.32430 | (2.086336) | (−2.85844, | 7.50705) |
| | 8 Hours Post-Dose | 3 | 2.40193 | (2.182723) | (−3.02025, | 7.82412) |
| | 12 Hours Post-Dose | 3 | 1.72473 | (1.575050) | (−2.18791, | 5.63738) |
| | 24 Hours Post-Dose | 3 | 1.06327 | (1.074232) | (−1.60527, | 3.73181) |
| Day 4 | Pre-Dose | 3 | 2.67733 | (1.296508) | (−0.54338, | 5.89803) |
| Day 5 | Pre-Dose | 3 | 3.14418 | (1.369506) | (−0.25786, | 6.54622) |
| Day 6 | Pre-Dose | 3 | 3.57431 | (1.569784) | (−0.32525, | 7.47387) |
| Day 7 | Pre-Dose | 3 | 3.70021 | (1.708767) | (−0.54460, | 7.94502) |
| Day 25 | −0.5 Hours | 3 | 2.12130 | (0.611005) | (0.60348, | 3.63912)* |
| | 2 Hours Post-Dose | 3 | 2.55143 | (1.063471) | (−0.09038, | 5.19324) |
| | 4 Hours Post-Dose | 3 | 3.44318 | (2.008214) | (−1.54551, | 8.43186) |
| | 5 Hours Post-Dose | 3 | 3.38023 | (2.477964) | (−2.77538, | 9.53583) |
| | 6 Hours Post-Dose | 3 | 4.43983 | (2.949162) | (−2.88630, | 11.76595) |
| | 8 Hours Post-Dose | 3 | 5.10077 | (2.491247) | (−1.08783, | 11.28937) |
| | 12 Hours Post-Dose | 3 | 4.91717 | (2.463080) | (−1.20146, | 11.03580) |
| | 24 Hours Post-Dose | 3 | 3.43793 | (1.754247) | (−0.91986, | 7.79572) |
| Day 27 | Pre-Dose | 2 | 2.76729 | (1.754803) | NA | |
| Day 28 | Pre-Dose | 2 | 2.32666 | (1.376469) | NA | |
| Day 29 | −0.5 Hours | 3 | 2.66159 | (1.208535) | (−0.34058, | 5.66376) |
| | 2 Hours Post-Dose | 3 | 2.81371 | (0.883991) | (0.61775, | 5.00967)* |
| | 4 Hours Post-Dose | 3 | 3.38023 | (1.494311) | (−0.33185, | 7.09230) |
| | 5 Hours Post-Dose | 3 | 4.07789 | (1.938972) | (−0.73879, | 8.89456) |
| | 6 Hours Post-Dose | 3 | 4.68637 | (2.236808) | (−0.87017, | 10.24291) |
| | 8 Hours Post-Dose | 3 | 5.30534 | (2.311221) | (−0.43605, | 11.04673) |
| | 12 Hours Post-Dose | 3 | 4.74932 | (2.204810) | (−0.72774, | 10.22637) |
| | 24 Hours Post-Dose | 3 | 3.54284 | (1.396901) | (0.07275, | 7.01293)* |
| Day 31 | Day 31, Pre-Dose | 2 | 4.12064 | (2.778532) | NA | |
| Day 32 | Day 32, Pre-Dose | 1 | 8.13112 | (NA) | NA | |
| Day 33 | Day 33, Pre-Dose | 2 | 4.23079 | (4.113829) | NA | |
| Day 34 | Day 34, Pre-Dose | 2 | 4.10254 | (3.872363) | NA | |
| Day 35 | Day 35, Pre-Dose | 2 | 4.49045 | (4.525546) | NA | |
| Day 36 | Day 36, Pre-Dose | 2 | 3.46521 | (3.015547) | NA | |
| Day 37 | Day 37, Pre-Dose | 2 | 4.16785 | (4.069319) | NA | |
| Day 38 | Day 38, Pre-Dose | 1 | 7.64328 | (NA) | NA | |
| Day 39 | −0.5 Hours | 3 | 3.41170 | (3.688041) | (−5.74990, | 12.57330) |
| | 2 Hours Post-Dose | 3 | 3.81561 | (3.942598) | (−5.97835, | 13.60957) |
| | 4 Hours Post-Dose | 3 | 5.57811 | (5.922419) | (−9.13399, | 20.29022) |
| | 5 Hours Post-Dose | 3 | 6.26003 | (6.244712) | (−9.25269, | 21.77276) |
| | 6 Hours Post-Dose | 3 | 6.65869 | (5.703557) | (−7.50973, | 20.82711) |
| | 8 Hours Post-Dose | 3 | 7.31963 | (6.096329) | (−7.82449, | 22.46375) |
| | 12 Hours Post-Dose | 3 | 6.55378 | (4.695286) | (−5.10996, | 18.21752) |
| | 24 Hours Post-Dose | 3 | 4.54474 | (3.676985) | (−4.58940, | 13.67888) |

TABLE 12
TTM Elimination of Copper via Feces: Increase from Baseline in
Average Daily Fecal Copper Excretion (mg) (Full Analysis Set)

| | Change from Baseline | | | |
|---|---|---|---|---|
| Study Treatment Days (PK Dosing Epoch) | Mean (SD) | | (95% CI) | |
| Days 1-8 (Accumulation 15 mg/day) | 0.3278 | (0.31900) | (−0.4646, | 1.1202) |
| Days 25-28 (Steady State 15 mg/day) | 0.4931 | (0.49554) | (−0.7379, | 1.7241) |
| Days 31-35 (Accumulation 30 mg/day) | 0.3879 | (0.41929) | (−0.6537, | 1.4295) |
| Days 36-39 (Steady State 30 mg/day) | 0.0899 | (0.20206) | (−0.4120, | 0.5918) |

TABLE 12-1
TTM Elimination of Copper via Urine: Increase from Baseline in
Average Daily Urine Copper Excretion (mg) (Full Analysis Set)

| | Change from Baseline | | | |
|---|---|---|---|---|
| Study Treatment Days (PK Dosing Epoch) | Mean (SD) | | (95% CI) | |
| Days 1-8 (Accumulation 15 mg/day) | −0.0108 | (0.01119) | (−0.0386, | 0.0170) |
| Days 25-28 (Steady State 15 mg/day) | 0.1042 | (0.08556) | (−0.1084, | 0.3167) |
| Days 31-35 (Accumulation 30 mg/day) | 0.0744 | (0.05427) | (−0.0604, | 0.2092) |
| Days 36-39 (Steady State 30 mg/day) | 0.1042 | (0.04328) | (−0.0033, | 0.2118) |

Per protocol, the dose of BC-TTM was to be increased from 15 mg daily (Days 1-28) to 30 mg daily from Days 29-39. In 2 participants, this dose escalation was not carried out due to elevation of liver enzymes (maximum Grade 2 on Common Terminology Criteria for Adverse Events [CTCAE] toxicity scale). Participant 0344-1001 received 15 mg daily from Days 1-31, followed by 15 QOD from Days 32-39. Participant 0344-1003 received 15 mg daily from Days 1-24, followed by 15 mg QOD from Days 25-39. The lower mean daily dose of TTM in Period 2 may explain the smaller change in dNCC for Days 31-35 and 36-39.

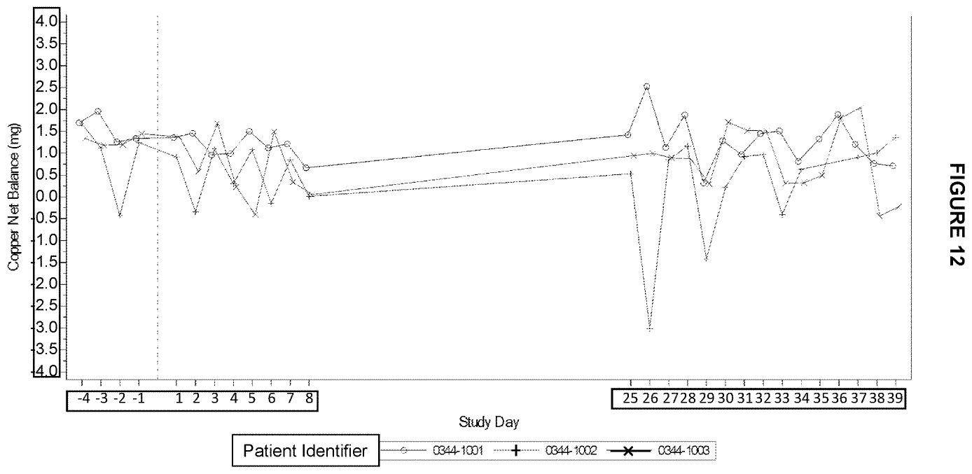

The increased mobilization of copper by TTM is evidenced by the decrease from baseline in net copper balance displayed in Table 13 and FIG. 12 (where net balance=copper input−copper output).

TABLE 13
TTM Decreased Accumulation of Copper: Decrease from Baseline
in Average Daily Net Copper Balance (mg) (Full Analysis Set)

| | Change from Baseline | |
|---|---|---|
| Study Treatment Period Days (Dosing) | Mean (SD) | (95% CI) |
| Days 1-8 (Accumulation 15 mg/day) | −0.4848(0.11902) | (−0.7805, −0.1892)* |
| Days 25-28 (Steady State 15 mg/day) | −0.6381 (0.76730) | (−2.5442, 1.2680) |
| Days 31-35 (Accumulation 30 mg/day) | −0.3925 (0.05781) | (−0.5362, −0.2489)* |
| Days 36-39 (Steady State 30 mg/day) | −0.2765 (0.31378) | (−1.0560, 0.5030) |
| Days 1-39 | −0.44799 (0.146889) | (−0.81288, −0.08310)* |

### Key Secondary Endpoints of Study 301

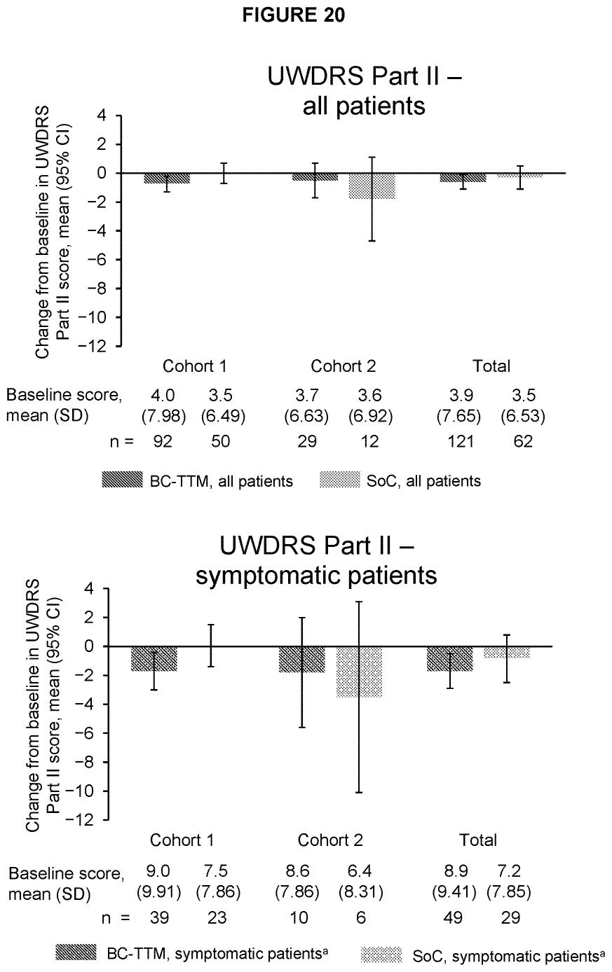

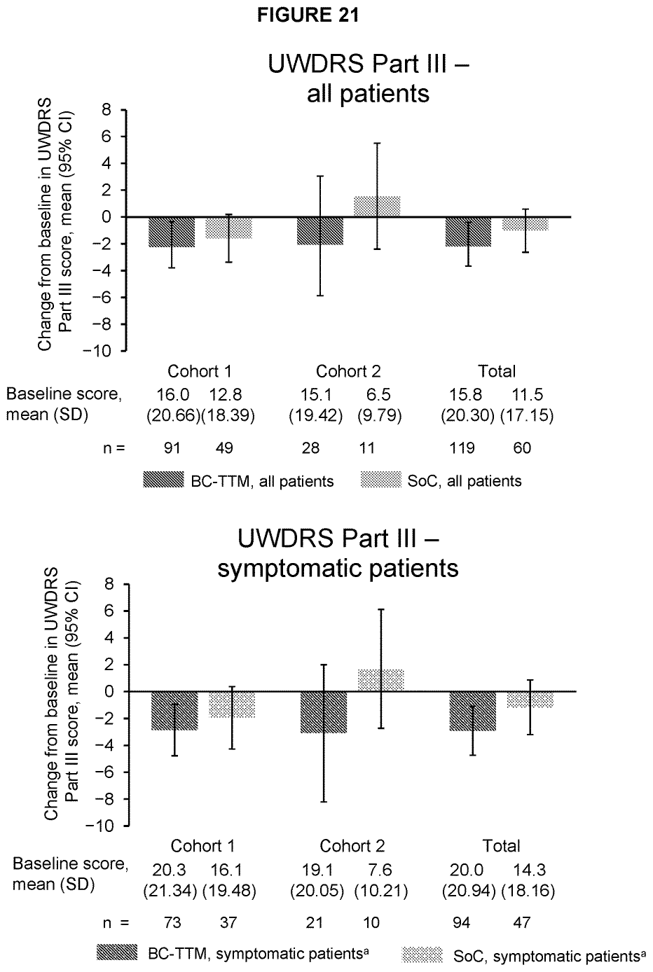

The key secondary endpoints of Study 301, meant to provide evidence of direct clinical benefit, were:
Change from baseline in the UWDRS Part II total score (activities of daily living)
Change from baseline in the UWDRS Part III Functional Subscale
Change from baseline in the UWDRS Part III individual functional items: arising from a chair, gait, speech, and handwriting
   After adjusting for multiplicity using hierarchical testing, none of the key secondary efficacy endpoints were statistically significantly different between TTM and SoC, as demonstrated in Table 14. There were trends toward stability/improvement for both treatment groups. A possible explanation for the relative lack of improvement in neurology scores is the good baseline status of the majority of patients, as demonstrated by data in Table 15. As approximately 50% of patients had baseline UWRS scores of 0, and those patients that did have scores greater than zero were relatively low in the respective UWDRS Part II (score range: 0-40) and UWRS Part III (score range: 0-175) scale range, there was little room for improvement. Additional UWDRS scores, by cohort, were presented in FIG. 20 and FIG. 21.

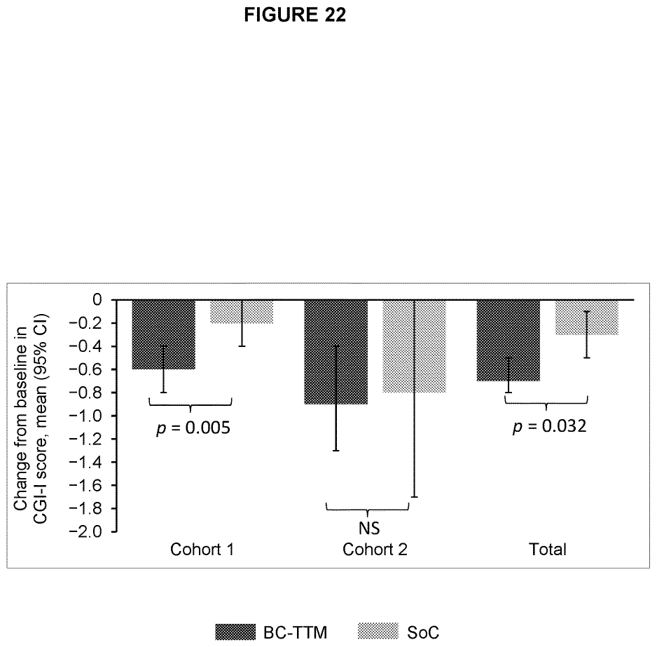

While transformed CGI-I (TCGI-I) assesses how the WD patients' condition changed compared to baseline, CGI-I may indicate the overall clinical improvement of the patients in the study compared to baseline for each treatment arm. Analysis outside of the multiplicity testing sequence showed significant improvements in transformed CGI-I scores at week 48 in cohort 1 and overall, as illustrated in FIG. 22.

TABLE 14
UWDRS Scores on TTM and SoC; Full Analysis Set

| | TTM Treatment, N = 137 | | | | SoC Treatment, N = 70 | | | | |
|---|---|---|---|---|---|---|---|---|---|
| | | | Change from | | | | Change from | | |
| UWDRS Measure | | | Baseline at | | | | Baseline at | | Difference |
| (scoring range) | Baseline | | 48 weeks | | Baseline | | 48 weeks | | at 48 weeks |
| Part II (0-40) | | | | | | | | | |
| Mean (SD) | 3.9 | (7.65) | −0.6 | (2.86) | 3.5 | (6.53) | −0.3 | (2.94) | |

| | | | | | | |
|---|---|---|---|---|---|---|
| LSM (SE) | −0.1 | (0.30) | −0.5 | (0.42) | 0.4 | (0.51) |
| 95% CI | (−0.7, | 0.5) | (−1.3, | 0.3) | (−0.6, | 1.4) |

| P-value | | | | | | | | | 0.4430 |
|---|---|---|---|---|---|---|---|---|---|
| Part III (0-175) | | | | | | | | | |
| Mean (SD) | 15.75 | (20.300) | −2.20 | (8.038) | 11.53 | (17.150) | −1.02 | (6.207) | |

| | | | | | | |
|---|---|---|---|---|---|---|
| LSM (SE) | −0.60 | (0.808) | −0.50 | (1.111) | −0.09 | (1.322) |
| 95% CI | (−2.19, | 1.00) | (−2.70, | 1.69) | (−2.70, | 2.52) |

| P-value | | | | | | | | | 0.9441 |
|---|---|---|---|---|---|---|---|---|---|
| Part III functional | | | | | | | | | |
| subscale (0-10) | | | | | | | | | |
| Mean (SD) | 1.523 | (2.1772) | −0.147 | (0.7796) | 1.269 | (2.1127) | −0.042 | (0.5383) | |

| | | | | | | |
|---|---|---|---|---|---|---|
| LSM (SE) | 0.001 | (0.0814) | 0.068 | (0.1126) | −0.067 | (0.1344) |
| 95% C | (−0.160, | 0.163) | (−0.154, | 0.291) | (−0.333, | 0.199) |

| P-value | | | | | | | | | 0.6191 |
|---|---|---|---|---|---|---|---|---|---|
| Part III-arising from | | | | | | | | | |
| chair (0-4) | | | | | | | | | |
| Mean (SD) | 0.3 | (0.90) | 0.0 | (0.41) | 0.3 | (0.91) | −0.1 | (0.50) | |

| | | | | | | |
|---|---|---|---|---|---|---|
| LSM (SE) | 0.0 | (0.04) | 0.0 | (0.05) | 0.0 | (0.06) |
| 95% CI | (−0.1, | 0.1) | (−0.1, | 0.1) | (−0.1, | 0.1) |

| P-value | | | | | | | | | 0.6869 |
|---|---|---|---|---|---|---|---|---|---|
| Part III-gait (0-10) | | | | | | | | | |
| Mean (SD) | 1.51 | (2.477) | −0.06 | (1.238) | 1.32 | (2.397) | 0.04 | (1.079) | |

| | | | | | | |
|---|---|---|---|---|---|---|
| LSM (SE) | 0.06 | (0.119) | 0.21 | (0.164) | −0.15 | (0.192) |
| 95% CI | (−0.18, | 0.29) | (−0.12, | 0.53) | (−0.53, | 0.23) |

| P-value | | | | | | | | | 0.4325 |
|---|---|---|---|---|---|---|---|---|---|
| Part III-speech (0-4) | | | | | | | | | |
| Mean (SD) | 0.9 | (1.16) | −0.1 | (0.51) | 0.7 | (1.05) | 0.0 | (0.39) | |

| | | | | | | |
|---|---|---|---|---|---|---|
| LSM (SE) | 0.0 | (0.04) | 0.0 | (0.06) | 0.0 | (0.07) |
| 95% CI | (−0.1, | 0.0) | (−0.1, | 0.1) | (−0.2, | 0.1) |

| P-value | | | | | | | | | 0.7238 |
|---|---|---|---|---|---|---|---|---|---|
| Part III-handwriting (0-4) | | | | | | | | | |
| Mean (SD) | 0.7 | (1.00) | −0.1 | (0.63) | 0.5 | (0.94) | 0.0 | (0.47) | |

| LSM (SE) | | −0.1 | (0.05) | | 0.0 | (0.07) | −0.1 | (0.09) |
|---|---|---|---|---|---|---|---|---|
| 95% CI | | (−0.2, | 0.0) | | (−0.2, | 0.1) | (−0.2, | 0.1) |
| P-value | | | | | | | 0.5077 | |
| T-CGI-I (−3 to 3, −3 is | N/A | | | N/A | | | | |
| very much improved) | | | | | | | | |
| Mean (SD) | | −0.7 | (0.93) | | −0.3 | (0.95) | | |
| LSM (SE) | | | | | | | −0.3 | (0.15) |
| 95% CI | | −0.7 | (0.09) | | −0.3 | (0.12) | (−0.6, | 0.0) |

| P-value | | | (−0.8, | −0.5) | | | (−0.6, | −0.1) | 0.0316 |
|---|---|---|---|---|---|---|---|---|---|
| CGI-S (1-7, 1 is normal) | | | | | | | | | |
| Mean (SD) | 2.9 | (1.60) | −0.5 | (0.88) | 2.5 | (1.41) | −0.1 | (0.85) | |

| LSM (SE) | | | −0.1 | (0.11) | −0.2 | (0.13) |
|---|---|---|---|---|---|---|
| 95% CI | −0.3 | (0.08) | (−0.4, | 0.1) | (−0.4, | 0.1) |

| | | | |
|---|---|---|---|
| P-value | (−0.5, | −0.2) | 0.2290 |

Abbreviation:
SoC = standard of care;
SD = standard deviation;
LSM = least squared mean;
SE = standard error;
CI = confidence interval;
UWDRS = Unified Wilson's Disease Rating Scale;
T-CGI-I = Transformed Clinical Global Impression of Improvement,
CGI-S = Clinical Global Impression of Severity, negative change means improvement

Another possible explanation for the relative lack of significant change in neurology scores is the low total baseline UWDRS scores in the overall population (data in Table 15). Overall, 55.9% and 51.4% of TTM and SoC participants, respectively, had a baseline UWDRS Part 11 total score of 0, and 21.3% and 18.6% of TTM and SoC participants, respectively, had a baseline UWDRS Part III total score of 0. Combined, 19.4% (40/206) of participants had a baseline total UWDRS score of 0 on both Part 11 and Part III. The impact of this is low overall population means for Part 11 (3.5 to 4.0 on a 40-point scale) and Part III (6.50 to 15.97 on a 175-point scale). At the population comparison level, there was little room for overall improvement; however, at the individual participant level changes in scores on individual items could potentially capture meaningful clinical changes for the individual participant over time.

TABLE 15
UWDRS Part II Baseline Scores on TTM and SoC (Range 0-40); Full Analysis Set

| UWDRS Measure | Visit | Cohort 1 | | | | Cohort 2 | | | | Overall | | | |
|---|---|---|---|---|---|---|---|---|---|---|---|---|---|
| (Scoring Range) | Statistic | TTM | | SoC | | TTM | | SoC | | TTM | | SoC | |
| Part II | No. patients | 103 | | 56 | | 33 | | 14 | | 136 | | 70 | |
| (0-40) | Mean (SD) | 4.0 | (7.98) | 3.5 | (6.49) | 3.7 | (6.63) | 3.6 | (6.92) | 3.9 | (7.65) | 3.5 | (6.53) |
| | Median (IQR) | 0.0 | (3.0) | 0.0 | (3.0) | 0.0 | (3.0) | 1.0 | (4.0) | 0.0 | (3.0) | 0.0 | (3.0) |
| | Q1, Q3 | 0.0, | 3.0 | 0.0, | 3.0 | 0.0, | 3.0 | 0.0, | 4.0 | 0.0, | 3.0 | 0.0, | 3.0 |
| | No. (%) with | 57 | (55.3%) | 30 | (53.6%) | 19 | (57.6%) | 6 | (42.9%) | 76 | (55.9%) | 36 | (51.4%) |
| | score of 0 | | | | | | | | | | | | |
| | No. (%) with | 46 | (44.7%) | 26 | (46.4%) | 14 | (42.4%) | 8 | (57.1%) | 60 | (44.1%) | 34 | (48.6%) |
| | score of >0 | | | | | | | | | | | | |
| Part III | No. patients | 103 | | 56 | | 33 | | 14 | | 136 | | 70 | |
| (0-175) | Mean (SD) | 15.97 | (20.664) | 12.79 | (18.392) | 15.06 | (19.415) | 6.50 | (9.788) | 15.75 | (20.300) | 11.53 | (17.150) |
| | Median (IQR) | 7.00 | (23.00) | 5.00 | (11.00) | 7.00 | (17.00) | 2.00 | (3.00) | 7.00 | (21.00) | 4.50 | (11.00) |
| | Q1, Q3 | 1.00, | 24.00 | 2.00, | 13.00 | 2.00, | 19.00 | 2.00, | 5.00 | 1.00, | 22.00 | 2.00, | 13.00 |
| | No. (%) with | 22 | (21.4%) | 11 | (19.6%) | 7 | (21.2%) | 2 | (14.3%) | 29 | (21.3%) | 13 | (18.6%) |
| | score of 0 | | | | | | | | | | | | |
| | No. (%) with | 81 | (78.6%) | 45 | (80.4%) | 26 | (78.8) | 12 | (85.7) | 107 | (78.7%) | 57 | (81.4%) |
| | score of >0 | | | | | | | | | | | | |

Abbreviations: SoC = standard of care; No. = number of participants; SD = standard deviation; IQR = inter-quartile range; Q1, Q3 = quartile 1, quartile 3

A modest reduction from baseline was seen in UWDRS scores 0-48W for symptomatic patients: Part II score change (mean) was −1.7 TTM and −0.8 SoC; Part III score change (mean [95% Cl]) was −2.91 [−4.74, −1.09] TTM and −1.17 [−3.20, 0.86] SoC. Table 15-1 summarizes the UWDRS Part III score change at 48W for the patients who were symptomatic at baseline.

TABLE 15-1
UWDRS Part III Baseline Scores on TTM and SoC; Full Analysis Set

| Visit | Cohort 1 | | Cohort 2 | | Overall | |
|---|---|---|---|---|---|---|
| Statistic | TTM | SoC | TTM | SoC | TTM | SoC |
| No. patients | 81 | 44 | 26 | 12 | 107 | 56 |

Week 48, Change from Baseline

| N | 73 | | 37 | | 21 | | 10 | | 94 | | 47 | |
|---|---|---|---|---|---|---|---|---|---|---|---|---|
| Mean | −2.86 | (8.206) | −1.95 | (6.984) | −3.10 | (11.207) | 1.70 | (6.183) | 2.91 | (8.897) | −1.17 | (6.923) |
| (SD) | | | | | | | | | | | | |
| Median | −2.00 | (7.00) | −2.00 | (4.00) | −3.00 | (9.00) | −1.00 | (3.02) | −2.00 | (7.00) | −1.00 | (4.00) |
| (IQR) | | | | | | | | | | | | |
| Q1, Q3 | −6.00, | 1.00 | −4.00, | 0.00 | −8.00, | 1.00 | −1.00, | 2.02 | −6.00, | 1.00 | −3.00, | 1.00 |
| Min, Max | −28.0, | 31.0 | −22.0, | 11.0 | −34.0, | 20.0 | −5.0, | 17.0 | −34.0, | 31.0 | −22.0, | 17.0 |
| 95% CI | (−4.78, | −0.95) | (−4.27, | 0.38) | (−8.20, | 2.01) | (−2.72, | 6.13) | (−4.74, | −1.09) | (−3.20, | 0.86) |
| LSM (SE) | −2.68 | (0.885) | −1.89 | (1.207) | 1.43 | (2.748) | 1.37 | (4.119) | −1.54 | (0.954) | −1.21 | (1.325) |
| 95% CI | (−4.43, | −0.93) | (−4.28, | 0.50) | (−4.25, | 7.10) | (−7.13, | 9.88) | (−3.43, | 0.35) | (−3.83, | 1.41) |
| LSM | −0.79 | (1.498) | | | 0.05 | (5.041) | | | −0.33 | (1.637) | | |
| Diff (SE) | | | | | | | | | | | | |
| 95% CI | (−3.76, | 2.18) | | | (−10.36, | 10.46) | | | (−3.57, | 2.91) | | |

| | | | |
|---|---|---|---|
| P-value | 0.5997 | 0.9921 | 0.8420 |

Abbreviations: SoC = standard of care; No. = number of participants; SD = standard deviation; IQR = inter-quartile range; Q1, Q3 = quartile 1, quartile 3
LSM and its 95% CI and 2-sided p-value are calculated using an REML based MMRM with fixed effects for baseline, cohort, visit, and cohort-by-visit interaction. For cohort analysis, analysis was performed on each cohort removing cohort and cohort-by-visit interaction. The Kenward-Rodger approximation is used to estimate denominator degrees of freedom. CI's and MMRM estimates are only displayed when n ≥ 3.

The symptoms and the associated impacts of WD generally fall into neurologic, psychiatric, and hepatic categories. Previous work, including concept elicitation interviews with WD patients, has provided insight into the complexity and heterogeneity among patients. Consistent with this, participants enrolled in Study 301 presented with a wide array of symptoms and these have different types of impacts on their lives.

Exit interviews were conducted with a sample of participants (n=10) to understand their experiences with TTM treatment. These exit interviews reflect the diversity in neurologic dysfunction and the need to better understand treatment experience with a review of the participant level data. Overall, the qualitative information suggests that participants who were symptomatic at the start of treatment with TTM experienced improvements or maintenance across several neurologic symptoms (including walking, balance, speech and tremor) that were considered meaningful. Conversely, participants who did not report experiencing any symptoms at the start of the study did not develop any symptoms while on treatment and considered any worsening (experiencing symptoms or deterioration) as meaningful. The exit interview insights provide deeper insight into the experience of the participants interviewed and add to the observation that at the population comparison level there is little room for overall improvement.

Data from participants who have taken TTM for longer than 48 weeks are being analyzed to assess the longer-term safety and efficacy (continued control of copper and clinical benefit) of this therapy. This includes 19 participants from Phase 2 Study 201 who transferred into the Study 301 Extension and have received treatment with TTM for more than 5 years, as well as participants who enrolled into Study 301 and were randomized and treated with TTM either in the Primary Evaluation Period or the open-label extension. Pooled results from all such participants will be integrated together into one timeline to allow analyses of changes in UWDRS, long-term copper control, and long-term safety laboratory data.

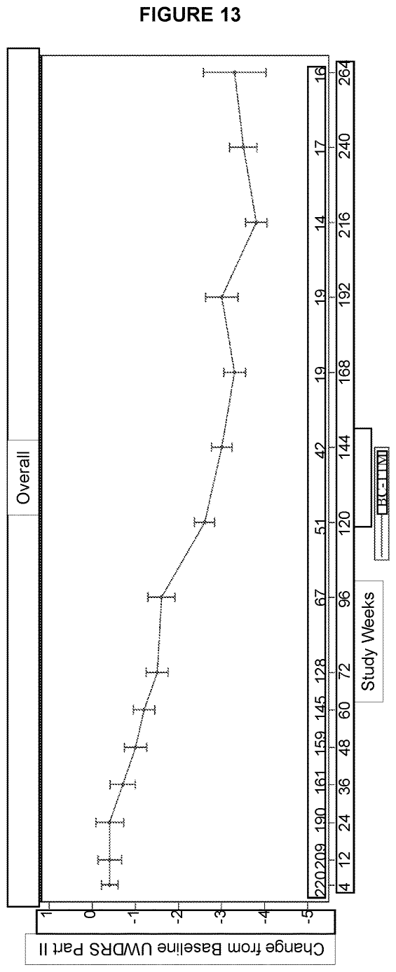

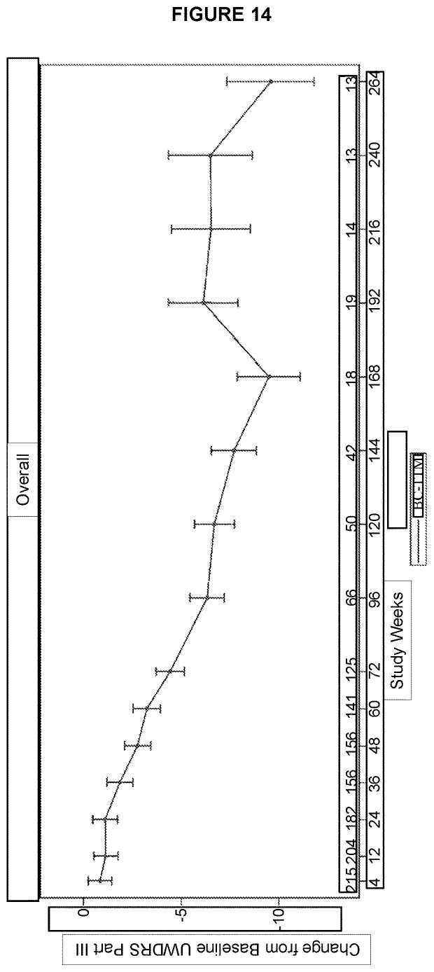

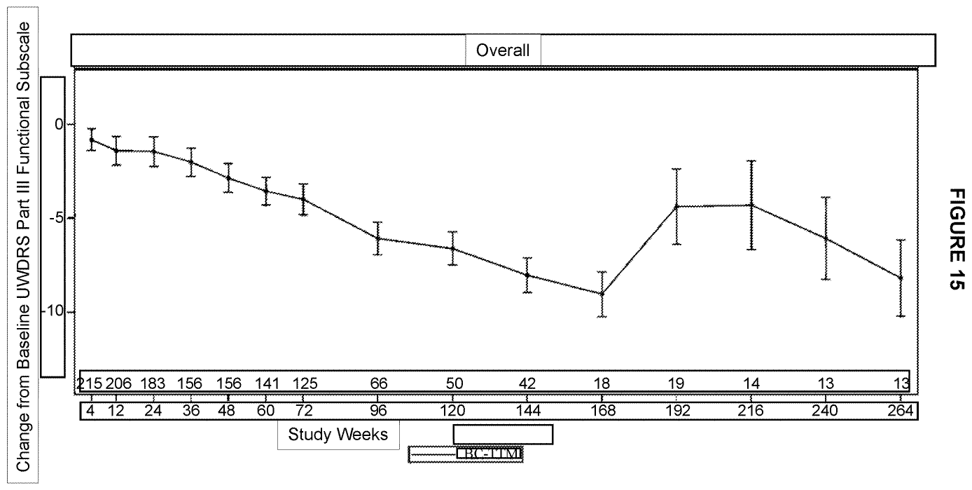

The UWDRS Part II total score results for this pooled population are presented in Table 16 and FIG. 13 and the UWDRS Part III total score results are in Table 17 and FIG. 14. The UWDRS Part III Functional Subscale (comprised of the items arising from a chair, gait, handwriting and speech) results for this pooled population are presented in Table 18 and FIG. 15. All tables document decreases in the mean and least squares mean (LSM) values over time, with generally greater decreases at the later timepoints. This indicates an improvement in the overall population, as lower scores denote improvement in signs and symptoms. After 36 weeks of exposure to TTM, significance in mean and LSM is reached (0 is excluded from the mean and LSM 95% Cis) and maintained in virtually every instance. The graphical representations show lines sloping downward over time, indicating a slow but steady improvement during treatment with TTM. These results indicate that participant reported UWDRS Part II scores and clinical/neurologist reported UWDRS Part III and Part III Functional Subscale scores all demonstrate continuing improvement over up to 5 years of TTM treatment, evidenced by statistically significant reductions from baseline.

TABLE 16
Improvement in UWDRS Part II Scores (Range 0-40) over 5 Years of TTM Treatment
from Baseline (Pooling Full Analysis Set, Studies 301 and 201)

| TTM Exposure | | Mean | Mean | LSM | LSM |
|---|---|---|---|---|---|
| (Weeks) | N | (SD) | 95% CI | (SE) | 95% CI |
| Baseline | 222 | 4.0 (7.73) | (3.0, 5.0) | NA | NA |
| 4 | 220 | −0.3 (2.28) | (−0.6, 0.0) | −0.4 (0.19) | (−0.8, 0.0) |
| 12 | 209 | −0.4 (2.76) | (−0.8, 0.0) | −0.4 (0.27) | (−0.9, 0.1) |
| 24 | 190 | −0.4 (3.42) | (−0.9, 0.1) | −0.4 (0.32) | (−1.1, 0.2) |
| 36 | 161 | −0.9 (4.11) | (−1.6, −0.3)* | −0.7 (0.29) | (−1.3, −0.2)* |
| 48 | 159 | −1.2 (4.24) | (−1.9, −0.6)* | −1.0 (0.26) | (−1.5, −0.5)* |
| 60 | 145 | −1.4 (4.35) | (−2.1, −0.7)* | −1.2 (0.25) | (−1.7, −0.7)* |
| 72 | 128 | −1.9 (4.74) | (−2.7, −1.0)* | −1.5 (0.25) | (−2.0, −1.0)* |
| 96 | 67 | −2.5 (6.11) | (−4.0, −1.0)* | −1.6 (0.31) | (−2.2, −1.0)* |
| 120 | 51 | −3.1 (6.82) | (−5.0, −1.2)* | −2.6 (0.23) | (−3.0, −2.1)* |
| 144 | 42 | −3.3 (6.61) | (−5.3, −1.2)* | −3.0 (0.24) | (−3.5, −2.6)* |
| 168 | 19 | −6.2 (10.17) | (−11.1, −1.3)* | −3.3 (0.25) | (−3.8, −2.7)* |
| 192 | 19 | −5.9 (9.88) | (−10.7, −1.1)* | −3.0 (0.38) | (−3.8, −2.2)* |
| 216 | 14 | −7.1 (10.90) | (−13.4, −0.8)* | −3.8 (0.25) | (−4.3, −3.3)* |
| 240 | 17 | −6.6 (10.09) | (−11.8, −1.4)* | −3.5 (0.32) | (−4.2, −2.8)* |
| 264 | 16 | −6.2 (9.02) | (−11.0, −1.4)* | −3.3 (0.73) | (−4.8, −1.8)* |

Note:
Baseline contains observed values and other timepoints are change from baseline.
Note:
LSM and its 95% CI are calculated using an REML based MMRM with fixed effects for visit, cohort, baseline-by-visit interaction, and baseline value as a covariate. The Kenward-Roger approximation is used to estimate degrees of freedom. MMRM was performed for each cohort subset using the same fixed effect terms in the model except for removing cohort term.
Note:
*= statistically significant with two-sided alpha = 0.05 since 95% CI does not contain 0.
Note:
For UWDRS, negative change means improvement.

TABLE 17
Improvement in UWDRS Part III (Range 0-175) over 5 Years of
TTM from Baseline (Pooling Full Analysis Set, 301 and 201)

| TTM Exposure | | Mean | Mean | LSM | LSM |
|---|---|---|---|---|---|
| (Wks) | N | (SD) | 95% CI | (SE) | 95% CI |
| Baseline | 220 | 15.43 (19.833) | (12.79, 18.06) | NA | NA |
| 4 | 215 | −0.72 (6.701) | (−1.62, 0.18) | −0.83 (0.607) | (−2.03, 0.36) |
| 12 | 204 | −1.11 (7.065) | (−2.08, −0.13)* | −1.13 (0.612) | (−2.34, 0.07) |
| 24 | 182 | −0.97 (8.447) | (−2.21, 0.26) | −1.10 (0.629) | (−2.34, 0.13) |
| 36 | 156 | −2.38 (8.581) | (−3.73, −1.02)* | −1.85 (0.657) | (−3.14, −0.56)* |
| 48 | 156 | −3.37 (9.778) | (−4.91, −1.82)* | −2.76 (0.675) | (−4.09, −1.43)* |
| 60 | 141 | −2.00 (6.40) | (−5.58, −2.18)* | −3.24 (0.700) | (−4.62, −1.87)* |
| 72 | 125 | −4.93 (11.682) | (−7.00, −2.86)* | −4.43 (0.734) | (−5.87, −2.99)* |
| 96 | 66 | −7.95 (14.078) | (−11.41, −4.49)* | −6.32 (0.881) | (−8.05, −4.59)* |
| 120 | 50 | −8.92 (15.045) | (−13.19, −4.64)* | −6.69 (1.027) | (−8.71, −4.68)* |
| 144 | 42 | −9.78 (15.423) | (−14.58, −4.97)* | −7.70 (1.153) | (−9.97, −5.44)* |
| 168 | 18 | −18.08 (19.127) | (−27.59, −8.57)* | −9.48 (1.616) | (−12.66, −6.31)* |
| 192 | 19 | −12.65 (19.476) | (−22.04, −3.27)* | −6.13 (1.783) | (−9.63, −2.63)* |
| 216 | 14 | −16.17 (19.323) | (−27.33, −5.01)* | −6.52 (2.013) | (−10.47, −2.56)* |
| 240 | 13 | −11.48 (16.473) | (−21.43, −1.52)* | −6.49 (2.137) | (−10.68, −2.29)* |
| 264 | 13 | −14.00 (16.513) | (−23.98, −4.02)* | −9.57 (2.222) | (−13.93, −5.21)* |

Note:
Baseline contains observed values and other timepoints are change from baseline.
Note:
LSM and its 95% CI are calculated using an REML based MMRM with fixed effects for visit, cohort, baseline-by-visit interaction, and baseline value as a covariate. The Kenward-Roger approximation is used to estimate degrees of freedom. MMRM was performed for each cohort subset using the same fixed effect terms in the model except for removing cohort term.
Note:
*= statistically significant with two-sided alpha = 0.05 since 95% CI does not contain 0.
Note:
For UWDRS, negative change means improvement.

TABLE 18
Improvement in UWDRS Part III Functional Subscale Scores (Range 0-10) over 5
Years of TTM from Baseline (Pooling Full Analysis Set, Studies 301 and 201)

| TTM Exposure | | Mean | Mean | LSM | LSM |
|---|---|---|---|---|---|
| (Wks) | N | (SD) | 95% CI | (SE) | 95% CI |
| Baseline | 220 | 1.590 (2.2640) | (1.289, 1.890) | NA | NA |
| 4 | 215 | −0.072 (0.6009) | (−0.153, 0.009) | −0.083 (0.0575) | (−0.197, 0.030) |
| 12 | 206 | −0.137 (0.7864) | (−0.245, −0.029)* | −0.141 (0.0753) | (−0.289, 0.008) |
| 24 | 183 | −0.157 (0.9091) | (−0.290, −0.025)* | −0.146 (0.0789) | (−0.301, 0.010) |
| 36 | 156 | −0.286 (0.9929) | (−0.443, −0.129)* | −0.202 (0.0762) | (−0.352, −0.051)* |
| 48 | 156 | −0.345 (1.0940) | (−0.518, −0.172)* | −0.285 (0.0759) | (−0.434, −0.135)* |
| 60 | 141 | −0.422 (1.0814) | (−0.602, −0.242)* | −0.355 (0.0737) | (−0.500, −0.209)* |
| 72 | 125 | −0.494 (1.2826) | (−0.721, −0.267)* | −0.399 (0.0814) | (−0.559, −0.239)* |
| 96 | 66 | −0.900 (1.5193) | (−1.273, −0.527)* | −0.607 (0.0882) | (−0.781, −0.433)* |
| 120 | 50 | −0.946 (1.7746) | (−1.450, −0.442)* | −0.660 (0.0890) | (−0.836, −0.483)* |
| 144 | 42 | −1.042 (1.7227) | (−1.579, −0.505)* | −0.803 (0.0922) | (−0.986, −0.620)* |
| 168 | 18 | −1.841 (2.1818) | (−2.926, −0.756)* | −0.903 (0.1189) | (−1.142, −0.664)* |
| 192 | 19 | −1.250 (2.1459) | (−2.284, −0.216)* | −0.437 (0.2007) | (−0.849, −0.032)* |
| 216 | 14 | −1.519 (2.3987) | (−2.904, −0.134)* | −0.430 (0.2362) | (−0.913, 0.053) |
| 240 | 13 | −0.898 (1.3384) | (−1.707, −0.090)* | −0.607 (0.2176) | (−1.054, −0.160)* |
| 264 | 13 | −0.995 (1.2369) | (−1.742, −0.247)* | −0.818 (0.2025) | (−1.242, −0.394)* |

Note:
Subscale recommended by FDA outcomes group, includes speech, handwriting, arising from chair, and gait.
Note:
Baseline contains observed values and other timepoints are change from baseline.
Note:
LSM and its 95% CI are calculated using an REML based MMRM with fixed effects for visit, cohort, baseline-by-visit interaction, and baseline value as a covariate. The Kenward-Roger approximation is used to estimate degrees of freedom. MMRM was performed for each cohort subset using the same fixed effect terms in the model except for removing cohort term.
Note:
*= statistically significant with two-sided alpha = 0.05 since 95% CI does not contain 0
Note:
For UWDRS, negative change means improvement.

Table 18-1 provides some interim long term safety and efficacy results through the extension period of Study 301. These results represent an integrated data set of Study 301 participants who either started on TTM or transitioned to TTM after the 48-week Primary Period. They demonstrate statistically significant improvements from baseline in key secondary endpoints, such as UWDRS and CGI, in patients treated with TTM. In addition, these results demonstrate that the level of improvement increases with continuing TTM treatment overtime.

TABLE 18-1
TTM Treatment Long Term Safety and Efficacy Set (Study 301)

| | TTM Treatment Long Term Safety and Efficacy Set N = 178 | | | | | | |
|---|---|---|---|---|---|---|---|
| Measure | | Change from Baseline at Week | | | | | |
| (scoring range) | Baseline | 24 | | 48 | | 72 | |
| UWDRS Part II (0-40) | | | | | | | |
| N | 174 | 155 | | 131 | | 108 | |
| Mean (SD) | 3.4 | −0.3 | (1.99) | −0.7 | (2.70) | −1.2 | (2.92) |
| 95% CI | (7.04) | (−0.6, | 0.0) | (−1.2, | −0.3)* | (−1.7, | −0.6)* |
| LSM (SE) | | −0.4 | (0.20) | −0.7 | (0.21) | −1.0 | (0.22) |
| 95% CI | | (−0.8, | 0.0) | (−1.1, | −0.3)* | (−1.4, | −0.6)* |
| UWDRS Part III (0-175) | | | | | | | |
| N | 172 | 148 | | 128 | | 105 | |
| Mean (SD) | 13.99 | −0.74 | (5.937) | −2.32 | (7.735) | −3.27 | (9.840) |
| 95% CI | (19.113) | (−1.70, | 0.22) | (−3.67, | −0.96)* | (−5.18, | −1.37)* |
| LSM (SE) | | −1.32 | (0.619) | −2.47 | (0.638) | −3.47 | (0.672) |
| 95% CI | | (−2.53, | −0.10)* | (−3.73, | −1.22)* | (−4.79, | −2.15)* |
| UWDRS Part III functional | | | | | | | |
| subscale (0-10) | | | | | | | |
| N | 172 | 149 | | 128 | | 105 | |
| Mean (SD) | 1.390 | −0.139 | (0.4540) | −0.235 | (0.6976) | −0.290 | (0.8908) |
| 95% CI | (2.1420) | (−0.212, | −0.065)* | (−0.357, | −0.113)* | (−0.463, | −0.118)* |
| LSM (SE) | | −0.158 | (0.0547) | −0.230 | (0.0569) | −0.253 | (0.0604) |
| 95% CI | | (−0.265, | −0.050)* | (−0.341, | −0.118)* | (−0.372, | −0.135)* |
| UWDRS Part III-arising | | | | | | | |
| from chair (0-4) | | | | | | | |
| N | 172 | 149 | | 128 | | 105 | |
| Mean (SD) | 0.3 | 0.0 | (0.38) | 0.0 | (0.40) | −0.1 | (0.57) |
| 95% CI | (0.89) | (−0.1, | 0.0) | (−0.1, | 0.0) | (−0.2, | 0.0) |
| LSM (SE) | | −0.1 | (0.03) | 0.0 | (0.04) | −0.1 | (0.04) |
| 95% CI | | (−0.1, | 0.0) | (−0.1, | 0.0) | (−0.1, | 0.0) |
| UWDRS Part III- | | | | | | | |
| gait (0-4) | | | | | | | |
| N | 172 | 149 | | 128 | | 104 | |
| Mean (SD) | 1.40 | −0.17 | (1.113) | −0.18 | (1.262) | −0.12 | (1.321) |
| 95% CI | (2.406) | (−0.35, | 0.01) | (−0.40, | 0.04) | (−0.38, | 0.14) |
| LSM (SE) | | −0.21 | (0.100) | −0.26 | (0.104) | −0.15 | (0.110) |
| 95% CI | | (−0.41, | −0.02)* | (−0.47, | −0.06)* | (−0.37, | 0.07) |
| UWDRS Part III- | | | | | | | |
| speech (0-4) | | | | | | | |
| N | 172 | 149 | | 128 | | 105 | |
| Mean (SD) | 0.8 | −0.1 | (0.38) | −0.1 | (0.53) | −0.1 | (0.51) |
| 95% CI | (1.13) | (−0.1, | 0.0) | (−0.2, | 0.0) | (−0.2, | 0.0) |
| LSM (SE) | | −0.1 | (0.04) | −0.1 | (0.04) | −0.1 | (0.04) |
| 95% CI | | (−0.2, | 0.0) | (−0.2, | 0.0) | (−0.2, | −0.1)* |
| UWDRS Part III- | | | | | | | |
| handwriting (0-4) | | | | | | | |
| N | 171 | 148 | | 128 | | 105 | |
| Mean (SD) | 0.6 | −0.1 | (0.38) | −0.2 | (0.59) | −0.2 | (0.59) |
| 95% CI | (0.99) | (−0.1, | 0.0) | (−0.3, | −0.1)* | (−0.3, | −0.1)* |
| LSM (SE) | | 0.0 | (0.04) | −0.1 | (0.04) | −0.2 | (0.05) |
| 95% CI | | (−0.1, | 0.0) | (−0.2, | −0.1)* | (−0.2, | −0.1)* |
| Transformed CGI-I** | | | | | | | |
| N | n/a** | 151 | | 134 | | 106 | |
| Mean (SD) | | −0.5 | (0.82) | −0.7 | (0.91) | −0.5 | (0.86) |
| 95% CI | | (−0.6, | −0.3)* | (−0.8, | −0.5)* | (−0.6, | −0.3)* |
| LSM (SE) | | −0.6 | (0.07) | −0.8 | (0.07) | −0.6 | (0.08) |
| 95% CI | | (−0.7, | −0.4)* | (−0.9, | −0.6)* | (−0.7, | −0.4)* |
| CGI-S (1 to 7, | | | | | | | |
| 1 is normal) | | | | | | | |
| N | 172 | 147 | | 130 | | 103 | |
| Mean (SD) | 2.7 | −0.2 | (0.79) | −0.5 | (0.94) | −0.6 | (1.10) |
| 95% CI | (1.58) | (−0.4, | −0.1)* | (−0.6, | −0.3)* | (−0.8, | −0.4)* |
| LSM (SE) | | −0.3 | (0.07) | −0.4 | (0.07) | −0.5 | (0.07) |
| 95% CI | | (−0.4, | −0.2)* | (−0.6, | −0.3)* | (−0.6, | −0.3)* |

| | TTM Treatment Long Term Safety and Efficacy Set N = 178 | | | | | |
|---|---|---|---|---|---|---|
| Measure | Change from Baseline at Week | | | | | |
| (scoring range) | 96 | | 120 | | 144 | |
| UWDRS Part II (0-40) | | | | | | |
| N | 47 | | 31 | | 24 | |
| Mean (SD) | −1.1 | (3.61) | −1.4 | (3.48) | −2.0 | (4.68) |
| 95% CI | (−2.2, | −0.1)* | (−2.7, | −0.1)* | (−4.0, | 0.0) |
| LSM (SE) | −1.1 | (0.30) | −1.7 | (0.35) | −2.0 | (0.39) |
| 95% CI | (−1.7, | −0.6)* | (−2.4, | −1.0)* | (−2.7, | −1.2)* |
| UWDRS Part III (0-175) | | | | | | |
| N | 46 | | 30 | | 24 | |
| Mean (SD) | −4.09 | (10.827) | −4.08 | (11.618) | −5.99 | (12.215) |
| 95% CI | (−7.31, | −0.87)* | (−8.42, | 0.26) | (−11.14, | −0.83)* |
| LSM (SE) | −4.77 | (0.865) | −5.27 | (1.016) | −6.38 | (1.112) |
| 95% CI | (−6.47, | −3.08)* | (−7.26, | −3.27)* | (−8.56, | −4.20)* |
| UWDRS Part III functional | | | | | | |
| subscale (0-10) | | | | | | |
| N | 46 | | 30 | | 24 | |
| Mean (SD) | −0.503 | (0.9648) | −0.410 | (1.1867) | −0.573 | (1.2617) |
| 95% CI | (−0.790, | −0.217)* | (−0.853, | 0.033) | (−1.106, | −0.040)* |
| LSM (SE) | −0.386 | (0.0801) | −0.392 | (0.0952) | −0.489 | (0.1046) |
| 95% CI | (−0.543, | −0.229)* | (−0.578, | −0.205)* | (−0.694, | −0.284)* |
| UWDRS Part III-arising | | | | | | |
| from chair (0-4) | | | | | | |
| N | 46 | | 30 | | 24 | |
| Mean (SD) | −0.2 | (0.76) | −0.2 | (0.83) | −0.3 | (1.03) |
| 95% CI | (−0.4, | 0.1) | (−0.5, | 0.1) | (−0.7, | 0.2) |
| LSM (SE) | −0.1 | (0.05) | −0.1 | (0.06) | −0.2 | (0.07) |
| 95% CI | (−0.2, | 0.0) | (−0.2, | 0.0) | (−0.3, | 0.0) |
| UWDRS Part III- | | | | | | |
| gait (0-4) | | | | | | |
| N | 46 | | 29 | | 24 | |
| Mean (SD) | −0.11 | (1.390) | 0.26 | (1.683) | 0.00 | (1.950) |
| 95% CI | (−0.52, | 0.30) | (−0.38, | 0.90) | (−0.82, | 0.82) |
| LSM (SE) | −0.12 | (0.143) | 0.04 | (0.172) | −0.25 | (0.186) |
| 95% CI | (−0.40, | 0.16) | (−0.30, | 0.38) | (−0.61, | 0.12) |
| UWDRS Part III- | | | | | | |
| speech (0-4) | | | | | | |
| N | 46 | | 30 | | 24 | |
| Mean (SD) | −0.2 | (0.57) | −0.1 | (0.61) | −0.2 | (0.59) |
| 95% CI | (−0.3, | 0.0) | (−0.3, | 0.1) | (−0.5, | 0.0) |
| LSM (SE) | −0.2 | (0.05) | −0.2 | (0.06) | −0.3 | (0.07) |
| 95% CI | (−0.3, | −0.1)* | (−0.3, | 0.0) | (−0.4, | −0.1)* |
| UWDRS Part III- | | | | | | |
| handwriting (0-4) | | | | | | |
| N | 45 | | 30 | | 23 | |
| Mean (SD) | −0.4 | (0.66) | −0.5 | (0.68) | −0.5 | (0.59) |
| 95% CI | (−0.6, | −0.2)* | (−0.8, | −0.2)* | (−0.7, | −0.2)* |
| LSM (SE) | −0.3 | (0.06) | −0.3 | (0.07) | −0.3 | (0.08) |
| 95% CI | (−0.4, | −0.2)* | (−0.5, | −0.2)* | (−0.4, | −0.1)* |
| Transformed CGI-I** | | | | | | |
| N | 49 | | 33 | | 23 | |
| Mean (SD) | −0.5 | (0.98) | −0.7 | (0.92) | −0.7 | (0.76) |
| 95% CI | (−0.8, | −0.2)* | (−1.0, | −0.4)* | (−1.0, | −0.4)* |
| LSM (SE) | −0.6 | (0.10) | −0.7 | (0.12) | −0.8 | (0.14) |
| 95% CI | (−0.8, | −0.4)* | (−1.0, | −0.5)* | (−1.0, | −0.5)* |
| CGI-S (1 to 7, | | | | | | |
| 1 is normal) | | | | | | |
| N | 47 | | 33 | | 23 | |
| Mean (SD) | −0.6 | (0.97) | −0.6 | (1.06) | −0.6 | (0.78) |
| 95% CI | (−0.9, | −0.3)* | (−1.0, | −0.3)* | (−0.9, | −0.3)* |
| LSM (SE) | −0.5 | (0.09) | −0.7 | (0.11) | −0.6 | (0.12) |
| 95% CI | (−0.7, | −0.3)* | (−0.9, | −0.4)* | (−0.8, | −0.4)* |

*= statistically significant with two-sided alpha = 0.05 since 95% CI does not contain 0.
**= Clinician impression of change from baseline. Accordingly there is no baseline value. Scores transformed to range from −3 to 3 where: −3 = Very much improved, −2 = Much improved, −1 = Minimally improved, 0 = No change, 1 = Minimally worse, 2 = Much worse, 3 = Very much worse.

### Adverse Events of Special Interest (AESI):

### Neurologic Events

Acute worsening of neurologic symptoms typically within 6 months of start of therapy is a known complication associated with chelation therapy for WD. The presumed mechanism is a rapid mobilization of unbound copper resulting in higher blood NCC and triggering a cytotoxic effect in neuronal tissue with subsequent neurologic deterioration in treated patients. Neurologic AESI for Study 301 were considered as all AEs in the Medical Dictionary for Regulatory Activities (MedDRA) System Organ Class (SOC) “Nervous system disorders” or any other AE judged by the Investigator as an AESI.

Overall, neurologic AESI were reported under the SOC of “Nervous system disorders” and “Psychiatric disorders” in 47 (34.3%) and in 5 (3.6%) participants, respectively. Adverse events reported as AESI in both these SOC are described below.

### Nervous System Disorders (SOC)

The incidence of AEs reported in the SOC “Nervous system disorders” was higher in TTM treatment group compared with SoC group (34.3% vs 21.4%). The commonly reported AEs in both groups were headache (8.0% vs 8.6%) and tremor (7.3% vs 2.9%). Of the neurologic AEs, a significant difference between the treatment groups (TTM and SoC) was observed for the event of tremor. Twelve events of tremor occurred in 10 (7.3%) participants. All events were nonserious and low grade (Grade 1 and Grade 2). Of the 10 (7.3%) participants, 6 participants reported the event as a “worsening” or “increasing of tremor.” Dose was interrupted due to 3 tremor events; for 2 events the outcome was “resolved” and for 1 event was “not recovered.” For the remaining 9 events, dose was not modified and the outcome was “not recovered” for 3 events, “recovered” for 4 events and “recovering” for 2 events.

The incidence of participants with the highest severity grade AEs was similar in both treatment groups for Grade 1 (24.1% vs 21.4%) AEs, and higher in the TTM treatment group for Grade 2 AEs (13.9% vs 4.3%). No Grade 3 AEs were observed in either treatment group, and Grade 4 and Grade 5 AEs were observed in 1 participant each in the TTM treatment group. One AE (Preferred Term “neurologic deterioration”) led to treatment withdrawal.

Similar to the overall AEs, the incidence of SAEs was higher in the TTM treatment group compared with the SoC group [6 (4.4%) vs 1 (1.4%)]. Seven SAEs were reported in 6 participants. In the TTM treatment group, SAE severity was reported as Grade 1 in 1 participant, Grade 2 in 4 participants, no Grade 3 events, Grade 4 in 1 participant (same participant also experienced a Grade 2 SAE), and Grade 5 in 1 participant with an SAE of “Hepatic encephalopathy.” All SAEs in both treatment groups were assessed as unrelated.

Most common SAEs system organ class for TTM were nervous system disorders (n=6 [4.4%]) and SoC were gastrointestinal disorders and musculoskeletal and connective tissue disorders (n=2 [2.9%] each).

### Psychiatric Disorders (SOC)

Neurologic AESI in the SOC “Psychiatric disorders” were only observed in the TTM treatment group. Seven events in 5 (3.6%) participants were reported: depression, enuresis, insomnia, paranoia, sleep disorder, and apathy and irritability (both in 1participant). All except one event were non-serious and low grade (Grade 1 and Grade 2). Of the 7 events, only 2 resulted in dose modification (1 dose reduction and 1 dose increase). The outcome for 3 events was “resolved” and for 4 events was “ongoing.” Only 1 SAE was reported (“Paranoia aggravated”). No Grade 4 or 5 events were reported, and no events led to study drug discontinuation.

### Other Significant Events and Laboratory Findings

### Liver Effects (Elevations in Liver Transaminase Levels)

Overall, hepatic enzyme elevation (ALT, aspartate aminotransferase [AST], and/or GGT) AEs were reported in a higher percentage of participants treated with TTM compared with SoC in Study 301 (see Table 19). The most commonly reported event for TTM groups was ALT increased (14.6% vs 2.9% for SoC), and, in the TTM groups, accounted for 4.3% (25/577) of treatment-emergent adverse events (TEAEs). These events typically occurred in the first 4-12 weeks and were generally mild to moderate in severity, asymptomatic, reversible, and normalized with dose adjustments and/or interruptions. Hepatic enzyme elevation AEs are summarized in Table 19.

Based on laboratory data, 29 participants treated with TTM had post-baseline elevation of ALT of ≥3× upper limit of normal (ULN):
14 (10.2%) participants had ALT>3×ULN but≤5×ULN
10 (7.3%) participants had ALT>5×ULN but≤10×ULN
1 (0.7%) participants had ALT>10×ULN but≤20×ULN
4 (2.9%) participants had elevated ALT>20×ULN

In participants treated with SoC, 6 had post-baseline elevation of ALT≥3×ULN: 3 (4.3%) participants each in the ranges ALT>3×ULN but≤5×ULN and ALT>5×ULN but≤10×ULN.

One TTM-treated participant experienced ALT>3×ULN and concurrently total bilirubin>2×ULN. The participant's underlying hepatic disease was a confounding factor and this case was adjudicated as unlikely related to TTM by an independent hepatic adjudication panel. Treatment with TTM was re-initiated and liver tests remained normal.

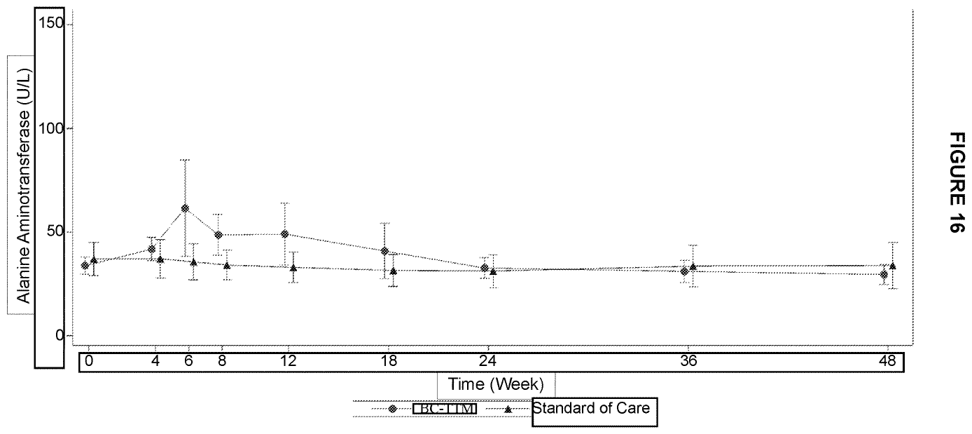

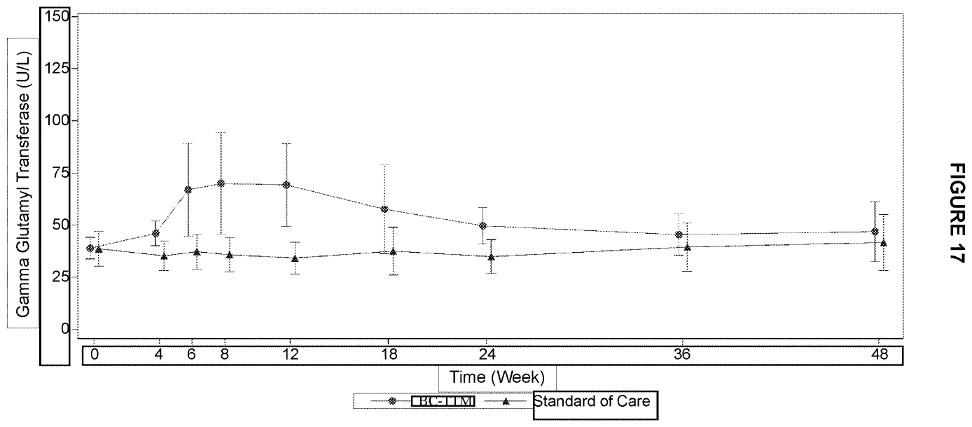

Mean and 95% Cl for ALT and GGT values over time are depicted in FIG. 16 and FIG. 17, respectively. In the TTM group, ALT levels increased from baseline and peaked at week 6, then trended to decrease towards baseline by week 24. GGT levels increased from baseline until week 6, stabilized until week 12, then trended towards baseline until week 36 in the TTM group. Overall, in the SoC group, no clinically significant changes from baseline in ALT and GGT were observed.

TABLE 19
Hepatic Enzyme Elevation Adverse Events
(Primary Evaluation Period - Safety Set)

| | | |
|---|---|---|
| MedDRA | TTM, N = 137 | SoC, N = 70 |

| Preferred Term | N (%) | E | N (%) | E |
|---|---|---|---|---|
| ALT increased | 20 (14.6) | 25 | 2 (2.9) | 2 |
| AST increased | 4 (2.9) | 4 | 0 | 0 |
| GGT increased | 3 (2.2) | 3 | 0 | 0 |
| Hepatic enzyme | 3 (2.2) | 5 | 0 | 0 |
| increased | | | | |
| LFT increased | 4 (2.9) | 4 | 0 | 0 |
| LFT abnormal | 2 (1.5) | 2 | 0 | 0 |
| Hepatic function | 3 (2.2) | 3 | 0 | 0 |
| abnormal | | | | |
| Hypertransaminasemia | 2 (1.5) | 4 | 0 | 0 |
| Liver disorder | 1 (0.7) | 1 | 1 (1.4) | 1 |
| Hyperbilirubinemia | 1 (0.7) | 1 | 0 | 0 |
| Jaundice | 1 (0.7) | 1 | 0 | 0 |

Abbreviations: ALT = alanine transaminase; AST = aspartate aminotransferase; E = events; GGT = gamma-glutamyl transferase; LFT = liver function tests; MedDRA = Medical Dictionary for Regulatory Activities; N = number of participants; SoC = standard of care.

Dyslipidemia: Routine lipid monitoring was not originally included in the clinical studies. Through medical monitoring and review of available local laboratory data, it was identified that three 301 participants experienced liver enzyme elevations with concurrent elevations in cholesterol and TG. Review of data from Study 201 showed that 7 participants experienced total cholesterol increase above the ULN and all 7 had concurrent ALT elevations. Consequently, routine lipid monitoring was added to Study 301 and retrospective analysis was performed on all participants using available retained samples.

The retrospective analysis identified an imbalance of lipid results at baseline between the TTM and SoC groups. Elevated cholesterol at baseline was found in 19% of those on TTM and 10% of those on SoC; 13 (9.5%) of those on TTM and 5 (7.1%) of those on SoC had decreased high density lipoprotein (HDL); 10.9% of those on TTM and 7.1% of those on SoC had elevated low density lipoprotein (LDL). The proportion with elevated TG was similar in both groups at baseline (16.8% of those on TTM and 17.1% of those on SoC.)

Shift analysis from baseline to worst value during study for cholesterol and TG was performed. The results showed a higher percentage of participants in the TTM group in comparison with SoC experienced Grade 1 (43.8% vs 32.9%), Grade 2 (5.1% vs 0), and Grade 3 (3.6% vs 0) cholesterol elevations. Worst values during study were higher in the TTM group compared with the SoC group for Grade 2 (12.4% vs 2.9%), Grade 3 (5.1% vs 1.4%), and Grade 4 (2.9% vs 0) TG. Shift from baseline to worst values during study for cholesterol and triglycerides are presented in Table 20.

TABLE 20
Summary of Lipid Shifts from Baseline by CTCAE Toxicity
Grade (Primary Evaluation Period - Safety Set)

| | Grade 0 | | Grade 1 | | Grade 2 | | Grade 3 | | Grade 4 | |
|---|---|---|---|---|---|---|---|---|---|---|
| Primary-Worst | N (%) | | N (%) | | N (%) | | N (%) | | N (%) | |
| Highest Post- | TTM | SoC | TTM | SoC | TTM | SoC | TTM | SoC | TTM | SoC |
| Baseline | N = 137 | N = 70 | N = 137 | N = 70 | N = 137 | N = 70 | N = 137 | N = 70 | N = 137 | N = 70 |
| Cholesterol | 65 | 47 | 60 | 23 | 7 | 0 | 5 | 0 | 0 | 0 |
| | (47.4) | (67.1) | (43.8) | (32.9) | (5.1) | | (3.6) | | | |
| Triglycerides | 63 | 32 | 46 | 35 | 17 | 2 | 7 | 1 | 4 | 0 |
| | (46.0) | (45.7) | (33.6) | (50.0) | (12.4) | (2.9) | (5.1) | (1.4) | (2.9) | |

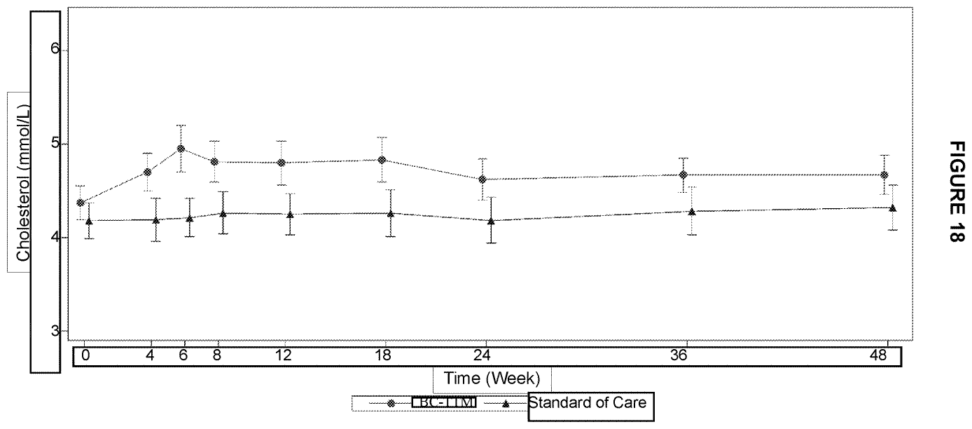

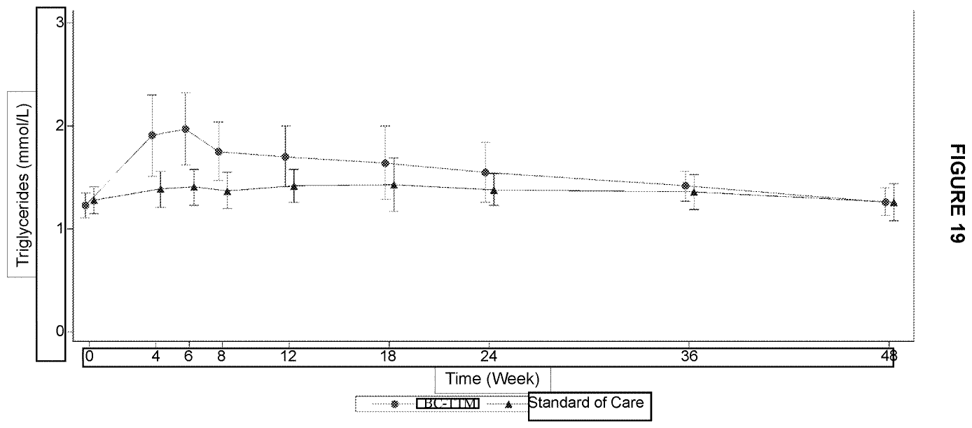

Mean and 95% C in total cholesterol over time are depicted in FIG. 18 where elevations over baseline are seen throughout the primary study analysis period in the TTM group but not the SoC group. In the TTM group, cholesterol levels increased from baseline and peaked around week 6, then trended to decrease towards baseline until week 24 and ultimately stabilizing at slightly higher levels than baseline. In FIG. 19, changes in TG were mainly limited to the early part of the treatment period with an increase from baseline until week 6, then gradual return to baseline by week 36.

Analysis of the lipid profile was conducted retrospectively, using frozen plasma biomarker specimens collected at scheduled study visits. Events under the Cardiac disorder SOC were reviewed, with identification of only one AE potentially correlated with dyslipidemia.

Events under the Gastrointestinal disorders SOC were also reviewed, with no events (e.g., pancreatitis) identified as potentially correlated with dyslipidemia. As described above, lipid abnormalities and liver enzyme elevations have been reported during the primary analysis period of Study 301, and these elevations were more frequent in the TTM group than SoC. Generally, the lipid abnormalities were asymptomatic, transient, and not associated with any significant clinical consequences.

Cytopenia: Copper is an essential micronutrient involved in the catalytic function of several key enzymes involved in various processes throughout the body, including processes in bone marrow and central nervous system. Acquired or inherited copper deficiency may manifest in multiple organ systems but hematologic abnormalities were the most common. Copper deficiency manifests with anemia, neutropenia, and less frequently also with thrombocytopenia.

Hematologic AEs were observed in the clinical program for TTM and are presented in Table 21. The majority of the hematologic events were non-serious, low grade, and resolved with dose modifications. Treatment with TTM was discontinued due to an AE of neutropenia (Grade 2) and anemia (Grade 1) in 1 participant each. Overall, the incidence of hematologic AEs was similar among the TTM and SoC treatment groups.

TABLE 21
Summary of Hematological Adverse Events
(Primary Evaluation Period - Safety Set)

| | Overall | |
|---|---|---|
| MedDRA | TTM, N = 137 | SoC, N = 70 |

| Preferred Term | N (%) | E | N (%) | E |
|---|---|---|---|---|
| Anemia | 6 (4.4) | 7 | 2 (2.9) | 2 |
| Neutropenia | 6 (4.4) | 6 | 2 (2.9) | 2 |
| Neutrophil count | 2 (1.5) | 4 | 0 | 0 |
| decreased | | | | |
| Leukopenia | 2 (1.5) | 3 | 3 (4.3) | 3 |
| Thrombocytopenia | 1 (0.7) | 2 | 2 (2.9) | 2 |

At baseline, decreased neutrophil levels (0.7% vs 1.4%) were similar between TTM and the SoC group, decreased platelet levels (21.9% vs 28.6%) were higher in the SoC group compared to TTM group, and decreased hemoglobin levels (17.5% vs 14.3%) were slightly higher in TTM group compared with SoC group.

Shift from baseline to worst values during study for neutrophils, platelets, and hemoglobin are presented in Table 22. Overall, the results showed a similar percentage in shift from baseline to worst values for neutrophils, platelets, and hemoglobin; except for Grade 1 hemoglobin (41.6% vs 24.3%), Grade 1 platelets (24.1% vs 17.1%), and Grade 3 neutrophils (6.6% vs 2.9%) which were higher in TTM group than the SoC group.

TABLE 22
Summary of Hematologic Shifts from Baseline by CTCAE Toxicity
Grade (Primary Evaluation Period - Safety Set)

| | Grade 0 | | Grade 1 | | Grade 2 | | Grade 3 | | Grade 4 | |
|---|---|---|---|---|---|---|---|---|---|---|
| Primary-Worst | N (%) | | N (%) | | N (%) | | N (%) | | N (%) | |
| Highest Post- | TTM | SoC | TTM | SoC | TTM | SoC | TTM | SoC | TTM | SoC |
| Baseline | N = 137 | N = 70 | N = 137 | N = 70 | N = 137 | N = 70 | N = 137 | N = 70 | N = 137 | N = 70 |
| Neutrophils | 107 | 57 | 0 ^{a} | 0 ^{a} | 19 | 10 | 9 | 2 | 2 | 1 |
| | (78.1) | (81.4) | | | (13.9) | (14.3) | (6.6) | (2.9) | (1.5) | (1.4) |
| Platelets | 90 | 46 | 33 | 12 | 8 | 6 | 5 | 4 | 1 | 2 |
| | (65.7) | (65.7) | (24.1) | (17.1) | (5.8) | (8.6) | (3.6) | (5.7) | (0.7) | (2.9) |
| Hemoglobin | 74 | 50 | 57 | 17 | 5 | 3 | 1 | 0 | 0 | 0 |
| | (54.0) | (71.4) | (41.6) | (24.3) | (3.6) | (4.3) | (0.7) | | | |

^{a} The central laboratory's lower limit of normal for neutrophils is 1000 cells/μL. The CTCAE definition of Grade 1 neutrophils reduced is <LLN to 1500 cells/μL. Based on LLN = 1000 cells/μL, there is no possible result which meets the CTCAE definition for Grade 1 neutrophils reduced. Any neutrophil result less than 1000 cells/μL is reported as Grade 3 or Grade 4.

Safety Findings in Participants Who Switched from SoC to TTM in the Extension Period

An analysis was performed on preliminary data to compare AEs from Study 301 participants receiving SoC during the Primary Evaluation Period (48 weeks) and the subset of those participants who switched to TTM during the Extension Period. The analysis was performed to identify imbalances in AE reporting after the switch from SoC to TTM and compare the results to the imbalances observed in events by SoC during the Primary Evaluation Period [TTM vs SoC].

During the Primary Evaluation Period in Study 301, disproportionate SoCs (>5% difference) between TTM and SoC included Ear and labyrinth disorders [5.1% vs 0%], General disorders and administration site conditions [21.2% vs 10.0%], Hepatobiliary disorders [6.6% vs 1.4%], Investigations [33.6% vs 2.9%], Nervous system disorders [34.3% vs 21.4%], Psychiatric disorders [19.0% vs 4.3%], and Skin and subcutaneous tissue disorders [21.9% vs 5.7%]. These are presented in Table 23.

Among the participants who switched from SoC to TTM, there were 4 SoCs identified with a higher percentage (defined as >5% difference) of participants experiencing AEs which included the following: General disorders and administration site conditions [10% vs 18%], Investigations [2.9% vs 29.5%], Metabolism and nutrition disorders [4.3% vs 11.5%], and Skin and subcutaneous tissue disorders [5.7% vs 13.1%]. The imbalances observed within the respective SoCs were driven by events of fatigue, ALT elevation, lipid elevations (i.e., hyperlipidemia, hypertriglyceridemia, and dyslipidemia), and pruritus as shown in Table 24.

There was no disproportionality in AE reporting identified from any other SoC (see Table 24-1). Notably, and in contrast to the imbalance observed in the Primary Evaluation Period, the switch analysis did not show an imbalance in the Nervous system disorders or Psychiatric disorders SoCs.

TABLE 23
Disproportionate SOCs (>5%) (Primary
Evaluation Period in Study 301, 48 Weeks)

| | TTM | SoC |
|---|---|---|
| | N = 137 | N = 70 |
| System Organ Class | PY = 118.9 | PY = 62.4 |
| Preferred Term | N (%) | N (%) |
| Ear and labyrinth disorders | 7 (5.1) | 0 |
| General disorders and | 29 (21.2) | 7 (10.0) |
| administration site conditions | | |
| Fatigue | 13 (9.5) | 3 (4.3) |
| Pyrexia | 8 (5.8) | 0 |
| Hepatobiliary disorders | 9 (6.6) | 1 (1.4) |
| Investigations | 46 (33.6) | 2 (2.9) |
| Alanine aminotransferase | 20 (14.6) | 2 (2.9) |
| increased | | |
| Aspartate aminotransferase | 4 (2.9) | 0 |
| increased | | |
| Gamma-glutamyltransferase | 3 (2.2) | 0 |
| increased | | |
| Hepatic enzyme increased | 3 (2.2) | 0 |
| Neutrophil count decreased | 2 (1.5) | 0 |
| Nervous system disorders | 47 (34.3) | 15 (21.4) |
| Psychiatric disorders | 26 (19.0) | 3 (4.3) |
| Skin and subcutaneous | 30 (21.9) | 4 (5.7) |
| tissue disorders | | |
| Pruritus | 5 (3.6) | 0 |
| Rash | 9 (6.6) | 1 (1.4) |

Abbreviations: N = number of participants; PY = patient years; SOC = System Organ Class

TABLE 24
Disproportionate Terms (>5% difference) by SoC and Selected
Preferred Terms in Participants Who Switched from SoC to TTM

| | Weeks 0-48 | | Weeks 49-96 | |
|---|---|---|---|---|
| | on Study SoC | | on Study TTM | |
| | N = 70 | | N = 61 | |
| System Organ Class | PY = 62.4 | | PY = 55.4 | |
| Preferred Term | N (%) | E | N (%) | E |
| General disorders and | 7 (10.0) | 8 | 11 (18.0) | 12 |
| administration site conditions | | | | |
| Fatigue | 3 (4.3) | 3 | 5 (8.2) | 5 |
| Pyrexia | 0 | 0 | 1 (1.6) | 1 |
| Investigations | 2 (2.9) | 2 | 18 (29.5) | 29 |
| Alanine aminotransferase | 2 (2.9) | 2 | 12 (19.7) | 14 |
| increased | | | | |
| Aspartate aminotransferase | 0 | 0 | 4 (6.6) | 4 |
| increased | | | | |
| Gamma-glutamyltransferase | 0 | 0 | 3 (4.9) | 3 |
| increased | | | | |
| Hepatic enzyme increased | 0 | 0 | 3 (4.9) | 3 |
| Neutrophil count decreased | 0 | 0 | 1 (1.6) | 1 |
| Metabolism and nutrition | 3 (4.3) | 4 | 7 (11.5) | 10 |
| disorders | | | | |
| Hyperlipidaemia | 0 | 0 | 1 (1.6) | 1 |
| Hypertriglyceridaemia | 0 | 0 | 1 (1.6) | 3 |
| Dyslipidemia | 0 | 0 | 1 (1.6) | 1 |
| Skin and subcutaneous | 4 (5.7) | 7 | 8 (13.1) | 15 |
| tissue disorders | | | | |
| Pruritus | 0 | 0 | 2 (3.3) | 2 |
| Rash | 1 (1.4) | 1 | 1 (1.6) | 5 |
| Rash pruritic | 0 | 0 | 1 (1.6) | 1 |
| Eczema | 0 | 0 | 1 (1.6) | 1 |
| Hand dermatitis | 0 | 0 | 1 (1.6) | 1 |

TABLE 24-1
Proportionate Terms by SOC and Selected Preferred
Term in Participants Who Switched from SoC to TTM

| | Weeks 0-48 | | Weeks 49-96 | |
|---|---|---|---|---|
| | on Study SoC | | on Study TTM | |
| | N = 70 | | N = 61 | |
| System Organ Class | PY = 62.4 | | PY = 55.4 | |
| Preferred Term | N (%) | E | N (%) | E |
| Blood and lymphatic system | 9 (12.9) | 10 | 7 (11.5) | 18 |
| disorders | | | | |
| Cardiac disorders | 3 (4.3) | 4 | 3 (4.9) | 5 |
| Congenital, familial, | 1 (1.4) | 1 | 0 | 0 |
| and genetic disorders | | | | |
| Ear and labyrinth disorders | 0 | 0 | 1 (1.6) | 1 |
| Eye disorders | 1 (1.4) | 1 | 0 | 0 |
| Gastrointestinal disorders | 23 (32.9) | 39 | 18 (29.5) | 30 |
| Hepatobiliary disorders | 1 (1.4) | 1 | 2 (3.3) | 3 |
| Infections and infestations | 24 (34.3) | 36 | 17 (27.9) | 23 |
| Injury, poisoning and | 6 (8.6) | 8 | 7 (11.5) | 13 |
| procedural complications | | | | |
| Musculoskeletal and | 12 (17.1) | 13 | 7 (11.5) | 7 |
| connective tissue disorders | | | | |
| Neoplasms benign, malignant, | 1 (1.4) | 1 | 1 (1.6) | 1 |
| and unspecified (incl | | | | |
| cysts and polyps) | | | | |
| Nervous system disorders | 15 (21.4) | 32 | 11 (18.0) | 12 |
| Product issues | 1 (1.4) | 1 | 0 | 0 |
| Psychiatric disorders | 3 (4.3) | 3 | 3 (4.9) | 3 |
| Anxiety | 0 | 0 | 1 (1.6) | 1 |
| Sleep disorder | 0 | 0 | 1 (1.6) | 1 |
| Restlessness | 1 (1.4) | 1 | 0 | 0 |
| Affective disorder | 1 (1.4) | 1 | 0 | 0 |
| Attention deficit | 1 (1.4) | 1 | 0 | 0 |
| hyperactivity disorder | | | | |
| Post-traumatic stress disorder | 0 | 0 | 1 (1.6) | 1 |
| Renal and urinary disorders | 2 (2.9) | 2 | 1 (1.6) | 2 |
| Reproductive system | 4 (5.7) | 4 | 0 | 0 |
| and breast disorders | | | | |
| Respiratory, thoracic, | 9 (12.9) | 12 | 3 (4.9) | 4 |
| and mediastinal disorders | | | | |
| Vascular disorders | 3 (4.3) | 3 | 3 (4.9) | 5 |

Overall, most AEs observed in Study 301 were nonserious, mild or moderate, manageable, and did not result in treatment discontinuation. The commonly (>10%) observed AEs included ALT increased and nasopharyngitis. The risks observed in participants treated with TTM including hepatic effects (elevations in liver transaminase levels), dyslipidemia, and cytopenias were generally asymptomatic, reversible with dose modification, and not associated with any clinical consequences.

Consistent with the above, analysis of data in participants who switched from SoC treatment to TTM showed an increased incidence of laboratory abnormalities (increased ALT, increased cholesterol and increased TG, decreased neutrophils), further supporting an association with TTM.

Worsening of neurologic symptoms is a known concern for SoC; this is primarily associated with penicillamine but has also been observed with trientine and zinc. The mechanism of neurologic worsening is unknown but association with unbound copper has been previously hypothesized. The data from the Primary Evaluation Period in Study 301 showed an imbalance in neurologic and psychiatric events with a higher incidence in participants treated with TTM vs SoC; however, these imbalances were not observed in the participants who switched from SoC treatment to TTM during the Extension Period. As Study 301 is an open-label study, there is the potential for biased reporting by participants who know they are receiving the investigational medication. The cited finding suggests a lack of an association between TTM and neurologic and psychiatric AEs.

Overall, TTM has an acceptable safety profile and is generally well-tolerated in participants with WD.

Cirrhosis at Baseline and Albumin Levels During TTM Treatment: Across the 301, 201, and 205 studies, 102 participants had cirrhosis at baseline. Study 205 is registered with ClinicalTrials.gov, number NCT04422431, and incorporated by reference in its entirety. Of the total 102, 13 participants experienced changes in albumin levels (indicative of changes in synthetic liver function) over the course of treatment with TTM. The albumin levels in the other 89 participants remained stable (indicative of stable liver function) during the studies. The changes in albumin levels in these 13 participants are listed in Table 25 below (units shown are mg/dL throughout).

TABLE 25
Albumin Levels in Cirrhotic Participants Who Experienced
Changes in Liver Function During TTM Treatment

| | | |
|---|---|---|
| Time Point | Albumin Level (mg/dL) | Note |

Study 301 (Normal Reference Range: 35-55 mg/dL)

| | | |
|---|---|---|
| Participant 1 | | |
| Baseline | 33 | |
| Week 48 | 47 | |
| Week 96 | 48 | |
| Participant 2* | | |
| Baseline | 37 | |
| Week 48 | 34 | |
| Week 60 | 25 | Early termination |
| | | from study |
| Participant 3 | | |
| Baseline | 34 | |
| Week 48 | 36 | |
| Week 60 | 34 | |
| Participant 4 | | |
| Baseline | 28 | |
| Week 48 | 31 | |
| Participant 5 | | |
| Baseline | 32 | |
| Week 48 | 37 | |
| Week 96 | 37 | |
| Participant 6 | | |
| Baseline | 29 | |
| Week 24 | 35 | |
| Week 48 | 42 | |
| Week 96 | 40 | |
| Participant 7 | | |
| Baseline | 42 | Week 48 Extension |
| | | Day 1 |
| Week 8 | 37 | |
| Week 24 | 36 | |
| Week 30 | 35 | |
| Week 48 | 35 | |
| Week 144 | 37 | |
| Participant 8 | | |
| Baseline | 42 | Week 48 Extension |
| | | Day 1 |
| Week 120 | 32 | Early termination |
| | | from study |

Study 201 (Normal Reference Range: 33-49 mg/dL)

| | | |
|---|---|---|
| Participant 9 | | |
| Baseline | 29 | |
| Week 48 | 45 | |
| Week 150 | 46 | Early termination |
| | | from study |
| Participant 10 | | |
| Baseline | 29 | |
| Week 48 | 40 | |
| Week 98 | 40 | |
| Week 163 | 39 | Early termination |
| | | from study |
| Participant 11 | | |
| Baseline | 31 | |
| Week 48 | 44 | |
| Week 98 | 46 | |
| Week 150 | 44 | |

Study 205 (Normal Reference Range: 35-55 mg/dL)

| | | |
|---|---|---|
| Participant 12 | | |
| Baseline | 36 | |
| Day 43 | 33 | Unscheduled visit |
| Day 57 | 30 | Unscheduled visit |
| Day 169 | 34 | |
| Day 253 | 34 | Unscheduled visit |
| Day 337 | 37 | Early termination |
| | | from study |
| Participant 13* | | |
| Baseline | 45 | |
| Day 345 | 27 | |

*This patient ultimately experienced SAE and underwent liver transplant.

These results demonstrate that out of 102 participants with cirrhosis at baseline, 87% maintained liver function (as measured by albumin levels) during TTM treatment. Of the 13 cirrhotic participants who experienced changes in liver function, 7 participants undergoing TTM treatment experienced improvements such that albumin levels that had fallen outside the normal range returned to the normal range during TTM treatment.

### Example 2: Further Evaluation of Cohort 1—Incomplete and Intolerant Responders

As described above, patients with Wilson disease (WD) who have been treated for years with standard of care (SoC) can remain neurologically symptomatic despite perceived overall disease stability. Existing treatments also have burdensome dosing schedules and/or side effects and are associated with low compliance rates. Efficacy and safety of tiomolibdate choline (BC-TTM; INN: tiomolibdic acid; USAN: tiomolibdate choline; also known as ALXN1840; formerly known as WTX101) was investigated in patients with ongoing neurologic symptoms or a history of intolerance to standard of care (SoC) treatment.

As described in Example 1, in the open-label, Phase 3 FoCus trial (NCT03403205), 214 patients were stratified by duration of prior SoC (cohort 1: >28 days; cohort 2: 0-28 days) and then randomized 2:1 to BC-TTM (15 QOD-60 mg QD) or SoC (penicillamine, trientine and/or zinc) for 48 weeks (W). The Statistical Analysis Plan for Study 301 included a pre-specified analysis subset of study participants who were intolerant or had an incomplete response to prior therapy with penicillamine, trientine, and/or zinc. These groups were more precisely defined as intolerant (identified by prior treatment history or adverse event prior to Day 1 of TTM treatment) or incomplete responders (those with a baseline UWDRS Part II or Part III total score>0). The definition for this analysis subset from Study 301 is referred to as Subpart E herein. For purposes of this disclosure, study participants who had an incomplete response to prior therapy with penicillamine, trientine, and/or zinc may also be referred to as “incomplete responders” or “inadequate responders.”

For analysis, the participant pool was extended to include those from Studies 201 and 205 who also met these criteria (see Table 26). The Subpart E population was limited to those participants with previous treatment>28 days (identified in the studies described herein as Cohort 1) to ensure that participants had a minimum course of another treatment before switching to TTM treatment. Note that Study 201 did not collect data allowing a participant to be declared intolerant to prior treatments. This yielded a Subpart E population of 164 study participants.

TABLE 26
Source of Subpart E Population

| | | Evaluable | | | | |
|---|---|---|---|---|---|---|
| | | Patients | | | | |
| | | with >28 | | | | |
| | | Days Prior | Subpart E Population | | | |
| | Evaluable | Therapy at | | Met Only | | Met at |
| | Patients | Initiation | Met Only | Incomplete | Met | least |
| | Treated | of TTM | Intolerant | Response | Both | One |
| Study | with TTM | Treatment | Criterion | Criterion | Criteria | Criterion |
| 201 | 28 | 10 | 0 | 10 | 0 | 10 |
| 205 | 30 | 30 | 0 | 20 | 3 | 23 |
| 301 | | | | | | |
| Started TTM | 137 | 104 | 6 | 68 | 14 | 88 |
| in PEP | | | | | | |
| Started TTM | 61 | 61* | 0 | 29 | 14 | 43 |
| after PEP | | | | | | |

Abbreviations: PEP = primary evaluation period
*includes participants who had <28 days of treatment at Study 301 initiation, as they were treatment-experienced when switched from SoC to TTM

Endpoints (directly measured non-ceruloplasmin-bound copper [dNCC], UWDRS III (rater blinded) total and individual item scores, Clinical Global Impression-Improvement [CGI-I]) were evaluated at baseline (BL) and Week 48 (W48). Normalization (score decrease to 0), improvement (≥1 point decrease), stability (no change), or worsening (≥1 point increase) of total and individual UWDRS III item scores were evaluated at W48.

### Justification of Subpart E Population

Patients with WD who experience an incomplete or intolerant response to standard of care treatment have a high unmet medical need. Those patients with an incomplete response to current therapy have persistent symptoms despite treatment. Scientific literature reports hepatic worsening in patients on SoC treatment over a follow-up>10 years: 5-24% of pre-symptomatic patients develop hepatic symptoms that in some cases progress to hepatic failure (Merle et al., 2007, Gut, 56: 115-120, Dziezyc et al., 2014, Eur J Neurol, 21(2): 332-337). A long-term follow-up study showed that hepatic treatment failure was more frequently observed in patients taking zinc (14/88 treatments) than in patients taking a metal chelator (4/313 treatments) (Weiss et al., 2011, Gastroenterology, 140(4): 1189-198). Literature reports that worsening of neurologic symptoms in patients with neurologic symptoms at baseline occurred in 5% to 7% of patients treated with penicillamine and 16% to 20% of patients treated with trientine, depending on whether treatment was first or second line, after 6 to 48 months of treatment (Weiss et al., 2013, Clin Gastroenterol Hepatol., 11(8):1028-1035). Also, the literature reports neurologic disease progression being observed in long-term studies (>10 years). For example, in patients with neurologic symptoms at diagnosis who were followed for a mean of 16.7 years, neurological worsening occurred in 24% of treated patients (14/58) (Merle et al., 2007, Gut, 56: 115-120). And in pre-symptomatic patients who were treated and followed for over >10 years, neuropsychiatric symptoms developed in 19% of patients (Merle et al., 2007, Gut, 56: 115-120, Dziezyc et al., 2014, Eur J Neurol, 21(2): 332-337).

In Study 301, the participants previously treated for >28 days with an intolerance or incomplete response to prior therapy presented with persistent symptoms or organ damage at baseline: 100% of participants had neurologic deficits (UWDRS part II>0 and/or UWDRS part III>0, as per the definition of incomplete response), 79% of participants had psychiatric symptoms (BPRS>24), and 40% of the participants had compensated cirrhosis. This shows a high burden of disease despite previous treatment for a mean duration of 11.2 years.

Patients with intolerance to SoC might require a change of treatment, and additional treatment options would be beneficial. Medical literature suggests that penicillamine may be less well-tolerated than other first-line treatment options for WD, as it can be associated with severe side effects requiring drug withdrawal in approximately 30% of patients (Schilsky et al., 2022, Hepatology, 00: 1-49). Safety concerns requiring immediate discontinuation of penicillamine include early sensitivity reactions which may occur during the first 1-3 weeks, later reactions including nephrotoxicity, bone marrow toxicity including severe thrombocytopenia or total aplasia that may be irreversible despite discontinuation of therapy (Schilsky et al., 2022, Hepatology, 00: 1-49). Dermatological toxicities may include progeric changes in the skin and elastosis perforans serpingosa, pemphigous or pemphigoid lesions, lichen planus, and aphthous stomatitis (Schilsky et al., 2022, Hepatology, 00: 1-49). Trientine has few side effects; colitis can develop in rare cases (Schilsky et al., 2022, Hepatology, 00: 1-49). Zinc has very few side effects; gastric irritation or gastritis is the most common adverse effect occurring in ~⅓ of patients, which may be dependent on the type of zinc salt (Schilsky et al., 2022, Hepatology. 00: 1-49).

An unmet need, for example, in patients with incomplete or intolerant response to current treatments is adherence and persistence to therapy. With dosing up to 3 times daily for the current SoC, real-world adherence to SoC treatment in WD may be problematic in approximately a third of patients (Jacquelet et al., 2021, J Inherit Metab Dis., 44(6):1481-1488, Maselbas et al., 2019, BMC Neurol. 19(278): 1-6). Patients non-persistent to therapy presented more often with worsening WD than persistent patients (52.3% vs. 2.4%) (Maselbas et al., 2019, BMC Neurol. 19(278): 1-6). Indeed, in Study 301, compliance with SoC was over 90%, in contrast with the real-world evidence referenced here.

### Analysis Subpopulation

The efficacy of TTM in Wilson disease has been evaluated in a population of treatment-experienced patients who had an incomplete response to penicillamine, trientine or zinc (as determined by residual neurologic signs and symptoms) or who were intolerant to these treatments. These patients were drawn from: 1) one single arm Phase 2 study (Study 201), which contributed 10 adult patients; 2) one single arm Phase 2 study (Study 205), which contributed 23 adult patients; and 3) one Phase 3 study (Study 301), which contributed 131 patients (adults and adolescents aged 12-17 years). Study 301 also allows for comparison of treatment with TTM to treatment with then-available therapies (the chelators penicillamine or trientine, with or without zinc; or zinc alone).

Single-Arm Study of TTM in Patients with Wilson Disease

Study 201, a single-arm Phase 2 study, assessed the effect of BC-TTM over 24 weeks in adult patients with Wilson disease. Individualized dosing was allowed, and the median dose administered to the 10 patients meeting the analysis definition was 18.7 mg/day.

Single Arm, Liver Biopsy Study of TTM in Patients with Wilson Disease

Study 205, a single-arm Phase 2 study, assessed the impact of treatment with tiomolibdate choline on the liver during a 48-week treatment period in adult patients with Wilson disease. The biopsies were taken at baseline and at week 48 and assessed for change in liver copper concentration. The median dose administered to the 23 patients meeting the analysis definition was 16.5 mg/day.

Comparison of TTM to SoC in Patients with Wilson Disease

Study 301, a randomized, active-control study, compared the efficacy of TTM with that of chelators (penicillamine or trientine, with or without zinc) or zinc alone in adult and adolescent patients with Wilson disease. The endpoint was copper mobilization, which was measured as daily mean area under the effect-time curve (AUEC) for plasma total concentration from time 0 to Week 48 (AUEC_{0-48 weeks}). TTM was superior to the control arm receiving standard of care (SoC) in mobilizing copper.

### Clinical Findings

Clinical benefit for the analysis subpopulation is supported by:
Superiority of TTM treatment over treatment with SoC in mobilizing copper from tissues, demonstrated by the efficacy endpoint, dNCC AUEC_{0-48 wks} [μmol/L](p value<0.0001 for Subpart E total population);
Rapid and clinically meaningful within group improvements from baseline in UWDRS Part II scores (patient/care giver reported), seen as early as 24 weeks and continuing over 48 weeks;
Rapid and clinically meaningful within group improvements from baseline in UWDRS Part III scores (blinded neurological examination), seen as early as 24 weeks and continuing over 48 weeks;
Significant between group (TTM vs. SoCs) improvements in CGI-I at Week 48; odds ratio of 4.9 for TTM-treated participants vs SoC-treated participants (p=0.0004); significant between group improvements at Week 48 in mean CGI-I; and
Significant between group (TTM vs. SoCs) improvements of at least 1 point in CGI-S at Week 48; odds ratio of 3.6 for TTM-treated participants vs SoC-treated participants; meaningful within group improvements at Week 48 in mean CGI-S.
   Long-term clinical benefit of TTM is demonstrated by the following data pooled from the 301, 201, and 205 studies:
Clinically meaningful within group long-term improvements from baseline in UWDRS Part II scores (patient/care giver reported) and in UWDRS Part III scores (blinded neurological examination) for more than 48 weeks and up to 5 years.

Long-term treatment data (up to 240 weeks) that are available for these results indicate that both participant-reported and clinical/neurologist-reported scores showed improvement over 240 weeks of TTM treatment.

As described above, the key secondary endpoints of Study 301, meant to provide evidence of direct clinical benefit, were:
Change from baseline in the UWDRS Part II total score (activities of daily living);
Change from baseline in the UWDRS Part III Functional Subscale;
Change from baseline in the UWDRS Part III individual functional items: arising from a chair, gait, speech, and handwriting.
   The key secondary efficacy endpoints were statistically significantly different between TTM and SoC, within the subpopulation that has an incomplete response or is intolerant to SoC. As demonstrated in Table 27, the patients of Subpart E who received TTM treatment had improved UWDRS Part III total scores compared to the baseline value with a 95% confidence interval that did not cross 0, indicating that the p-value was significant. Further, these changes from the baseline were greater than those of patients who received the standard of care treatment. And indeed, the standard of care 95% confidence interval crossed 0, indicating that the changes observed in the UWDRS Part III of these patients were not statistically significant.

TABLE 27
UWDRS Part III: Incomplete or Intolerant
Responders (Covariance Structure - UN)

| | Subpopulation E | | |
|---|---|---|---|
| Visit Statistic | | TTM | SoC |

| | | |
|---|---|---|
| No. patients | 88 | 45 |

Week 48, Change from Baseline

| | | |
|---|---|---|
| N | 79 | 38 |

| | | | | |
|---|---|---|---|---|
| Mean (SD) | −2.64 | (7.921) | −2.10 | (6.959) |
| Median (IQR) | −1.00 | (6.00) | −2.00 | (4.00) |
| Q1, Q3 | −6.00, | 0.00 | −4.00, | 0.00 |
| Min, Max | −28.0, | 31.0 | −22.0, | 11.0 |
| 95% CI | (−4.42, | −0.87) | (−4.39, | 0.18) |
| LSM (SE) | −2.52 | (0.826) | −1.99 | (1.162) |
| 95% CI | (−4.15, | −0.88) | (−4.28, | 0.31) |
| LSM Diff (SE) | −0.53 | (1.426) | | |
| 95% CI | (−3.36, | 2.29) | | |

Abbreviations: SD = standard deviation; IQR = inter-quartile range; Q1, Q3 = quartile 1, quartile 3
LSM and its 95% CI and 2-sided p-value are calculated using an REML based MMRM with fixed effects for baseline, cohort, visit, and cohort-by-visit interaction. For cohort analysis, analysis was performed on each cohort removing cohort and cohort-by-visit interaction. The Kenward-Rodger approximation is used to estimate denominator degrees of freedom. CI's and MMRM estimates are only displayed when n ≥ 3.

Similarly, Table 28 shows that net promoter scores for the UWDRS Part III of the patients who received TTM treatment were greater than those that received standard of care treatment. To calculate net promoter scores, minimal clinically important differences (MCID) were first defined. There is no current definition of MCID for patients with neurological manifestations of WD; although the literature has defined worsening as an increase in 4 points (Czlonkowska et al., 2014, European Journal of Neurology, 21:599-606) or 20% (Poujois et al., 2020, Neurology, 94(21):e2189-e2202) on the UWDRS Part III.

Therefore, distribution-based methods were used to compute MCID threshold, half standard deviation (SD), and standard error of measurement (SEM) for the full Study 301 population and the Subpart E population. The proportion of patients who demonstrated improvement in UWDRS Part III score greater than the MCID threshold and the proportion of patients who demonstrated worsening in UWDRS Part III score greater than the MCID threshold were then calculated. The difference between those two proportions represented the “net promoter score.”

Applying distribution-based methods for the overall cohort, the half SD and SEM MCID values were 6.611 and 5.032, respectively. The incomplete and/or intolerant subgroup analysis led to lower MCIDs of 6.371 half SD and 4.849 SEM. A provisional MCID and responder definition for the UWDRS Part III in WD was approximately 5-6 points of improvement in the overall population and 4-6 points in the incomplete and intolerant subgroup. Applying a 5-point change in UWDRS Part III as the responder threshold to Study 301 data, 44.8% (n=30) of patients in the TTM-treated group versus 29.4% (n=5) patients in the SoC-treated group improved by ≥5 points by 48 weeks, with near similar results observed with a 4-point change threshold for the incomplete and/or intolerant subgroup: 43.1% (n=22) patients on TTM treatment versus 29.4% (n=5) patients on SoC treatment.

TABLE 28
UWDRS Part III: Minimal Clinically Important
Differences (MCID) and Net Promoter Score

| | | | Net Promoter |
|---|---|---|---|
| | Improving | Worsening | Score |
| SD MCID Achievers | | | |
| TTM | 22% | 6% | 16% |
| SoC | 16% | 13% | 3% |
| SE MCID Achievers | | | |
| TTM | 28% | 8% | 20% |
| SoC | 21% | 18% | 5% |

Abbreviations: SD = standard deviation; SE = standard error; MCID = minimal clinically important difference

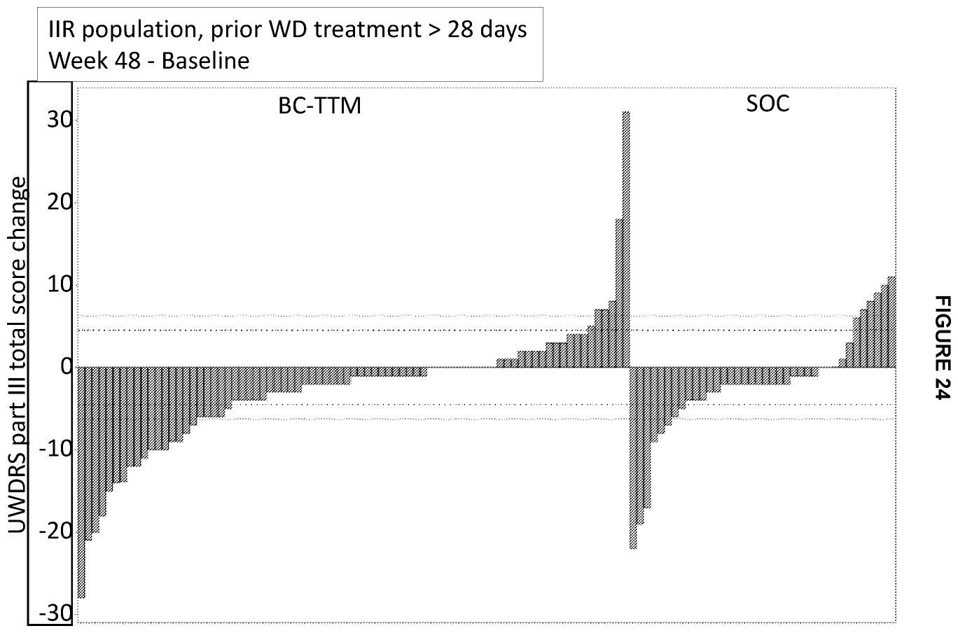

Thus, within the incomplete and intolerant responders, more patients improved their UWDRS part III total score with treatment of TTM than with the SoC. This is further illustrated in FIG. 24, in which each line represents one patient (negative=improvement from baseline; positive=worsening from baseline; blank=no change).

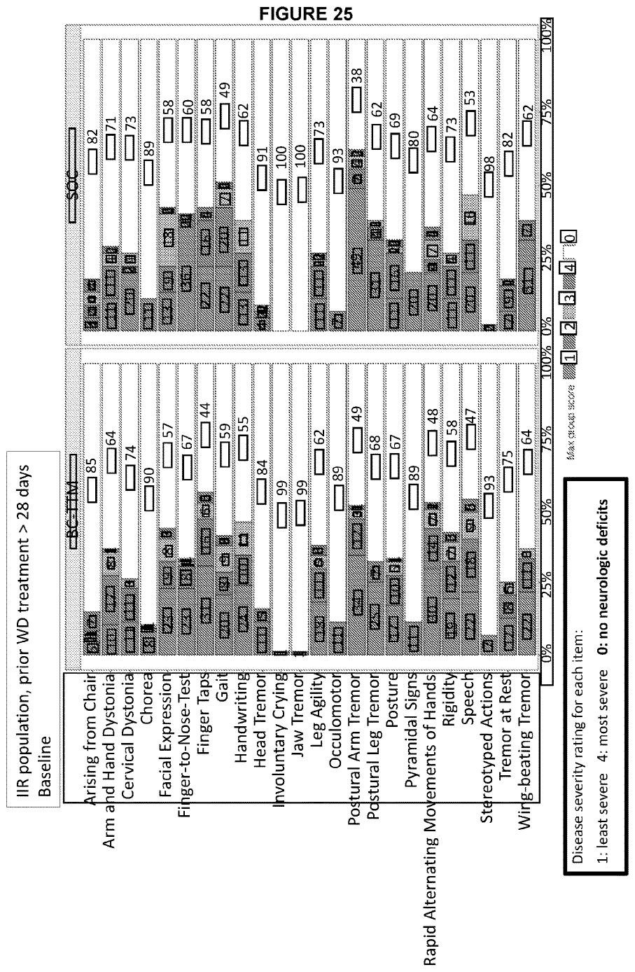

As described above, when looking at the whole population, the key secondary efficacy endpoints were not statistically significantly different between TTM and SoC treatment groups, as demonstrated in Table 14 in Example 1, although there were trends toward stability/improvement for both treatment groups. A possible explanation for the relative lack of improvement in neurology scores is the good baseline status of the majority of patients, as demonstrated by data in Table 15 in Example 1. But upon a closer look at the baseline measurements of neurologic deficits (UWDRS-Ill) for the subgroup of patients with incomplete responses or intolerance to SoC treatment prior to W treatment, it was found that there is a large heterogeneity of symptoms at baseline (“BL”). Wilson Disease symptom heterogeneity is demonstrated in FIG. 25 by showing the distribution of symptoms experienced by patients. As shown in FIG. 25, WD heterogeneity in the trial population was seen in the severity of disease across the treatment groups.

Due to this heterogeneity, the effect of the TTM treatment on individual symptoms of the UWDRS parameters were evaluated. Table 29 reports these results.

TABLE 29
Changes in Individual Symptoms of UWDRS parameters

| | TTM | | | SoC | | | Difference |
|---|---|---|---|---|---|---|---|
| | | Wk | Change | | Wk | Change | (TTM − |
| Sign/Symptom | BL | 48 | (Wk 48 - BL) | BL | 48 | (Wk 48 - BL) | SoC) |
| Arising from chair | 85 | 86 | 1 | 82 | 79 | −3 | 4 |
| Arm and hand Dystonia | 64 | 67 | 3 | 71 | 71 | 0 | 3 |
| Cervical Dystonia | 74 | 82 | 8 | 73 | 76 | 3 | 5 |
| Chorea | 90 | 91 | 1 | 89 | 84 | −5 | 6 |
| Facial Expression | 57 | 58 | 1 | 58 | 63 | 5 | 4 |
| Finger to Nose | 67 | 76 | 9 | 60 | 63 | 3 | 6 |
| Finger Taps | 44 | 59 | 15 | 58 | 53 | 5 | 20 |
| Gait | 59 | 63 | 4 | 49 | 55 | 6 | 2 |
| Handwriting | 55 | 71 | 16 | 62 | 65 | 3 | 13 |
| Head Tremor | 84 | 89 | 5 | 91 | 87 | −4 | 9 |
| Involuntary Crying | 99 | 99 | 0 | 100 | 97 | −3 | 3 |
| Jaw Tremor | 99 | 99 | 0 | 100 | 100 | 0 | 0 |
| Leg Ability | 62 | 66 | 4 | 73 | 66 | −7 | 11 |
| Oculomotor | 89 | 87 | −2 | 93 | 82 | −11 | 9 |
| Postural Arm Tremor | 49 | 58 | 9 | 38 | 55 | 17 | 8 |
| Postural Leg Tremor | 68 | 73 | 5 | 62 | 76 | 14 | 9 |
| Posture | 67 | 71 | 4 | 69 | 74 | 5 | 1 |
| Pyramidal Signs | 89 | 87 | −2 | 80 | 82 | 2 | 4 |
| RAM of Hands | 48 | 62 | 14 | 64 | 47 | −17 | 31 |
| Rigidity | 58 | 72 | 14 | 73 | 82 | 9 | 5 |
| Speech | 44 | 51 | 7 | 51 | 58 | 7 | 0 |
| Stereotyped Actions | 93 | 89 | −4 | 98 | 95 | −3 | 1 |
| Tremor at rest | 75 | 76 | 1 | 82 | 79 | −3 | 4 |
| Wing-beating tremor | 64 | 67 | 3 | 62 | 74 | 12 | 9 |

Table 29 demonstrates that specific symptoms felt by patients were improved over those on the standard of care.

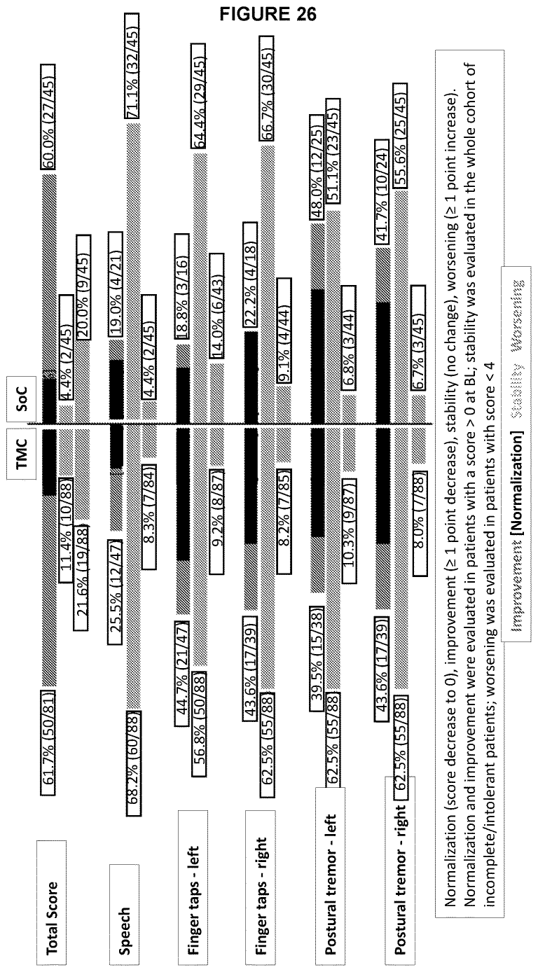

This is further illustrated in FIG. 26. Among the 160 treated patients in cohort 1, 133 (TTM: 88, SoC: 45) had an incomplete response or were intolerant to prior SoC, with 6 patients being intolerant only. At BL, cumulative mean (SD) duration of prior SoC treatment was 149.0 (137.3) and 153.7 (161.9) months in the TTM and SoC groups, respectively. At BL, a score>0 in speech (n=68), finger taps left (n=63), postural tremor right (n=63) and left (n=63) were reported most frequently. Least-squares mean (LSM) difference (standard error [SE]) for TTM versus SoC from W0-W48 was 1.64 (0.297), p<0.0001 for dNCC area under the effect curve; −0.53 (1.426) p=0.7084 for UWDRS III total; and −0.4 (0.15), p=0.0051 for CGI-I. Within-group changes from baseline (LSM [SE]) were −2.52 (0.826), 95% confidence interval (CI) (−4.15, −0.88) for UWDRS III total for TTM-treated patients and −1.99 (1.162), 95% Cl (−4.28, 0.31) for SoC-treated patients. The proportion of patients showing normalization, improvement, stability or worsening on UWDRS III total and most frequently reported individual items at W48 are shown in FIG. 26. The most common treatment-emergent AE that occurred with TTM was alanine aminotransferase elevation. Neurologic AEs were observed in 31.8% and 22.2% of patients in the TTM and SoC groups, respectively and were predominantly nonserious, mild or moderate, and resolved without dose modification. Withdrawals due to an AE occurred in the TTM (n=5) and SOC (n=1) groups.

Overall, in cohort 1 patients with an incomplete response or intolerance to SoC, treatment with TTM or SoC for 48 weeks was associated with disease stability and improvement in neurologic symptoms. Treatment with TTM was generally safe and well tolerated. Further, patients that were considered stable on standard of care but still had long term symptoms were also improving.

Additionally, rapid, clinically meaningful improvements of the UWDRS Part III score were observed following TTM treatment within this sub-group in UWDRS-Ill scores at week 24 and week 48. Table 30 shows these results.

TABLE 30
UWDRS-III Improvements at Week 24 and 48 for Subpopulation E
Cohort: Cohort 1 (Covariance Structure - UN)

| | TTM | | | | SoC |
|---|---|---|---|---|---|
| Visit | Study 301 | Study 201 | Study 205 | Total | Study 301 |
| Statistic | N = 104 | N = 10 | N = 30 | N = 144 | N = 56 |

Week 24, Change from Baseline

| n | 90 | | 8 | | 0 | 98 | | 47 | |
|---|---|---|---|---|---|---|---|---|---|
| Mean (SD) | −0.86 | (5.520) | −13.82 | (11.576) | | −1.92 | (7.096) | −0.79 | (7.611) |
| Median (IQR) | 0.00 | (4.00) | −13.50 | (14.80) | | −1.00 | (5.00) | −1.00 | (4.60) |
| Q1, Q3 | −3.00, | 1.00 | −19.80, | −5.00 | | −4.00, | 1.00 | −2.60, | 2.00 |
| Min, Max | −18.7, | 13.0 | −35.0, | 1.0 | | −35.0, | 13.0 | −26.0, | 23.0 |
| 95% CI | (−2.02, | 0.30) | (−23.50, | −4.15) | | (−3.34, | −0.50) | (−3.03, | 1.44) |
| LSM (SE) | | | | | | −1.72 | (0.648) | −0.71 | (0.963) |
| 95% CI | | | | | | (−3.00, | −0.44) | (−2.61, | 1.19) |
| LSM Diff (SE) | | | | | | −1.00 | (1.161) | | |
| 95% CI | | | | | | (−3.30, | 1.29) | | |

| | |
|---|---|
| P-value | 0.3884 |
| (TTM vs. SoC) | |

Week 48, Change from Baseline

| n | 91 | | 9 | | 27 | | 127 | | 49 | |
|---|---|---|---|---|---|---|---|---|---|---|
| Mean (SD) | −2.24 | (7.458) | −19.44 | (17.889) | −2.81 | (4.820) | −3.58 | (9.178) | −1.59 | (6.188) |
| Median (IQR) | −1.00 | (4.00) | −13.00 | (20.00) | −2.00 | (6.00) | −1.00 | (6.00) | −1.00 | (3.00) |
| Q1, Q3 | −4.00, | 0.00 | −29.00, | −9.00 | −6.00, | 0.00 | −6.00, | 0.00 | −3.00, | 0.00 |
| Min, Max | −28.0, | 31.0 | −48.0, | 2.0 | −17.0, | 6.0 | −48.0, | 31.0 | −22.0, | 11.0 |
| 95% CI | (−3.79, | −0.69) | (−33.20, | −5.69) | (−4.72, | −0.91) | (−5.19, | −1.97) | (−3.37, | 0.19) |
| LSM (SE) | | | | | | | −3.62 | (0.685) | −1.65 | (1.096) |
| 95% CI | | | | | | | (−4.98, | −2.27) | (−3.82, | 0.51) |
| LSM Diff (SE) | | | | | | | −1.97 | (1.293) | | |
| 95% CI | | | | | | | (−4.52, | 0.58) | | |

| | |
|---|---|
| P-value | 0.1295 |
| (TTM vs. SoC) | |

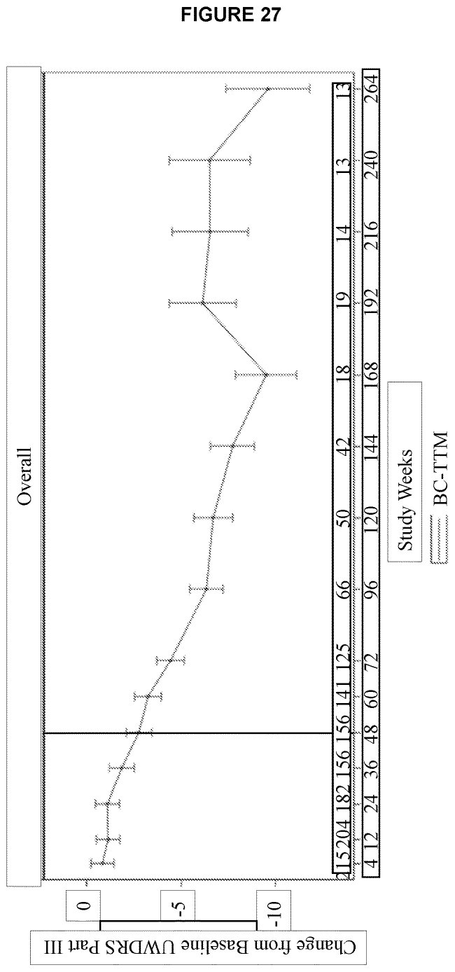

Furthermore, these improvements are seen over 5 years of TTM treatment. These results are reported in Table 31 and FIG. 27.

TABLE 31
UWDRS-III Improvements at 5 Years for Subpopulation E

| TTM Exposure | | Mean | Mean | LSM | LSM |
|---|---|---|---|---|---|
| (Weeks) | N | (SD) | 95% CI | (SE) | 95% CI |
| Baseline | 220 | 15.43 (19.833) | (12.79, 18.06) | NA | NA |
| 4 | 215 | −0.72 (6.701) | (−1.62, 0.18) | −0.83 (0.607) | (−2.03, 0.36) |
| 12 | 204 | −1.11 (7.065) | (−2.08, −0.13)* | −1.13 (0.612) | (−2.34, 0.07) |
| 24 | 182 | −0.97 (8.447) | (−2.21, 0.26) | −1.10 (0.629) | (−2.34, 0.13) |
| 36 | 156 | −2.38 (8.581) | (−3.73, −1.02)* | −1.85 (0.657) | (−3.14, −0.56)* |
| 48 | 156 | −3.37 (9.778) | (−4.91, −1.82)* | −2.76 (0.675) | (−4.09, −1.43)* |
| 60 | 141 | −2.00 (6.40) | (−5.58, −2.18)* | −3.24 (0.700) | (−4.62, −1.87)* |
| 72 | 125 | −4.93 (11.682) | (−7.00, −2.86)* | −4.43 (0.734) | (−5.87, −2.99)* |
| 96 | 66 | −7.95 (14.078) | (−11.41, −4.49)* | −6.32 (0.881) | (−8.05, −4.59)* |
| 120 | 50 | −8.92 (15.045) | (−13.19, −4.64)* | −6.69 (1.027) | (−8.71, −4.68)* |
| 144 | 42 | −9.78 (15.423) | (−14.58, −4.97)* | −7.70 (1.153) | (−9.97, −5.44)* |
| 168 | 18 | −18.08 (19.127) | (−27.59, −8.57)* | −9.48 (1.616) | (−12.66, −6.31)* |
| 192 | 19 | −12.65 (19.476) | (−22.04, −3.27)* | −6.13 (1.783) | (−9.63, −2.63)* |
| 216 | 14 | −16.17 (19.323) | (−27.33, −5.01)* | −6.52 (2.013) | (−10.47, −2.56)* |
| 240 | 13 | −11.48 (16.473) | (−21.43, −1.52)* | −6.49 (2.137) | (−10.68, −2.29)* |
| 264 | 13 | −14.00 (16.513) | (−23.98, −4.02)* | −9.57 (2.222) | (−13.93, −5.21)* |

Note:
Baseline contains observed values and other timepoints are change from baseline.
Note:
LSM and its 95% CI are calculated using an REML based MMRM with fixed effects for visit, cohort, baseline-by-visit interaction, and baseline value as a covariate. The Kenward-Roger approximation is used to estimate degrees of freedom. MMRM was performed for each cohort subset using the same fixed effect terms in the model except for removing cohort term.
Note:
*= statistically significant with two-sided alpha = 0.05 because 95% CI does not contain 0.
Note:
for UWDRS, negative change means improvement.

Moreover, Subpart E patients who received TTM treatment for 48 weeks experienced significant overall clinical improvements, as measured by CGI-I, and significant improvements in disease severity, as measured by CGI-S, relative to SoC treatment. These data are reported in Tables 32 and 33, respectively.

TABLE 32
Transformed Global Improvement (CGI-I) Compared to Baseline for
Subpopulation E (Covariance Structure - UN)

| | Subpopulation E | | |
|---|---|---|---|
| Visit Statistic | | TTM | SoC |

| | | |
|---|---|---|
| No. patients | 88 | 45 |

Week 48, Change from Baseline

| | | |
|---|---|---|
| N | 78 | 39 |

| | | | | |
|---|---|---|---|---|
| Mean (SD) | −0.7 | (0.93) | −0.2 | (0.84) |
| Median (IQR) | 0.0 | (1.0) | 0.0 | (0.0) |
| Q1, Q3 | −1.0, | 0.0 | 0.0, | 0.0 |
| Min, Max | −3, | 1 | −3, | 1 |
| 95% CI | (−0.9, | −0.5) | (−0.4, | 0.1) |
| LSM (SE) | −0.7 | (0.10) | −0.2 | (0.15) |
| 95% CI | (−0.9, | −0.5) | (−0.5, | 0.1) |
| LSM Diff (SE) | −0.5 | (0.18) | | |
| 95% CI | (−0.9, | −0.2) | | |

TABLE 33
CGI-S Compared to Baseline for Subpopulation E

| | Subpopulation E | |
|---|---|---|
| | TTM | SoC |
| Visit Statistic | (N = 104) | (N = 56) |

Baseline Characteristics

| | | | | | |
|---|---|---|---|---|---|
| CGI-S | Mean (SD) | 2.9 | (1.58) | 2.4 | (1.34) |
| | Min, Max | 1, | 6 | 1, | 5 |

Change from Baseline, Week 48

| N | 89 | | 47 | |
|---|---|---|---|---|
| Mean (SD) | −0.4 | (0.79) | −0.1 | (0.73) |
| Median (IQR) | 0.0 | (1.0) | 0.0 | (0.0) |
| Q1, Q3 | −1.0, | 0.0 | 0.0, | 0.0 |
| Min, Max | −3, | 1 | −2, | 2 |
| 95% CI | (−0.6, | −0.2) | (−0.3, | 0.2) |
| LSM (SE) | −0.4 | (0.08) | −0.1 | (0.11) |
| 95% CI | (−0., | −0.2) | (−0.3, | 0.1) |

| | |
|---|---|
| LSM Diff (SE) | −0.2 (0.13) |
| 95% CI | (−0.5, 0.0) |

TTM was generally safe and well-tolerated in the overall safety population of patients with WD and in the Subpart E population who had an incomplete response or were intolerant to SoC therapy at baseline. The majority of the AEs observed in TTM-treated patients were nonserious, mild or moderate in severity, and did not result in treatment withdrawal. Long term data with median exposure of ~approximately 2.5 years (maximum, approximately 6.5 years) and total exposure of 645.6 patient-years in WD patients treated with TTM have shown a safety profile consistent with the data from the primary evaluation period (Study 301).

The risks assessed as associated with TTM treatment were biochemical abnormalities without any significant deleterious clinical outcomes. These abnormalities included liver enzyme elevations, lipid elevations, and neutropenia. These have been generally nonserious, low grade in severity, and with a very limited number of treatment discontinuations. With appropriate monitoring and mitigation strategies, these risks are manageable. The preliminary efficacy and safety data support a positive balance of benefit and risk for TTM treatment in the Subpart E population.

It is understood that the examples and embodiments described herein are for illustrative purposes only and that various modifications or changes in light thereof are suggested to persons skilled in the art and are to be incorporated within the spirit and purview of this application and scope of the appended claims. All publications, patents, and patent applications cited herein are hereby incorporated herein by reference for all purposes.

What is claimed is:

1. Tetrathiomolybdic acid or a pharmaceutically acceptable salt thereof for treating a copper metabolism associated disease or disorder in a subject,
wherein a therapeutically effective amount of tetrathiomolybdic acid or a pharmaceutically acceptable salt thereof is administered to the subject;
wherein the subject is at least 12 years old; and
wherein the subject has an incomplete response and/or is intolerant to a standard of care treatment.

2. The tetrathiomolybdic acid or a pharmaceutically acceptable salt thereof of claim 1,
wherein the copper metabolism-associated disease or disorder is Wilson disease.

3. The tetrathiomolybdic acid or a pharmaceutically acceptable salt thereof of either claim 1 or claim 2, wherein the subject is at least 18 years old.

4. The tetrathiomolybdic acid or a pharmaceutically acceptable salt thereof of any one of claims 1-3, wherein the subject previously received the standard of care treatment for the copper metabolism associated disease or disorder for at least 28 days.

5. The tetrathiomolybdic acid or a pharmaceutically acceptable salt thereof of any one of claims 1-4, wherein the subject previously received a standard of care treatment for the copper metabolism associated disease or disorder for at least 4 weeks, for at least 6 weeks, for at least 12 weeks, for at least 48 weeks, or for at least 72 weeks.

6. The tetrathiomolybdic acid or a pharmaceutically acceptable salt thereof of any one of claims 1-5, wherein the subject previously received a standard of care treatment for the copper metabolism associated disease or disorder for at least 28 months, for at least 36 months, for at least 42 months, or for at least 48 months.

7. The tetrathiomolybdic acid or a pharmaceutically acceptable salt thereof of any one of claims 1-6, wherein the subject previously received a standard of care treatment for the copper metabolism associated disease or disorder for at least 72 months, for at least 96 months, for at least 120 months, or for at least 142 months.

8. The tetrathiomolybdic acid or a pharmaceutically acceptable salt thereof of any one of claims 1-7, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

9. The tetrathiomolybdic acid or a pharmaceutically acceptable salt thereof of any one of claims 1-8, wherein the subject has an incomplete response to the standard of care treatment.

10. The tetrathiomolybdic acid or a pharmaceutically acceptable salt thereof of any one of claims 1-9, wherein the subject has an inadequate response to the standard of care treatment.

11. The tetrathiomolybdic acid or a pharmaceutically acceptable salt thereof of either claim 9 or claim 10, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

12. The tetrathiomolybdic acid or a pharmaceutically acceptable salt thereof of any one of claims 9-11, wherein the subject has a Unified Wilson Disease Rating Scale (UWDRS) Part II total score of greater than 0 or a Unified Wilson Disease Rating Scale (UWDRS) Part III total score of greater than 0 before the therapeutically effective amount of tetrathiomolybdic acid or a pharmaceutically acceptable salt thereof is administered.

13. The tetrathiomolybdic acid or a pharmaceutically acceptable salt thereof of any one of claims 9-11, wherein the subject has a Unified Wilson Disease Rating Scale (UWDRS) Part II total score of greater than 0 and a Unified Wilson Disease Rating Scale (UWDRS) Part III total score of greater than 0 before the therapeutically effective amount of tetrathiomolybdic acid or a pharmaceutically acceptable salt thereof is administered.

14. The tetrathiomolybdic acid or a pharmaceutically acceptable salt thereof of any one of claims 9-13, wherein the subject, before the therapeutically effective amount of tetrathiomolybdic acid or a pharmaceutically acceptable salt thereof is administered, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

15. The tetrathiomolybdic acid or a pharmaceutically acceptable salt thereof of any one of claims 9-14, wherein the subject, before the therapeutically effective amount of tetrathiomolybdic acid or a pharmaceutically acceptable salt thereof is administered, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

16. The tetrathiomolybdic acid or a pharmaceutically acceptable salt thereof of any one of claims 9-15, wherein the subject, before the therapeutically effective amount of tetrathiomolybdic acid or a pharmaceutically acceptable salt thereof is administered, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

17. The tetrathiomolybdic acid or a pharmaceutically acceptable salt thereof of any one of claims 9-16, wherein the subject, before the therapeutically effective amount of tetrathiomolybdic acid or a pharmaceutically acceptable salt thereof is administered, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

18. The tetrathiomolybdic acid or a pharmaceutically acceptable salt thereof of any one of claims 1-8, wherein the subject is intolerant to the standard of care treatment.

19. The tetrathiomolybdic acid or a pharmaceutically acceptable salt thereof of claim 18, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

20. The tetrathiomolybdic acid or a pharmaceutically acceptable salt thereof of either of claim 18 or claim 19, wherein the subject has previously been determined as intolerant to standard of care treatment by a medical professional or has had an adverse event after the receiving the standard of care treatment and before the therapeutically effective amount of tetrathiomolybdic acid or a pharmaceutically acceptable salt thereof is administered.

21. The tetrathiomolybdic acid or a pharmaceutically acceptable salt thereof of any one of claims 18-20, wherein the subject has previously been determined as intolerant to standard of care treatment by a medical professional and has had an adverse event after the receiving the standard of care treatment and before the therapeutically effective amount of tetrathiomolybdic acid or a pharmaceutically acceptable salt thereof is administered.

22. The tetrathiomolybdic acid or a pharmaceutically acceptable salt thereof of any one of claims 1-21, wherein the standard of care treatment comprises trientine, D-penicillamine, and/or zinc.

23. The tetrathiomolybdic acid or a pharmaceutically acceptable salt thereof of any one of claims 1-22, wherein the standard of care treatment comprises trientine and/or D-penicillamine.

24. The tetrathiomolybdic acid or a pharmaceutically acceptable salt thereof of any one of claims 1-23, wherein the therapeutically effective amount of tetrathiomolybdic acid or a pharmaceutically acceptable salt thereof is administered for at least 24 weeks, for at least 36 weeks, or for at least 48 weeks.

25. The tetrathiomolybdic acid or a pharmaceutically acceptable salt thereof of any one of claims 1-24, wherein the therapeutically effective amount of tetrathiomolybdic acid or a pharmaceutically acceptable salt thereof is administered to the subject in a fasted state.

26. The tetrathiomolybdic acid or a pharmaceutically acceptable salt thereof of any one of claims 1-25, wherein the therapeutically effective amount of tetrathiomolybdic acid or a pharmaceutically acceptable salt thereof is in the range of about 7.8 mg to about 60 mg per day.

27. The tetrathiomolybdic acid or a pharmaceutically acceptable salt thereof of any one of claims 1-26, wherein the therapeutically effective amount of tetrathiomolybdic acid or a pharmaceutically acceptable salt thereof is about 7.8 mg daily.

28. The tetrathiomolybdic acid or a pharmaceutically acceptable salt thereof of any one of claims 1-27, wherein the therapeutically effective amount of tetrathiomolybdic acid or a pharmaceutically acceptable salt thereof is about 7.8 mg every other day.

29. The tetrathiomolybdic acid or a pharmaceutically acceptable salt thereof of any one of claims 1-27, wherein the tetrathiomolybdic acid is a pharmaceutically acceptable salt thereof, wherein the pharmaceutically acceptable salt is a bis-choline salt.

30. The tetrathiomolybdic acid or a pharmaceutically acceptable salt thereof of claim 29, wherein the therapeutically effective amount of bis-choline tetrathiomolybdate is administered for at least 24 weeks, for at least 36 weeks, or for at least 48 weeks.

31. The tetrathiomolybdic acid or a pharmaceutically acceptable salt thereof of either claim 29 or claim 30, wherein the therapeutically effective amount of bis-choline tetrathiomolybdate is administered to the subject in a fasted state.

32. The tetrathiomolybdic acid or a pharmaceutically acceptable salt thereof of any one of claims 79-81, wherein the therapeutically effective amount of bis-choline tetrathiomolybdate is in the range of about 15 mg to about 60 mg per day.

33. The tetrathiomolybdic acid or a pharmaceutically acceptable salt thereof of any one of claims 29-32, wherein the therapeutically effective amount of bis-choline tetrathiomolybdate is about 15 mg daily.

34. The tetrathiomolybdic acid or a pharmaceutically acceptable salt thereof of any one of claims 29-33, wherein the therapeutically effective amount of bis-choline tetrathiomolybdate is about 15 mg every other day.

35. The tetrathiomolybdic acid or a pharmaceutically acceptable salt thereof of any one of claims 1-34, wherein the therapeutically effective amount of tetrathiomolybdic acid or a pharmaceutically acceptable salt thereof is sufficient to inhibit progression of neurological damage of the subject.

36. The tetrathiomolybdic acid or a pharmaceutically acceptable salt thereof of any one of claims 1-35, wherein the therapeutically effective amount of tetrathiomolybdic acid or a pharmaceutically acceptable salt thereof is sufficient to inhibit progression of liver damage of the subject.

37. The tetrathiomolybdic acid or a pharmaceutically acceptable salt thereof of any one of claims 1-36, wherein the therapeutically effective amount of tetrathiomolybdic acid or a pharmaceutically acceptable salt thereof is sufficient to improve or maintain one or more of neurological symptoms, psychiatric symptoms, clinical symptoms, disability status, or treatment satisfaction of the subject.

38. The tetrathiomolybdic acid or a pharmaceutically acceptable salt thereof of any one of claims 1-37, wherein the therapeutically effective amount of tetrathiomolybdic acid or a pharmaceutically acceptable salt thereof is sufficient to improve one or more of neurological symptoms, psychiatric symptoms, clinical symptoms, disability status, or treatment satisfaction of the subject.

39. The tetrathiomolybdic acid or a pharmaceutically acceptable salt thereof of claim 38, wherein the improvement is observed after the therapeutically effective amount of tetrathiomolybdic acid or a pharmaceutically acceptable salt thereof is administered for at least 24 weeks, at least 36 weeks, or at least 48 weeks.

40. The tetrathiomolybdic acid or a pharmaceutically acceptable salt thereof of any one of claims 1-39, wherein the therapeutically effective amount of tetrathiomolybdic acid or a pharmaceutically acceptable salt thereof is sufficient to improve a Unified Wilson Disease Rating Scale (UWDRS) Part II total score or a Unified Wilson Disease Rating Scale (UWDRS) Part III total score of the subject.

41. The tetrathiomolybdic acid or a pharmaceutically acceptable salt thereof of any one of claims 1-39, wherein the therapeutically effective amount of tetrathiomolybdic acid or a pharmaceutically acceptable salt thereof is sufficient to improve a Unified Wilson Disease Rating Scale (UWDRS) Part II total score and a Unified Wilson Disease Rating Scale (UWDRS) Part III total score of the subject.

42. The tetrathiomolybdic acid or a pharmaceutically acceptable salt thereof of any one of claims 1-41, wherein the therapeutically effective amount of tetrathiomolybdic acid or a pharmaceutically acceptable salt thereof is sufficient to improve the UWDRS Part II Total Score by at least 5%, at least 10%, at least 15%, or at least 20% of the subject.

43. The tetrathiomolybdic acid or a pharmaceutically acceptable salt thereof of any one of claims 1-42, wherein the therapeutically effective amount of tetrathiomolybdic acid or a pharmaceutically acceptable salt thereof is sufficient to improve the UWDRS Part III Total Score by at least 5%, at least 10%, at least 15%, or at least 20% of the subject.

44. The tetrathiomolybdic acid or a pharmaceutically acceptable salt thereof of any one of claims 40-43, wherein the improvement is observed after the therapeutically effective amount of tetrathiomolybdic acid or a pharmaceutically acceptable salt thereof is administered for least 24 weeks, at least 36 weeks, or at least 48 weeks.

45. The tetrathiomolybdic acid or a pharmaceutically acceptable salt thereof of any one of claims 1-44, wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

46. The tetrathiomolybdic acid or a pharmaceutically acceptable salt thereof of claim 45, wherein the improvement is observed after the therapeutically effective amount of tetrathiomolybdic acid or a pharmaceutically acceptable salt thereof is administered for at least 24 weeks, at least 36 weeks, or at least 48 weeks.

47. The tetrathiomolybdic acid or a pharmaceutically acceptable salt thereof of any one of claims 1-46, wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

48. The tetrathiomolybdic acid or a pharmaceutically acceptable salt thereof of claim 47, wherein the improvement is observed after the therapeutically effective amount of tetrathiomolybdic acid or a pharmaceutically acceptable salt thereof is administered for at least 24 weeks, at least 36 weeks, or at least 48 weeks.

49. The tetrathiomolybdic acid or a pharmaceutically acceptable salt thereof of any one of claims 1-48, wherein the subject is further evaluated for improvements in disability and neurologic symptoms as measured according to Unified Wilson Disease Rating Scale (UWDRS) Part II and/or Part III.

50. The tetrathiomolybdic acid or a pharmaceutically acceptable salt thereof of any one of claims 1-49, wherein the subject is further evaluated for improvements in disability status, psychiatric symptoms, clinical symptoms, treatment satisfaction, or a combination thereof.

51. A method for treating a copper metabolism associated disease or disorder in a subject, the method comprising:
administering to the subject a therapeutically effective amount of tetrathiomolybdic acid or a pharmaceutically acceptable salt thereof;
wherein the subject is at least 12 years old; and
wherein the subject has an incomplete response and/or is intolerant to a standard of care treatment.

52. The method of claim 51, wherein the copper metabolism-associated disease or disorder is Wilson disease.

53. The method of either claim 51 or claim 52, wherein the subject is at least 18 years old.

54. The method of any one of claims 51-53, wherein the subject previously received the standard of care treatment for the copper metabolism associated disease or disorder for at least 28 days.

55. The method of any one of claims 51-54, wherein the subject previously received a standard of care treatment for the copper metabolism associated disease or disorder for at least 4 weeks, for at least 6 weeks, for at least 12 weeks, for at least 48 weeks, or for at least 72 weeks.

56. The method of any one of claims 51-55, wherein the subject previously received a standard of care treatment for the copper metabolism associated disease or disorder for at least 28 months, for at least 36 months, for at least 42 months, or for at least 48 months.

57. The method of any one of claims 51-56, wherein the subject previously received a standard of care treatment for the copper metabolism associated disease or disorder for at least 72 months, for at least 96 months, for at least 120 months, or for at least 142 months.

58. The method of any one of claims 51-57, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

59. The method of any one of claims 51-58, wherein the subject has an incomplete response to the standard of care treatment.

60. The method of claim 51-59, wherein the subject has an inadequate response to the standard of care treatment.

61. The method of either claim 59 or claim 60, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

62. The method of any one of claims 59-61, wherein the subject has a Unified Wilson Disease Rating Scale (UWDRS) Part II total score of greater than 0 or a Unified Wilson Disease Rating Scale (UWDRS) Part III total score of greater than 0 prior to the administering the therapeutically effective amount of tetrathiomolybdic acid or a pharmaceutically acceptable salt thereof.

63. The method of any one of claims 59-61, wherein the subject has a Unified Wilson Disease Rating Scale (UWDRS) Part II total score of greater than 0 and a Unified Wilson Disease Rating Scale (UWDRS) Part III total score of greater than 0 prior to the administering the therapeutically effective amount of tetrathiomolybdic acid or a pharmaceutically acceptable salt thereof.

64. The method of any one of claims 59-63, wherein the subject, prior to the administering the therapeutically effective amount of tetrathiomolybdic acid or a pharmaceutically acceptable salt thereof, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

65. The method of any one of claims 59-64, wherein the subject, prior to the administering the therapeutically effective amount of tetrathiomolybdic acid or a pharmaceutically acceptable salt thereof, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

66. The method of any one of claims 59-55, wherein the subject, prior to the administering the therapeutically effective amount of tetrathiomolybdic acid or a pharmaceutically acceptable salt thereof, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

67. The method of any one of claims 59-56, wherein the subject, prior to the administering the therapeutically effective amount of tetrathiomolybdic acid or a pharmaceutically acceptable salt thereof, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

68. The method of any one of claims 51-58, wherein the subject is intolerant to the standard of care treatment.

69. The method of claim 68, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

70. The method of either of claim 68 or claim 69, wherein the subject has previously been determined as intolerant to standard of care treatment by a medical professional or has had an adverse event after the receiving the standard of care treatment and prior to the administering the therapeutically effective amount of tetrathiomolybdic acid or a pharmaceutically acceptable salt thereof.

71. The method of any one of claims 68-70, wherein the subject has previously been determined as intolerant to standard of care treatment by a medical professional and has had an adverse event after the receiving the standard of care treatment and prior to the administering the therapeutically effective amount of tetrathiomolybdic acid or a pharmaceutically acceptable salt thereof.

72. The method of any one of claims 51-71, wherein the standard of care treatment comprises trientine, D-penicillamine, and/or zinc.

73. The method of any one of claims 51-72, wherein the standard of care treatment comprises trientine and/or D-penicillamine.

74. The method of any one of claims 51-73, wherein the administering of the therapeutically effective amount of tetrathiomolybdic acid or a pharmaceutically acceptable salt thereof is for at least 24 weeks, for at least 36 weeks, or for at least 48 weeks.

75. The method of any one of claims 51-74, wherein the therapeutically effective amount of tetrathiomolybdic acid or a pharmaceutically acceptable salt thereof is administered to the subject in a fasted state.

76. The method of any one of claims 51-75, wherein the therapeutically effective amount of tetrathiomolybdic acid or a pharmaceutically acceptable salt thereof is in the range of about 7.8 mg to about 60 mg per day.

77. The method of any one of claims 51-76, wherein the therapeutically effective amount of tetrathiomolybdic acid or a pharmaceutically acceptable salt thereof is about 7.8 mg daily.

78. The method of any one of claims 51-77, wherein the therapeutically effective amount of tetrathiomolybdic acid or a pharmaceutically acceptable salt thereof is about 7.8 mg every other day.

79. The method of any one of claims 51-77, wherein the tetrathiomolybdic acid is a pharmaceutically acceptable salt thereof, wherein the pharmaceutically acceptable salt is a bis-choline salt.

80. The method of claim 79, wherein the administering of the therapeutically effective amount of bis-choline tetrathiomolybdate is for at least 24 weeks, for at least 36 weeks, or for at least 48 weeks.

81. The method of either claim 79 or claim 80, wherein the therapeutically effective amount of bis-choline tetrathiomolybdate is administered to the subject in a fasted state.

82. The method of any one of claims 79-81, wherein the therapeutically effective amount of bis-choline tetrathiomolybdate is in the range of about 15 mg to about 60 mg per day.

83. The method of any one of claims 79-82, wherein the therapeutically effective amount of bis-choline tetrathiomolybdate is about 15 mg daily.

84. The method of any one of claims 79-83, wherein the therapeutically effective amount of bis-choline tetrathiomolybdate is about 15 mg every other day.

85. The method of any one of claims 51-84, wherein the therapeutically effective amount of tetrathiomolybdic acid or a pharmaceutically acceptable salt thereof is sufficient to inhibit progression of neurological damage of the subject.

86. The method of any one of claim 51-85, wherein the therapeutically effective amount of tetrathiomolybdic acid or a pharmaceutically acceptable salt thereof is sufficient to inhibit progression of liver damage of the subject.

87. The method of any one of claims 51-86, wherein the therapeutically effective amount of tetrathiomolybdic acid or a pharmaceutically acceptable salt thereof is sufficient to improve or maintain one or more of neurological symptoms, psychiatric symptoms, clinical symptoms, disability status, or treatment satisfaction of the subject.

88. The method of any one of claims 51-87, wherein the therapeutically effective amount of tetrathiomolybdic acid or a pharmaceutically acceptable salt thereof is sufficient to improve one or more of neurological symptoms, psychiatric symptoms, clinical symptoms, disability status, or treatment satisfaction of the subject.

89. The method of claim 88, wherein the improvement is observed after at least 24 weeks, at least 36 weeks, or at least 48 weeks of administering the therapeutically effective amount of tetrathiomolybdic acid or a pharmaceutically acceptable salt thereof.

90. The method of any one of claims 51-89, wherein the therapeutically effective amount of tetrathiomolybdic acid or a pharmaceutically acceptable salt thereof is sufficient to improve a Unified Wilson Disease Rating Scale (UWDRS) Part II total score or a Unified Wilson Disease Rating Scale (UWDRS) Part III total score of the subject.

91. The method of any one of claims 51-89, wherein the therapeutically effective amount of tetrathiomolybdic acid or a pharmaceutically acceptable salt thereof is sufficient to improve a Unified Wilson Disease Rating Scale (UWDRS) Part II total score and a Unified Wilson Disease Rating Scale (UWDRS) Part III total score of the subject.

92. The method of any one of claims 51-91, wherein the therapeutically effective amount of tetrathiomolybdic acid or a pharmaceutically acceptable salt thereof is sufficient to improve the UWDRS Part II Total Score by at least 5%, at least 10%, at least 15%, or at least 20% of the subject.

93. The method of any one of claims 51-92, wherein the therapeutically effective amount of tetrathiomolybdic acid or a pharmaceutically acceptable salt thereof is sufficient to improve the UWDRS Part III Total Score by at least 5%, at least 10%, at least 15%, or at least 20% of the subject.

94. The method of any one of claims 90-93, wherein the improvement is observed after at least 24 weeks, at least 36 weeks, or at least 48 weeks of administering the therapeutically effective amount of tetrathiomolybdic acid or a pharmaceutically acceptable salt thereof.

95. The method of any one of claims 51-4494 wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

96. The method of claim 95, wherein the improvement is observed after at least 24 weeks, at least 36 weeks, or at least 48 weeks of administering the therapeutically effective amount of tetrathiomolybdic acid or a pharmaceutically acceptable salt thereof.

97. The method of any one of claims 51-96, wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

98. The method of claim 97, wherein the improvement is observed after at least 24 weeks, at least 36 weeks, or at least 48 weeks of administering the therapeutically effective amount of tetrathiomolybdic acid or a pharmaceutically acceptable salt thereof.

99. The method of any one of claims 51-98, further comprising evaluating the subjects for improvements in disability and neurologic symptoms as measured according to Unified Wilson Disease Rating Scale (UWDRS) Part II and/or Part III.

100. The method of any one of claims 51-99, further comprising evaluating the subjects for improvements in disability status, psychiatric symptoms, clinical symptoms, treatment satisfaction, or a combination thereof.

101. A use of tetrathiomolybdic acid or a pharmaceutically acceptable salt thereof for the manufacture of a medicament for treating a copper metabolism associated disease or disorder in a subject,
wherein a therapeutically effective amount of tetrathiomolybdic acid or a pharmaceutically acceptable salt thereof is administered to the subject;
wherein the subject is at least 12 years old; and
wherein the subject has an incomplete response and/or is intolerant to a standard of care treatment.

102. The use of claim 101, wherein the copper metabolism-associated disease or disorder is Wilson disease.

103. The use of either claim 101 or claim 102, wherein the subject is at least 18 years old.

104. The use of any one of claims 101-103, wherein the subject previously received the standard of care treatment for the copper metabolism associated disease or disorder for at least 28 days.

105. The use of any one of claims 101-104, wherein the subject previously received a standard of care treatment for the copper metabolism associated disease or disorder for at least 4 weeks, for at least 6 weeks, for at least 12 weeks, for at least 48 weeks, or for at least 72 weeks.

106. The use of any one of claims 101-105, wherein the subject previously received a standard of care treatment for the copper metabolism associated disease or disorder for at least 28 months, for at least 36 months, for at least 42 months, or for at least 48 months.

107. The use of any one of claims 101-106, wherein the subject previously received a standard of care treatment for the copper metabolism associated disease or disorder for at least 72 months, for at least 96 months, for at least 120 months, or for at least 142 months.

108. The use of any one of claims 101-107, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

109. The use of any one of claims 101-108, wherein the subject has an incomplete response to the standard of care treatment.

110. The use of claim 101-109, wherein the subject has an inadequate response to the standard of care treatment.

111. The use of either claim 109 or claim 110, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

112. The use of any one of claims 109-111, wherein the subject has a Unified Wilson Disease Rating Scale (UWDRS) Part II total score of greater than 0 or a Unified Wilson Disease Rating Scale (UWDRS) Part III total score of greater than 0 before the therapeutically effective amount of tetrathiomolybdic acid or a pharmaceutically acceptable salt thereof is administered.

113. The use of any one of claims 109-111, wherein the subject has a Unified Wilson Disease Rating Scale (UWDRS) Part II total score of greater than 0 and a Unified Wilson Disease Rating Scale (UWDRS) Part III total score of greater than 0 before the therapeutically effective amount of tetrathiomolybdic acid or a pharmaceutically acceptable salt thereof is administered.

114. The use of any one of claims 109-113, wherein the subject, before the therapeutically effective amount of tetrathiomolybdic acid or a pharmaceutically acceptable salt thereof is administered, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

115. The use of any one of claims 109-114, wherein the subject, before the therapeutically effective amount of tetrathiomolybdic acid or a pharmaceutically acceptable salt thereof is administered, has a Unified Wilson Disease Rating Scale (UWDRS) Part II of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

116. The use of any one of claims 109-115, wherein the subject, before the therapeutically effective amount of tetrathiomolybdic acid or a pharmaceutically acceptable salt thereof is administered, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

117. The use of any one of claims 109-116, wherein the subject, before the therapeutically effective amount of tetrathiomolybdic acid or a pharmaceutically acceptable salt thereof is administered, has a Unified Wilson Disease Rating Scale (UWDRS) Part III of greater than 0, greater than 1, greater than 2, or greater than 3 for at least one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

118. The use of any one of claims 101-108, wherein the subject is intolerant to the standard of care treatment.

119. The use of claim 118, wherein the subject has symptoms associated with the copper metabolism associated disease or disorder after receiving the standard of care treatment for at least 26 weeks, for at least 52 weeks, for at least 72 weeks, for at least 84 weeks, or for at least 96 weeks.

120. The use of either of claim 118 or claim 119, wherein the subject has previously been determined as intolerant to standard of care treatment by a medical professional or has had an adverse event after the receiving the standard of care treatment and before the therapeutically effective amount of tetrathiomolybdic acid or a pharmaceutically acceptable salt thereof is administered.

121. The use of any one of claims 118-120, wherein the subject has previously been determined as intolerant to standard of care treatment by a medical professional and has had an adverse event after the receiving the standard of care treatment and before the therapeutically effective amount of tetrathiomolybdic acid or a pharmaceutically acceptable salt thereof is administered.

122. The use of any one of claims 101-121, wherein the standard of care treatment comprises trientine, D-penicillamine, and/or zinc.

123. The use of any one of claims 101-122, wherein the standard of care treatment comprises trientine and/or D-penicillamine.

124. The use of any one of claims 101-123, wherein the therapeutically effective amount of tetrathiomolybdic acid or a pharmaceutically acceptable salt thereof is administered for at least 24 weeks, for at least 36 weeks, or for at least 48 weeks.

125. The use of any one of claims 101-124, wherein the therapeutically effective amount of tetrathiomolybdic acid or a pharmaceutically acceptable salt thereof is administered to the subject in a fasted state.

126. The use of any one of claims 101-125, wherein the therapeutically effective amount of tetrathiomolybdic acid or a pharmaceutically acceptable salt thereof is in the range of about 7.8 mg to about 60 mg per day.

127. The use of any one of claims 101-126, wherein the therapeutically effective amount of tetrathiomolybdic acid or a pharmaceutically acceptable salt thereof is in the range of about 7.8 mg daily.

128. The use of any one of claims 101-127, wherein the therapeutically effective amount of tetrathiomolybdic acid or a pharmaceutically acceptable salt thereof is in the range of about 7.8 mg every other day.

129. The use of any one of claims 101-127, wherein the tetrathiomolybdic acid is a pharmaceutically acceptable salt thereof, wherein the pharmaceutically acceptable salt is a bis-choline salt.

130. The use of claim 129, wherein the therapeutically effective amount of bis-choline tetrathiomolybdate is administered for at least 24 weeks, for at least 36 weeks, or for at least 48 weeks.

131. The use of either claim 129 or claim 130, wherein the therapeutically effective amount of bis-choline tetrathiomolybdate is administered to the subject in a fasted state.

132. The use of any one of claims 129-131, wherein the therapeutically effective amount of bis-choline tetrathiomolybdate is in the range of about 15 mg to about 60 mg per day.

133. The use of any one of claims 129-132, wherein the therapeutically effective amount of bis-choline tetrathiomolybdate is about 15 mg daily.

134. The use of any one of claims 129-133, wherein the therapeutically effective amount of bis-choline tetrathiomolybdate is about 15 mg every other day.

135. The use of any one of claims 101-134, wherein the therapeutically effective amount of tetrathiomolybdic acid or a pharmaceutically acceptable salt thereof is sufficient to inhibit progression of neurological damage of the subject.

136. The use of any one of claims 101-135, wherein the therapeutically effective amount of tetrathiomolybdic acid or a pharmaceutically acceptable salt thereof is sufficient to inhibit progression of liver damage of the subject.

137. The use of any one of claims 101-136, wherein the therapeutically effective amount of tetrathiomolybdic acid or a pharmaceutically acceptable salt thereof is sufficient to improve or maintain one or more of neurological symptoms, psychiatric symptoms, clinical symptoms, disability status, or treatment satisfaction of the subject.

138. The use of any one of claims 101-137, wherein the therapeutically effective amount of tetrathiomolybdic acid or a pharmaceutically acceptable salt thereof is sufficient to improve one or more of neurological symptoms, psychiatric symptoms, clinical symptoms, disability status, or treatment satisfaction of the subject.

139. The use of claim 138, wherein the improvement is observed after the therapeutically effective amount of tetrathiomolybdic acid or a pharmaceutically acceptable salt thereof is administered for at least 24 weeks, at least 36 weeks, or at least 48 weeks.

140. The use of any one of claims 101-139, wherein the therapeutically effective amount of tetrathiomolybdic acid or a pharmaceutically acceptable salt thereof is sufficient to improve a Unified Wilson Disease Rating Scale (UWDRS) Part II total score or a Unified Wilson Disease Rating Scale (UWDRS) Part III total score of the subject.

141. The use of any one of claims 101-139, wherein the therapeutically effective amount of tetrathiomolybdic acid or a pharmaceutically acceptable salt thereof is sufficient to improve a Unified Wilson Disease Rating Scale (UWDRS) Part II total score and a Unified Wilson Disease Rating Scale (UWDRS) Part III total score of the subject.

142. The use of any one of claims 101-141, wherein the therapeutically effective amount of tetrathiomolybdic acid or a pharmaceutically acceptable salt thereof is sufficient to improve the UWDRS Part II Total Score by at least 5%, at least 10%, at least 15%, or at least 20% of the subject.

143. The use of any one of claims 101-142, wherein the therapeutically effective amount of tetrathiomolybdic acid or a pharmaceutically acceptable salt thereof is sufficient to improve the UWDRS Part III Total Score by at least 5%, at least 10%, at least 15%, or at least 20% of the subject.

144. The use of any one of claims 140-143, wherein the improvement is observed after the therapeutically effective amount of tetrathiomolybdic acid or a pharmaceutically acceptable salt thereof is administered for at least 24 weeks, at least 36 weeks, or at least 48 weeks.

145. The use of any one of claims 101-144, wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, facial expression, finger to nose, finger taps, gait, handwriting, head tremor, involuntary crying, jaw tremor, leg agility, oculomotor, postural arm tremor, postural leg tremor, posture, pyramidal signs, RAM of hands, rigidity, speech, stereotyped actions, tremor at rest, and wing-beating tremor.

146. The use of claim 145, wherein the improvement is observed after the therapeutically effective amount of tetrathiomolybdic acid or a pharmaceutically acceptable salt thereof is administered for at least 24 weeks, at least 36 weeks, or at least 48 weeks.

147. The use of any one of claims 101-146, wherein the therapeutically effective amount of tetrathiomolybdic acid or a pharmaceutically acceptable salt thereof is sufficient to improve one or more neurological symptoms of the subject as measured according to Unified Wilson Disease Rating Scale (UWDRS) Part II and/or Part III, the one or more neurological symptoms selected from the group consisting of arising from a chair, arm and hand dystonia, cervical dystonia, chorea, finger to nose, finger taps, handwriting, head tremor, jaw tremor, leg agility, RAM of hands, rigidity, speech, and tremor at rest.

148. The use of claim 147, wherein the improvement is observed after the therapeutically effective amount of tetrathiomolybdic acid or a pharmaceutically acceptable salt thereof is administered for at least 24 weeks, at least 36 weeks, or at least 48 weeks.

149. The use of any one of claims 101-148, wherein the subject is further evaluated for improvements in disability and neurologic symptoms as measured according to Unified Wilson Disease Rating Scale (UWDRS) Part II and/or Part III.

150. The use of any one of claims 101-149, wherein the subject is further evaluated for improvements in disability status, psychiatric symptoms, clinical symptoms, treatment satisfaction, or a combination thereof.
